SEQUENCE LISTING (11 sequences)

SEQ ID NO: 1 - protein, 1935 aa, Homo sapiens
MGDSEMAVFGAAAPYLRKSEKERLEAQTRPFDLKKDVFVPDDKQEFVKAKIVSREGGKVTAETEYGKTVTVKEDQVMQQNPPKFDKIEDMAMLTFLHEPAVLYNLKDRYGSWMIYTYSGLFCVTVNPYKWLPVYTPEVVAAYRGKKRSEAPPHIFSISDNAYQYMLTDRENQSILITGESGAGKTVNTKRVIQYFAVIAAIGDRSKKDQSPGKGTLEDQIIQANPALEAFGNAKTVRNDNSSRFGKFIRIHFGATGKLASADIETYLLEKSRVIFQLKAERDYHIFYQILSNKKPELLDMLLITNNPYDYAFISQGETTVASIDDAEELMATDNAFDVLGFTSEEKNSMYKLTGAIMHFGNMKFKLKQREEQAEPDGTEEADKSAYLMGLNSADLLKGLCHPRVKVGNEYVTKGQNVQQVIYATGALAKAVYERMFNWMVTRINATLETKQPRQYFIGVLDIAGFEIFDFNSFEQLCINFTNEKLQQFFNHHMFVLEQEEYKKEGIEWTFIDFGMDLQACIDLIEKPMGIMSILEEECMFPKATDMTFKAKLFDNHLGKSANFQKPRNIKGKPEAHFSLIHYAGIVDYNIIGWLQKNKDPLNETVVGLYQKSSLKLLSTLFANYAGADAPIEKGKGKAKKGSSFQTVSALHRENLNKLMTNLRSTHPHFVRCIIPNETKSPGVMDNPLVMHQLRCNGVLEGIRICRKGFPNRILYGDFRQRYRILNPAAIPEGQFIDSRKGAEKLLSSLDIDHNQYKFGHTKVFFKAGLLGLLEEMRDERLSRIITRIQAQSRGVLARMEYKKLLERRDSLLVIQWNIRAFMGVKNWPWMKLYFKIKPLLKSAEREKEMASMKEEFTRLKEALEKSEARRKELEEKMVSLLQEKNDLQLQVQAEQDNLADAEERCDQLIKNKIQLEAKVKEMNERLEDEEEMNAELTAKKRKLEDECSELKRDIDDLELTLAKVEKEKHATENKVKNLTEEMAGLDEIIAKLTKEKKALQEAHQQALDDLQAEEDKVNTLTKAKVKLEQQVDDLEGSLEQEKKVRMDLERAKRKLEGDLKLTQESIMDLENDKQQLDERLKKKDFELNALNARIEDEQALGSQLQKKLKELQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKMCRTLEDQMNEHRSKAEETQRSVNDLTSQRAKLQTENGELSRQLDEKEALISQLTRGKLTYTQQLEDLKRQLEEEVKAKNALAHALQSARHDCDLLREQYEEETEAKAELQRVLSKANSEVAQWRTKYETDAIQRTEELEEAKKKLAQRLQEAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKRENKNLQEEISDLTEQLGSSGKTIHELEKVRKQLEAEKMELQSALEEAEASLEHEEGKILRAQLEFNQIKAEIERKLAEKDEEMEQAKRNHLRVVDSLQTSLDAETRSRNEALRVKKKMEGDLNEMEIQLSHANRMAAEAQKQVKSLQSLLKDTQIQLDDAVRANDDLKENIAIVERRNNLLQAELEELRAVVEQTERSRKLAEQELIETSERVQLLHSQNTSLINQKKKMDADLSQLQTEVEEAVQECRNAEEKAKKAITDAAMMAEELKKEQDTSAHLERMKKNMEQTIKDLQHRLDEAEQIALKGGKKQLQKLEARVRELENELEAEQKRNAESVKGMRKSERRIKELTYQTEEDRKNLLRLQDLVDKLQLKVKAYKRQAEEAEEQANTNLSKFRKVQHELDEAEERADIAESQVNKLRAKSRDIGTKGLNEE

SEQ ID NO: 2 - protein, 736 aa, Adeno-associated virus serotype 9
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKSGQQPAKKRLNFGQTGDSESVPDPQPLGEPPEAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDEDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 3 - protein, 736 aa, Adeno-associated virus serotype 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 4 - DNA, 2208 bp, Adeno-associated virus
atggctgccgatggttatcttccagattggctcgaggacaacctctctgagggcattcgcgagtggtgggacctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcagtcaccccaagaaccagactcatcctcgggcatcggcaaatcaggccagcagcccgctaaaaagagactcaattttggtcagactggcgactcagagtcagtccccgacccacaacctctcggagaacctccagaagccccctcaggtctgggacctaatacaatggcttcaggcggtggcgctccaatggcagacaataacgaaggcgccgacggagtgggtaattcctcgggaaattggcattgcgattccacatggctgggggacagagtcatcaccaccagcacccgaacctgggcattgcccacctacaacaaccacctctacaagcaaatctccaatggaacatcgggaggaagcaccaacgacaacacctactttggctacagcaccccctgggggtattttgacttcaacagattccactgccacttctcaccacgtgactggcagcgactcatcaacaacaactggggattccggccaaagagactcaacttcaagctgttcaacatccaggtcaaggaggttacgacgaacgaaggcaccaagaccatcgccaataaccttaccagcaccgtccaggtctttacggactcggagtaccagctaccgtacgtcctaggctctgcccaccaaggatgcctgccaccgtttcctgcagacgtcttcatggttcctcagtacggctacctgacgctcaacaatggaagtcaagcgttaggacgttcttctttctactgtctggaatacttcccttctcagatgctgagaaccggcaacaactttcagttcagctacactttcgaggacgtgcctttccacagcagctacgcacacagccagagtctagatcgactgatgaaccccctcatcgaccagtacctatactacctggtcagaacacagacaactggaactgggggaactcaaactttggcattcagccaagcaggccctagctcaatggccaatcaggctagaaactgggtacccgggccttgctaccgtcagcagcgcgtctccacaaccaccaaccaaaataacaacagcaactttgcgtggacgggagctgctaaattcaagctgaacgggagagactcgctaatgaatcctggcgtggctatggcatcgcacaaagacgacgaggaccgcttctttccatcaagtggcgttctcatatttggcaagcaaggagccgggaacgatggagtcgactacagccaggtgctgattacagatgaggaagaaattaaagccaccaaccctgtagccacagaggaatacggagcagtggccatcaacaaccaggccgctaacacgcaggcgcaaactggacttgtgcataaccagggagttattcctggtatggtctggcagaaccgggacgtgtacctgcagggccctatttgggctaaaatacctcacacagatggcaactttcacccgtctcctctgatgggtggatttggactgaaacacccacctccacagattctaattaaaaatacaccagtgccggcagatcctcctcttaccttcaatcaagccaagctgaactctttcatcacgcagtacagcacgggacaagtcagcgtggaaatcgagtgggagctgcagaaagaaaacagcaagcgctggaatccagagatccagtatacttcaaactactacaaatctacaaatgtggactttgctgtcaataccgaaggtgtttactctgagcctcgccccattggtactcgttacctcacccgtaatttg

SEQ ID NO: 5 - DNA, 2211 bp, Adeno-associated virus
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 6 - DNA, 8159 bp, artificial
  AAV vector
ggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcggtaccggtcgccaccatggactacaaagaccatgacggtgattataaagatcatgacatcgattacaaggatgacgatgacaagcttggtaccgagctcggatccatgggagattctgaaatggccgtcttcggcgctgccgcaccatatctccgcaaaagtgaaaaggagcgcctggaggcccaaaccagacccttcgatctgaaaaaggatgtgtttgtcccggatgacaagcaagaatttgtaaaagcaaagatcgtctctcgggagggcggtaaagtgactgctgaaacagaatacggcaaaaccgtcactgtcaaagaagatcaggtaatgcaacaaaatcctccgaaatttgataaaatcgaagacatggccatgttgacgtttttgcatgagccagctgttttgtataatctgaaggatcggtacggatcctggatgatttacacatactcaggcttgttctgcgtgaccgttaatccctataagtggctgcctgtctacacgccggaggttgttgcagcatatagggggaaaaagaggtccgaagcccctccccacatcttctccatttctgacaacgcctaccagtatatgctgactgacagggagaaccaaagcattcttattaccggagagtctggggcgggcaagacggtaaatacaaaacgcgtgatacagtactttgcagttattgctgcgattggcgacaggagtaaaaaggatcagagccccggcaagggtactctcgaagatcaaattatccaggctaatcccgccctcgaagcgttcgggaatgccaaaacggtccgcaatgataacagtagcagatttgggaagtttatccgcatccatttcggagcgactggcaaactcgcctcagcagacatcgagacatacctgttggaaaagtcacgagtgatatttcagctcaaagccgagcgggactatcatatcttttaccagatactttctaacaaaaaaccagagcttcttgacatgcttctgatcactaacaatccgtatgactatgcattcatcagtcagggagagaccacggttgcgtcaatagacgatgctgaggagctgatggccacagacaatgccttcgatgtgctcggatttactagcgaggagaagaacagcatgtacaagcttaccggtgctatcatgcacttcggcaatatgaaatttaaactcaagcagcgggaagaacaggcggagccggacggaactgaagaggctgataaaagtgcctacctcatggggcttaactctgccgacttgcttaaaggattgtgtcatccacgggtaaaggtaggtaatgaatatgtaactaagggtcaaaatgtgcaacaagtcatatacgcgactggggctctggcaaaagccgtttacgagagaatgtttaactggatggtcacacgaattaatgccactctggagacaaaacaaccccggcagtactttataggtgtgctggatatcgcaggtttcgaaattttcgatttcaactctttcgagcaactttgtatcaatttcaccaacgagaagctccaacaattttttaaccatcatatgttcgttctcgagcaagaggaatacaaaaaagagggcatcgaatggactttcatcgatttcggtatggatcttcaggcttgtatagatctcatcgagaaaccgatggggattatgagtattctggaggaggaatgtatgttcccgaaagccactgacatgacatttaaggccaagctgttcgataatcacttggggaagtccgccaatttccagaaacctaggaacataaaaggtaagccggaggcgcacttctctcttatccattacgcgggaatcgtcgattataacattattggctggcttcaaaagaacaaggatccgcttaacgaaacagtggtcggcctttatcagaaaagctcactgaaacttcttagtacgctctttgctaattacgctggtgctgacgctccaatcgagaaaggcaagggaaaagctaagaaaggcagtagctttcaaacagtctctgccctgcacagagagaatctcaacaagctcatgaccaatctgcggagcacacatccacattttgtcaggtgcataatccctaatgagacgaaaagtccaggcgtaatggacaatccgctggttatgcaccagctcagatgtaatggagtattggaaggtatacgcatatgtagaaaaggcttcccaaacaggatactgtatggtgacttccgacaacgctatcgaatactgaaccccgcagccattcccgagggacaattcatagacagccgcaagggagcggaaaagcttctttcctccctcgacatagaccataaccagtacaaatttgggcacaccaaagtatttttcaaagcgggccttcttggactgttggaagagatgcgcgatgaaaggctgtcaaggataataaccaggattcaagcgcaatctcggggagtgctcgcgaggatggagtacaagaaattgttggaacggagagattccttgcttgtgattcagtggaatatacgagctttcatgggagtaaaaaactggccttggatgaaactctactttaagataaagccactgctgaagtccgcagagagggagaaggagatggcatcaatgaaggaggaatttacgcgcttgaaagaggctctcgagaagtccgaagctagacggaaggaattggaagagaagatggtatctcttttgcaggagaagaatgatcttcagttgcaagtacaagcggaacaagacaatctcgccgacgcagaggaaaggtgtgatcagttgatcaaaaataaaatccaactggaagccaaggtgaaagaaatgaatgagcgcttggaggacgaagaagagatgaatgcagagttgactgcgaagaagcggaagctcgaagacgaatgcagcgagctcaagcgcgacattgacgacctggaattgacccttgccaaagtggaaaaggagaaacatgcaaccgagaataaggtaaagaatctgacagaagagatggcgggacttgacgagattatcgcgaagcttacaaaggagaagaaggcccttcaggaagcgcaccaacaggcccttgacgatcttcaggccgaggaagacaaagtgaacacattgactaaagctaaagtgaagcttgagcaacaggtcgatgacgaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagatgtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagagctcggatcctgagaacttcagggtgagtctatgggac

SEQ ID NO: 7 - DNA, 8529 bp, artificial
  AAV vector
ggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagcgtaatggacaatccgctggttatgcaccagctcagatgtaatggagtattggaaggtatacgcatatgtagaaaaggcttcccaaacaggatactgtatggtgacttccgacaacgctatcgaatactgaaccccgcagccattcccgagggacaattcatagacagccgcaagggagcggaaaagcttctttcctccctcgacatagaccataaccagtacaaatttgggcacaccaaagtatttttcaaagcgggccttcttggactgttggaagagatgcgcgatgaaaggctgtcaaggataataaccaggattcaagcgcaatctcggggagtgctcgcgaggatggagtacaagaaattgttggaacggagagattccttgcttgtgattcagtggaatatacgagctttcatgggagtaaaaaactggccttggatgaaactctactttaagataaagccactgctgaagtccgcagagagggagaaggagatggcatcaatgaaggaggaatttacgcgcttgaaagaggctctcgagaagtccgaagctagacggaaggaattggaagagaagatggtatctcttttgcaggagaagaatgatcttcagttgcaagtacaagcggaacaagacaatctcgccgacgcagaggaaaggtgtgatcagttgatcaaaaataaaatccaactggaagccaaggtgaaagaaatgaatgagcgcttggaggacgaagaagagatgaatgcagagttgactgcgaagaagcggaagctcgaagacgaatgcagcgagctcaagcgcgacattgacgacctggaattgacccttgccaaagtggaaaaggagaaacatgcaaccgagaataaggtaaagaatctgacagaagagatggcgggacttgacgagattatcgcgaagcttacaaaggagaagaaggcccttcaggaagcgcaccaacaggcccttgacgatcttcaggccgaggaagacaaagtgaacacattgactaaagctaaagtgaagcttgagcaacaggtcgatgacctggaaggctcacttgagcaagagaagaaagtgcgcatggatctcgagcgcgctaaacgcaagctcgagggagatttgaagctgactcaagagtcaattatggaccttgaaaacgacaagcaacagttggatgagagacttaagaagaaggacttcgaactcaacgcacttaatgccaggatcgaagatgaacaagctctcgggtctcagctccaaaagaagcttaaggaactccaggcacgcatagaagaactcgaagaagagttggaagccgagagaacggcgagagctaaagtggaaaaactccgaagcgatttgtccagagagctggaagagatttccgagcggcttgaggaggcgggaggcgcgacgagcgtgcagatcgaaatgaataagaagagagaagctgaatttcaaaaaatgcgccgagaccttgaagaagcaacactccagcacgaagccacagcagctgcgcttcggaagaagcatgctgattctgtcgcagagctgggggagcagatcgacaatctccagcgagtcaaacaaaaactggaaaaggagaaatctgagtttaaattggagcttgacgacgtcacgtctaacatggagcagataataaaggcaaaagcaaaccttgagaaaatgtgccgcactcttgaggaccagatgaacgagcatcgcagtaaggcggaagagactcagagatctgttaacgacttgaccagtcaacgagctaaacttcagacagaaaatggagaactctcccgacaactggacgaaaaggaggccctgatcagccagttgactaggggtaaactcacctacacacagcagcttgaggacttgaagcgccaactcgaagaggaggtgaaagctaaaaacgcactggcacatgcgctgcaatccgcaagacatgactgcgacctcctgcgggaacagtacgaagaagaaactgaggcgaaagctgagctccaacgggtactttctaaggcaaattctgaagttgcacagtggcgaactaagtacgaaaccgatgcgatccaacgcacggaggagttggaggaagctaaaaagaagctcgcacagcgactgcaggaagccgaagaagccgttgaggcagttaatgcaaaatgcagtagtctcgagaagactaaacatagactccagaacgaaatagaggatctgatggtggatgttgaacgctccaatgctgctgcggcagccctcgacaaaaaacagcggaatttcgataagattcttgcggaatggaagcagaaatacgaagagtctcagagtgaactggagagctcccaaaaggaggcccgaagtctgtctaccgaattgtttaaactcaagaacgcgtacgaagaatctctggaacacttggaaacctttaaaagagagaacaaaaacctgcaagaggaaataagtgaccttaccgagcaactggggagctccgggaaaaccatccacgaattggaaaaagtcaggaaacagttggaggcagagaaaatggagctccaatccgccctggaggaagcggaagcatctctggaacacgaggagggcaagattctgagggcccaactggaatttaaccagatcaaggcagagatagaacgaaaactcgcggagaaggacgaagagatggagcaggcgaaacggaatcatctgcgggttgtagactccctgcaaacctccctcgatgccgaaaccagaagccggaatgaggcccttcgggtgaaaaaaaaaatggagggcgacttgaacgaaatggaaattcaactttctcacgccaaccgcatggcggccgaggcgcagaaacaggtaaaatctctccagtctctcctcaaagatacacaaatccaactcgacgatgctgtaagggcaaacgatgatttgaaagagaatatagcaatcgtcgagcgccgcaataatctgttgcaagcagagcttgaagaactgcgcgcggtcgtagaacagaccgaacgcagtagaaagttggctgagcaggaacttattgagacttccgagcgcgttcagcttctgcattcccagaacacctctctgattaatcagaagaaaaagatggacgcagatctgtctcagttgcagacggaagtggaagaagctgttcaagagtgccggaacgcggaggaaaaagctaaaaaggcgataactgatgcagcgatgatggctgaggaactgaagaaggagcaagacacctccgcccatttggagcgaatgaagaagaatatggaacaaactattaaggatctccagcaccggcttgatgaggctgaacagatcgccttgaaaggggggaaaaagcaattgcagaaattggaagcccgagtaagggaactggagaatgagttggaagctgagcaaaagcggaacgctgagtctgtgaagggaatgagaaagagtgaacggaggataaaagaactcacatatcaaacggaagaggaccggaaaaacttgctccgcctccaagacctcgttgacaagcttcaattgaaagtcaaggcttataaaagacaagctgaagaagctgaagaacaagcgaacaccaatttgtccaaatttcggaaagtacagcatgagctcgacgaggctgaggagcgggctgacatagctgagtcccaggtcaataaactgcgagcgaaaagcagagacattggcaccaaaggtttgaatgaagagaccggtgagggcagaggaagtcttctaacatgcggtgacgtggaggagaatcccggcccttccggtatgaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaataagc

SEQ ID NO: 8 - DNA, 8821 bp, artificial
  AAV vector
ggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcggtaccggtcgccaccatgatggccaaactcacttctgcagtcccagtcctcacagcaagggatgttgcaggggctgtagagttctggactgacagattaggattctccagagactttgttgaagatgattttgctggtgttgtcagagatgatgtcaccctcttcatctcagcagttcaggaccaagttgtccctgacaacacccttgcttgggtctgggtcagaggcctagatgagctttatgcagaatggtcagaagtagtcagcacaaatttcagggatgcctctggcccagccatgacagaaattggtgaacaaccttggggaagggaatttgccctcagagaccctgctggaaattgtgtccattttgtagctgaggaacaggacggatctggagcaacaaacttctcactactcaaacaagcaggtgacgtggaggagaatcccgggcctgactacaaagaccatgacggtgattataaagatcatgacatcgattacaaggatgacgatgacaagcttgagctcggatccatgggagattctgaaatggccgtcttcggcgctgccgcaccatatctccgcaaaagtgaaaaggagcgcctggaggcccaaaccagacccttcgatctgaaaaaggatgtgtttgtcccggatgacaagcaagaatttgtaaaagcaaagatcgtctctcgggagggcggtaaagtgactgctgaaacagaatacggcaaaaccgtcactgtcaaagaagatcaggtaatgcaacaaaatcctccgaaatttgataaaatcgaagacatggccatgttgacgtttttgcatgagccagctgttttgtataatctgaaggatcggtacggatcctggatgatttacacatactcaggcttgttctgcgtgaccgttaatccctataagtggctgcctgtctacacgccggaggttgttgcagcatatagggggaaaaagaggtccgaagcccctccccacatcttctccatttctgacaacgcctaccagtatatgctgactgacagggagaaccaaagcattcttattaccggagagtctggggcgggcaagacggtaaatacaaaacgcgtgatacagtactttgcagttattgctgcgattggcgacaggagtaaaaaggatcagagccccggcaagggtactctcgaagatcaaattatccaggctaatcccgccctcgaagcgttcgggaatgccaaaacggtccgcaatgataacagtagcagatttgggaagtttatccgcatccatttcggagcgactggcaaactcgcctcagcagacatcgagacatacctgttggaaaagtcacgagtgatatttcagctcaaagccgagcgggactatcatatcttttaccagatactttctaacaaaaaaccagagcttcttgacatgcttctgatcactaacaatccgtatgactatgcattcatcagtcagggagagaccacggttgcgtcaatagacgatgctgaggagctgatggccacagacaatgccttcgatgtgctcggatttactagcgaggagaagaacagcatgtacaagcttaccggtgctatcatgcacttcggcaatatgaaatttaaactcaagcagcgggaagaacaggcggagccggacggaactgaagaggctgataaaagtgcctacctcatggggcttaactctgccgacttgcttaaaggattgtgtcatccacgggtaaaggtaggtaatgaatatgtaactaagggtcaaaatgtgcaacaagtcatatacgcgactggggctctggcaaaagccgtttacgagagaatgtttaactggatggtcacacgaattaatgccactctggagacaaaacaaccccggcagtactttataggtgtgctggatatcgcaggtttcgaaattttcgatttcaactctttcgagcaactttgtatcaatttcaccaacgagaagctccaacaattttttaaccatcatatgttcgttctcgagcaagaggaatacaaaaaagagggcatcgaatggactttcatcgatttcggtatggatcttcaggcttgtatagatctcatcgagaaaccgatggggattatgagtattctggaggaggaatgtatgttcccgaaagccactgacatgacatttaaggccaagctgttcgataatcacttggggaagtccgccaatttccagaaacctaggaacataaaaggtaagccggaggcgcacttctctcttatccattacgcgggaatcgtcgattataacattattggctggcttcaaaagaacaaggatccgcttaacgaaacagtggtcggcctttatcagaaaagctcactgaaacttcttagtacgctctttgctaattacgctggtgctgacgctccaatcgagaaaggcaagggaaaagctaagaaaggcagtagctttcaaacagtctctgccctgcacagagagaatctcaacaagctcatgaccaatctgcggagcacacatccacattttgtcaggtgcataatccctaatgagacgaaaagtccaggcgtaatggacaatccgctggttatgcaccagctcagatgtaatggagtattggaaggtatacgcatatgtagaaaaggcttcccaaacaggatactgtatggtgacttccgacaacgctatcgaatactgaaccccgcagccattcccgagggacaattcatagacagccgcaagggagcggaaaagcttctttcctccctcgacatagaccataaccagtacaaatttgggcacaccaaagtatttttcaaagcgggccttcttggactgttggaagagatgcgcgatgaaaggctgtcaaggataataaccaggattcaagcgcaatctcggggagtgctcgcgaggatggagtacaagaaattgttggaacggagagattccttgcttgtgattcagtggaatatacgagctttcatgggagtaaaaaactggccttggatgaaactctactttaagataaagccactgctgaagtccgcagagagggagaaggagatggcatcaatgaaggaggaatttacgcgcttgaaagaggctctcgagaagtccgaagctagacggaaggaattggaagagaagatggtatctcttttgcaggagaagaatgatcttcagttgcaagtacaagcggaacaagacaatctcgccgacgcagaggaaaggtgtgatcagttgatcaaaaataaaatccaactggaagccaaggtgaaagaaatgaatgagcgcttggaggacgaagaagagatgaatgcagagttgactgcgaagaagcggaagctcgaagacgaatgcctgtcctacgagaccgaaatcctgaccgtggagtatgggctgctgcccatcggcaagattgtggagaagcggattgaatgcaccgtgtatagcgtggacaacaacggcaacatctacacccagcccgtggctcagtggcacgacaggggcgagcaggaggtgtttgagtattgtctggaggacggcagcctgattagagccaccaaagaccacaagttcatgaccgtggacgggcagatgctgcccattgacgagatttttgagcgggaacttgacctgatgcgggtggacaacctgcccaactgaacgcgtgcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagatgtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagagctcggatcctgagaacttcagggtgagtctatgggac

SEQ ID NO: 9 - DNA, 8716 bp, artificial
  AAV vector
ggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcggtaccggtcgccaccatgatgataaagattgccaccagaaagtatctgggcaagcagaacgtgtatgacatcggcgtggagagagaccacaacttcgccctgaagaacggcttcatcgccagcaactgcagcgagctcaagcgcgacattgacgacctggaattgacccttgccaaagtggaaaaggagaaacatgcaaccgagaataaggtaaagaatctgacagaagagatggcgggacttgacgagattatcgcgaagcttacaaaggagaagaaggcccttcaggaagcgcaccaacaggcccttgacgatcttcaggccgaggaagacaaagtgaacacattgactaaagctaaagtgaagcttgagcaacaggtcgatgacctggaaggctcacttgagcaagagaagaaagtgcgcatggatctcgagcgcgctaaacgcaagctcgagggagatttgaagctgactcaagagtcaattatggaccttgaaaacgacaagcaacagttggatgagagacttaagaagaaggacttcgaactcaacgcacttaatgccaggatcgaagatgaacaagctctcgggtctcagctccaaaagaagcttaaggaactccaggcacgcatagaagaactcgaagaagagttggaagccgagagaacggcgagagctaaagtggaaaaactccgaagcgatttgtccagagagctggaagagatttccgagcggcttgaggaggcgggaggcgcgacgagcgtgcagatcgaaatgaataagaagagagaagctgaatttcaaaaaatgcgccgagaccttgaagaagcaacactccagcacgaagccacagcagctgcgcttcggaagaagcatgctgattctgtcgcagagctgggggagcagatcgacaatctccagcgagtcaaacaaaaactggaaaaggagaaatctgagtttaaattggagcttgacgacgtcacgtctaacatggagcagataataaaggcaaaagcaaaccttgagaaaatgtgccgcactcttgaggaccagatgaacgagcatcgcagtaaggcggaagagactcagagatctgttaacgacttgaccagtcaacgagctaaacttcagacagaaaatggagaactctcccgacaactggacgaaaaggaggccctgatcagccagttgactaggggtaaactcacctacacacagcagcttgaggacttgaagcgccaactcgaagaggaggtgaaagctaaaaacgcactggcacatgcgctgcaatccgcaagacatgactgcgacctcctgcgggaacagtacgaagaagaaactgaggcgaaagctgagctccaacgggtactttctaaggcaaattctgaagttgcacagtggcgaactaagtacgaaaccgatgcgatccaacgcacggaggagttggaggaagctaaaaagaagctcgcacagcgactgcaggaagccgaagaagccgttgaggcagttaatgcaaaatgcagtagtctcgagaagactaaacatagactccagaacgaaatagaggatctgatggtggatgttgaacgctccaatgctgctgcggcagccctcgacaaaaaacagcggaatttcgataagattcttgcggaatggaagcagaaatacgaagagtctcagagtgaactggagagctcccaaaaggaggcccgaagtctgtctaccgaattgtttaaactcaagaacgcgtacgaagaatctctggaacacttggaaacctttaaaagagagaacaaaaacctgcaagaggaaataagtgaccttaccgagcaactggggagctccgggaaaaccatccacgaattggaaaaagtcaggaaacagttggaggcagagaaaatggagctccaatccgccctggaggaagcggaagcatctctggaacacgaggagggcaagattctgagggcccaactggaatttaaccagatcaaggcagagatagaacgaaaactcgcggagaaggacgaagagatggagcaggcgaaacggaatcatctgcgggttgtagactccctgcaaacctccctcgatgccgaaaccagaagccggaatgaggcccttcgggtgaaaaaaaaaatggagggcgacttgaacgaaatggaaattcaactttctcacgccaaccgcatggcggccgaggcgcagaaacaggtaaaatctctccagtctctcctcaaagatacacaaatccaactcgacgatgctgtaagggcaaacgatgatttgaaagagaatatagcaatcgtcgagcgccgcaataatctgttgcaagcagagcttgaagaactgcgcgcggtcgtagaacagaccgaacgcagtagaaagttggctgagcaggaacttattgagacttccgagcgcgttcagcttctgcattcccagaacacctctctgattaatcagaagaaaaagatggacgcagatctgtctcagttgcagacggaagtggaagaagctgttcaagagtgccggaacgcggaggaaaaagctaaaaaggcgataactgatgcagcgatgatggctgaggaactgaagaaggagcaagacacctccgcccatttggagcgaatgaagaagaatatggaacaaactattaaggatctccagcaccggcttgatgaggctgaacagatcgccttgaaaggggggaaaaagcaattgcagaaattggaagcccgagtaagggaactggagaatgagttggaagctgagcaaaagcggaacgctgagtctgtgaagggaatgagaaagagtgaacggaggataaaagaactcacatatcaaacggaagaggaccggaaaaacttgctccgcctccaagacctcgttgacaagcttcaattgaaagtcaaggcttataaaagacaagctgaagaagctgaagaacaagcgaacaccaatttgtccaaatttcggaaagtacagcatgagctcgacgaggctgaggagcgggctgacatagctgagtcccaggtcaataaactgcgagcgaaaagcagagacattggcaccaaaggtttgaatgaagagaccggtgagggcagaggaagtcttctaacatgcggtgacgtggaggagaatcccggcccttccggtatgaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaataagcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagatgtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagagctcggatcctgagaacttcagggtgagtctatgggac

SEQ ID NO: 10 - DNA, 7652 bp, artificial
  AAV vector
ggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcggtaccggtcgccaccatggactacaaagaccatgacggtgattataaagatcatgacatcgattacaaggatgacgatgacaagcttgagctcggatccatgggagattctgaaatggccgtcttcggcgctgccgcaccatatctccgcaaaagtgaaaaggagcgcctggaggcccaaaccagacccttcgatctgaaaaaggatgtgtttgtcccggatgacaagcaagaatttgtaaaagcaaagatcgtctctcgggagggcggtaaagtgactgctgaaacagaatacggcaaaaccgtcactgtcaaagaagatcaggtaatgcaacaaaatcctccgaaatttgataaaatcgaagacatggccatgttgacgtttttgcatgagccagctgttttgtataatctgaaggatcggtacggatcctggatgatttacacatactcaggcttgttctgcgtgaccgttaatccctataagtggctgcctgtctacacgccggaggttgttgcagcatatagggggaaaaagaggtccgaagcccctccccacatcttctccatttctgacaacgcctaccagtatatgctgactgacagggagaaccaaagcattcttattaccggagagtctggggcgggcaagacggtaaatacaaaacgcgtgatacagtactttgcagttattgctgcgattggcgacaggagtaaaaaggatcagagccccggcaagggtactctcgaagatcaaattatccaggctaatcccgccctcgaagcgttcgggaatgccaaaacggtccgcaatgataacagtagcagatttgggaagtttatccgcatccatttcggagcgactggcaaactcgcctcagcagacatcgagacatacctgttggaaaagtcacgagtgatatttcagctcaaagccgagcgggactatcatatcttttaccagatactttctaacaaaaaaccagagcttcttgacatgcttctgatcactaacaatccgtatgactatgcattcatcagtcagggagagaccacggttgcgtcaatagacgatgctgaggagctgatggccacagacaatgccttcgatgtgctcggatttactagcgaggagaagaacagcatgtacaagcttaccggtgctatcatgcacttcggcaatatgaaatttaaactcaagcagcgggaagaacaggcggagccggacggaactgaagaggctgataaaagtgcctacctcatggggcttaactctgccgacttgcttaaaggattgtgtcatccacgggtaaaggtaggtaatgaatatgtaactaagggtcaaaatgtgcaacaagtcatatacgcgactggggctctggcaaaagccgtttacgagagaatgtttaactggatggtcacacgaattaatgccactctggagacaaaacaaccccggcagtactttataggtgtgctggatatcgcaggtttcgaaattttcgatttcaactctttcgagcaactttgtatcaatttcaccaacgagaagctccaacaattttttaaccatcatatgttcgttctcgagcaagaggaatacaaaaaagagggcatcgaatggactttcatcgatttcggtatggatcttcaggcttgtatagatctcatcgagaaaccgatggggattatgagtattctggaggaggaatgtatgttcccgaaagccactgacatgacatttaaggccaagctgttcgataatcacttggggaagtccgccaatttccagaaacctaggaacataaaaggtaagccggaggcgcacttctctcttatccattacgcgggaatcgtcgattataacattattggctggcttcaaaagaacaaggatccgcttaacgaaacagtggtcggcctttatcagaaaagctcactgaaacttcttagtacgctctttgctaattacgctggtgctgacgctccaatcgagaaaggcaagggaaaagctaagaaaggcagtagctttcaaacagtctctgccctgcacagagagaatctcaacaagctcatgaccaatctgcggagcacacatccacattttgtcaggtgcataatccctaatgagacgaaaagtccaggcgtaatggacaatccgctggttatgcaccagctcagatgtaatggagtattggaaggtatacgcatatgtagaaaaggcttcccaaacaggatactgtatggtgacttccgacaacggtatcgcatcctgaacccagcggccatccctgagggacagttcattgatagcaggaagggggcagagaagctgctcagctccctggacattgatcacaaccagtacaagtttggccacaccaaggtgagtaaaggagactaattaattaaaggaagacatctctgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagatgtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagagctcggatcctgagaacttcagggtgagtctatgggac

SEQ ID NO: 11 - DNA, 8587 bp, artificial
  AAV vector
ggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctacccctccctagtcatggccaacacacaccttgcctgcaggtgttcttcaaggccgggctgctggggctgctggaggaaatgagggacgagaggctgagccgcatcatcacgcgtatccaggcccagtcccgaggtgtgctcgccagaatggagtacaaaaagctgctggaacgtagagattccttgcttgtgattcagtggaatatacgagctttcatgggagtaaaaaactggccttggatgaaactctactttaagataaagccactgctgaagtccgcagagagggagaaggagatggcatcaatgaaggaggaatttacgcgcttgaaagaggctctcgagaagtccgaagctagacggaaggaattggaagagaagatggtatctcttttgcaggagaagaatgatcttcagttgcaagtacaagcggaacaagacaatctcgccgacgcagaggaaaggtgtgatcagttgatcaaaaataaaatccaactggaagccaaggtgaaagaaatgaatgagcgcttggaggacgaagaagagatgaatgcagagttgactgcgaagaagcggaagctcgaagacgaatgcagcgagctcaagcgcgacattgacgacctggaattgacccttgccaaagtggaaaaggagaaacatgcaaccgagaataaggtaaagaatctgacagaagagatggcgggacttgacgagattatcgcgaagcttacaaaggagaagaaggcccttcaggaagcgcaccaacaggcccttgacgatcttcaggccgaggaagacaaagtgaacacattgactaaagctaaagtgaagcttgagcaacaggtcgatgacctggaaggctcacttgagcaagagaagaaagtgcgcatggatctcgagcgcgctaaacgcaagctcgagggagatttgaagctgactcaagagtcaattatggaccttgaaaacgacaagcaacagttggatgagagacttaagaagaaggacttcgaactcaacgcacttaatgccaggatcgaagatgaacaagctctcgggtctcagctccaaaagaagcttaaggaactccaggcacgcatagaagaactcgaagaagagttggaagccgagagaacggcgagagctaaagtggaaaaactccgaagcgatttgtccagagagctggaagagatttccgagcggcttgaggaggcgggaggcgcgacgagcgtgcagatcgaaatgaataagaagagagaagctgaatttcaaaaaatgcgccgagaccttgaagaagcaacactccagcacgaagccacagcagctgcgcttcggaagaagcatgctgattctgtcgcagagctgggggagcagatcgacaatctccagcgagtcaaacaaaaactggaaaaggagaaatctgagtttaaattggagcttgacgacgtcacgtctaacatggagcagataataaaggcaaaagcaaaccttgagaaaatgtgccgcactcttgaggaccagatgaacgagcatcgcagtaaggcggaagagactcagagatctgttaacgacttgaccagtcaacgagctaaacttcagacagaaaatggagaactctcccgacaactggacgaaaaggaggccctgatcagccagttgactaggggtaaactcacctacacacagcagcttgaggacttgaagcgccaactcgaagaggaggtgaaagctaaaaacgcactggcacatgcgctgcaatccgcaagacatgactgcgacctcctgcgggaacagtacgaagaagaaactgaggcgaaagctgagctccaacgggtactttctaaggcaaattctgaagttgcacagtggcgaactaagtacgaaaccgatgcgatccaacgcacggaggagttggaggaagctaaaaagaagctcgcacagcgactgcaggaagccgaagaagccgttgaggcagttaatgcaaaatgcagtagtctcgagaagactaaacatagactccagaacgaaatagaggatctgatggtggatgttgaacgctccaatgctgctgcggcagccctcgacaaaaaacagcggaatttcgataagattcttgcggaatggaagcagaaatacgaagagtctcagagtgaactggagagctcccaaaaggaggcccgaagtctgtctaccgaattgtttaaactcaagaacgcgtacgaagaatctctggaacacttggaaacctttaaaagagagaacaaaaacctgcaagaggaaataagtgaccttaccgagcaactggggagctccgggaaaaccatccacgaattggaaaaagtcaggaaacagttggaggcagagaaaatggagctccaatccgccctggaggaagcggaagcatctctggaacacgaggagggcaagattctgagggcccaactggaatttaaccagatcaaggcagagatagaacgaaaactcgcggagaaggacgaagagatggagcaggcgaaacggaatcatctgcgggttgtagactccctgcaaacctccctcgatgccgaaaccagaagccggaatgaggcccttcgggtgaaaaaaaaaatggagggcgacttgaacgaaatggaaattcaactttctcacgccaaccgcatggcggccgaggcgcagaaacaggtaaaatctctccagtctctcctcaaagatacacaaatccaactcgacgatgctgtaagggcaaacgatgatttgaaagagaatatagcaatcgtcgagcgccgcaataatctgttgcaagcagagcttgaagaactgcgcgcggtcgtagaacagaccgaacgcagtagaaagttggctgagcaggaacttattgagacttccgagcgcgttcagcttctgcattcccagaacacctctctgattaatcagaagaaaaagatggacgcagatctgtctcagttgcagacggaagtggaagaagctgttcaagagtgccggaacgcggaggaaaaagctaaaaaggcgataactgatgcagcgatgatggctgaggaactgaagaaggagcaagacacctccgcccatttggagcgaatgaagaagaatatggaacaaactattaaggatctccagcaccggcttgatgaggctgaacagatcgccttgaaaggggggaaaaagcaattgcagaaattggaagcccgagtaagggaactggagaatgagttggaagctgagcaaaagcggaacgctgagtctgtgaagggaatgagaaagagtgaacggaggataaaagaactcacatatcaaacggaagaggaccggaaaaacttgctccgcctccaagacctcgttgacaagcttcaattgaaagtcaaggcttataaaagacaagctgaagaagctgaagaacaagcgaacaccaatttgtccaaatttcggaaagtacagcatgagctcgacgaggctgaggagcgggctgacatagctgagtcccaggtcaataaactgcgagcgaaaagcagagacattggcaccaaaggtttgaatgaagaggagggcagaggaagtcttctaacatgcggtgacgtggaggagaatcccggccctatgaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaataagc

(19) United States
(12) Patent Application Publication
(10) Pub. No.: US 2023/0047424 A1
(43) Pub. Date: Feb. 16, 2023

(54) GENE THERAPY COMPOSITION AND TREATMENT FOR MYH7-LINKED CARDIOMYOPATHY

(71) Applicant: UCL BUSINESS LTD, London (GB)

(72) Inventors: Thomas Voit, London (GB); Julie Dumonceaux, London (GB); Virginie Mariot, London (GB)

(21) Appl. No.: 17/783,167

(22) PCT Filed: Dec. 9, 2020
(86) PCT No.: PCT/EP2020/085158

§ 371 Date: Jun. 7, 2022

Related U.S. Application Data
(60) Provisional application No. 62/945,518, filed on Dec. 9, 2019.

(51) Int. Cl. A61K 48/00; C12N 15/86

(52) U.S. Cl. CPC A61K 48/0058; C12N 15/86; C12N 2750/14143

(57) ABSTRACT

Disclosed are a composition and method of treating or preventing cardiomyopathy in a human subject. In one embodiment, a method comprises delivering a gene therapy drug to cardiac tissue of the human subject. The gene therapy drug comprises: a first vector comprising a first portion of a polynucleotide sequence encoding for a therapeutic protein; and a second vector comprising a second portion of the polynucleotide sequence encoding for the therapeutic protein.

### CROSS-REFERENCE TO RELATED APPLICATION(S)

This application claims the benefit of priority of U.S. Provisional Patent Application Ser. No. 62/945,518, filed on Dec. 9, 2019, the disclosure of which is hereby incorporated by reference herein in its entirety.

### FIELD OF THE INVENTION

The present invention relates to the treatment of cardiac diseases (e.g., cardiac myopathies), and, more specifically, to gene therapy methods and pharmaceutical compositions for the treatment of hypertrophic cardiomyopathy.

### BACKGROUND OF THE INVENTION

Despite pharmacologic advances in the treatment of various heart conditions, such as heart failure, mortality, and morbidity remain unacceptably high. Furthermore, certain therapeutic approaches are not suitable for many patients (e.g., ones who have an advanced heart failure condition associated with other co-morbid diseases). Alternative approaches, such as gene therapy and cell therapy, have attracted increased attention due to their potential to be uniquely tailored and efficacious in addressing the root cause pathogenesis of many cardiac diseases.

### OBJECTS AND SUMMARY OF THE INVENTION

It is an object of certain embodiments of the present invention to provide methods of delivering therapeutic polynucleotide sequences to cardiomyocytes of a human subject.

It is a further object of certain embodiments of the present invention to utilize gene therapy methods for treating MYH7-linked cardiomyopathy.

It is a further object of certain embodiments of the present invention to vectorize a polynucleotide sequence encoding for MYH7.

It is a further object of certain embodiments to split a gene into two or more vectors that are delivered to cardiac tissue of a patient for scenarios in which the gene size exceeds the packaging capacity of the vector.

The above objects and others are met by the present invention, which in certain embodiments is directed to a method of treating or preventing cardiomyopathy in a human subject. In one aspect, the method comprises delivering a gene therapy drug to cardiac tissue of the human subject. The gene therapy drug comprises a first vector comprising a first portion of a polynucleotide sequence encoding for a therapeutic protein; and a second vector comprising a second portion of the polynucleotide sequence encoding for the therapeutic protein.

In some embodiments, the first portion and the second portion of the polynucleotide sequence collectively define the entire polynucleotide sequence from its 5′ end to its 3′ end. The first portion may comprise a first continuous sequence starting from the 5′ end and ending upstream from the 3′ end, and the second portion may comprise a second continuous sequence starting downstream from the 5′ end and ending at the 3′ end. In some embodiments, the first continuous sequence comprises a first overlap portion, the second continuous sequence comprises a second overlap portion, the first overlap portion overlaps with the second overlap portion, and the first overlap portion and the second overlap portion are single-stranded and non-complementary to each other.

In some embodiments, the therapeutic protein comprises a functional MYH7 protein, and wherein the polynucleotide sequence encodes for the functional MYH7 protein. In some embodiments, the first portion of the polynucleotide sequence comprises less than about half of the polynucleotide sequence starting from the 5′ end, and the second portion of the polynucleotide sequence comprises a remainder of the polynucleotide sequence. In some embodiments, the first portion of the polynucleotide sequence comprises more than about half of the polynucleotide sequence starting from the 5′ end, and the second portion of the polynucleotide sequence comprises a remainder of the polynucleotide sequence. In some embodiments, the first portion and the second portion of the polynucleotide sequence collectively define the polynucleotide sequence, the first portion comprises a first continuous sequence starting from the 5′ end and ending upstream from the 3′ end, the second portion comprises a second continuous sequence starting downstream from the 5′ end and ending at 3′ end, and both the first continuous sequence and the second continuous sequence are single-stranded and non-complementary to each other.

In some embodiments, the first continuous sequence comprises a first overlap portion, the second continuous sequence comprises a second overlap portion, and the first overlap portion overlaps with the second overlap portion. In some embodiments, the first overlap portion and the second overlap portion are each greater than 10 bases and less than 4,800 bases. In some embodiments, the first overlap portion and the second overlap portion encode for intron 20 of the polynucleotide sequence. In some embodiments, the first continuous sequence comprises exons 1 to 27 of the polynucleotide sequence, the second continuous sequence comprises exons 19 to 40 of the polynucleotide sequence, and the first overlap portion and the second overlap portion each comprises exons 19 to 27 of the polynucleotide sequence.

In some embodiments, the first vector further comprises a cardiac muscle-specific promotor. In some embodiments, the first vector further comprises a chimeric intron. In some embodiments, each of the first vector and the second vector comprises a viral vector. In some embodiments, one or more of the first vector or the second vector comprises one or more adeno-associated viral (AAV) vectors. In some embodiments, one or more of the first vector or the second vector comprises rAAV2/9.

In another aspect, a viral vector comprises less than an entire sequence of a polynucleotide sequence encoding for a functional MYH7 protein.

In another aspect, a method of treating or preventing hypertrophic cardiomyopathy in a human subject comprises delivering a gene therapy drug to cardiac tissue of the human subject. The gene therapy drug comprises: a first rAAV2/9 vector comprising a continuous first portion of less than all of a polynucleotide sequence encoding for a functional MYH7 protein starting from the 5′ end and ending upstream from the 3′ end; and a second rAAV2/9 vector comprising a continuous second portion of less than all of the polynucleotide sequence starting downstream from the 5′ end and ending at the 3′ end.

In another aspect, a method of treating or preventing hypertrophic cardiomyopathy in a human subject comprises delivering a first rAAV2/9 vector comprising a continuous first portion of less than all of a polynucleotide sequence encoding for a functional MYH7 protein starting from the 5′ end and ending upstream from the 3′ end. In some embodiments, the method further comprises delivering a second rAAV2/9 vector comprising a continuous second portion of less than all of the polynucleotide sequence starting downstream from the 5′ end and ending at the 3′ end.

### BRIEF DESCRIPTION OF THE DRAWINGS

The above and other features of the present disclosure, their nature, and various advantages will become more apparent upon consideration of the following detailed description, taken in conjunction with the accompanying drawings, in which:

FIG. 1A is a schematic illustrating two vectors sharing homologous overlapping sequences in accordance with at least one embodiment;

FIG. 1B is a vector map illustrating a vector encoding a first portion of an MYH7 coding region in accordance with at least one embodiment;

FIG. 1C is a vector map illustrating a vector encoding a second portion of the MYH7 coding region in accordance with at least one embodiment;

FIG. 1D is a schematic illustrating the overlap region of vectors encoding for portions of MYH7 in accordance with at least one embodiment;

FIG. 2A is a schematic illustrating protein splicing based on encoded intein sequences in accordance with at least one embodiment;

FIG. 2B is a vector map illustrating a vector encoding a first portion of an MYH7 coding region and an N-intein sequence in accordance with at least one embodiment.

FIG. 2C is a vector map illustrating a vector encoding a second portion of the MYH7 coding region and a C-intein sequence in accordance with at least one embodiment; and

FIG. 3 is a schematic illustrating splicing of two nucleic acid coding sequences through concatemerization or homologous recombination in accordance with at least one embodiment.

### DEFINITIONS

As used herein, the singular forms “a,” “an,” and “the” include plural references unless the context clearly indicates otherwise. Thus, for example, reference to “a drug” includes a single drug as well as a mixture of two or more different drugs; and reference to a “viral vector” includes a single viral vector as well as a mixture of two or more different viral vectors, and the like.

Also as used herein, “about,” when used in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number±10%, such that “about 10” would include from 9 to 11.

Also as used herein, “polynucleotide” has its ordinary and customary meaning in the art and includes any polymeric nucleic acid such as DNA or RNA molecules, as well as chemical derivatives known to those skilled in the art. Polynucleotides include not only those encoding a therapeutic protein, but also include sequences that can be used to decrease the expression of a targeted nucleic acid sequence using techniques known in the art (e.g., antisense, interfering, or small interfering nucleic acids). Polynucleotides can also be used to initiate or increase the expression of a targeted nucleic acid sequence or the production of a targeted protein within cells of the cardiovascular system. Targeted nucleic acids and proteins include, but are not limited to, nucleic acids and proteins normally found in the targeted tissue, derivatives of such naturally occurring nucleic acids or proteins, naturally occurring nucleic acids or proteins not normally found in the targeted tissue, or synthetic nucleic acids or proteins. One or more polynucleotides can be used in combination, administered simultaneously and/or sequentially, to increase and/or decrease one or more targeted nucleic acid sequences or proteins.

Also as used herein, “exogenous” nucleic acids or genes are those that do not occur in nature in the vector utilized for nucleic acid transfer; e.g., not naturally found in the viral vector, but the term is not intended to exclude nucleic acids encoding a protein or polypeptide that occurs naturally in the patient or host.

Also as used herein, “cardiac cell” includes any cell of the heart that is involved in maintaining a structure or providing a function of the heart such as a cardiac muscle cell, a cell of the cardiac vasculature, or a cell present in a cardiac valve. Cardiac cells include cardio myocytes (having both normal and abnormal electrical properties), epithelial cells, endothelial cells, fibroblasts, cells of the conducting tissue, cardiac pace making cells, and neurons.

Also as used herein, “adeno-associated virus” or “AAV” encompasses all subtypes, serotypes and pseudotypes, as well as naturally occurring and recombinant forms. A variety of AAV serotypes and strains are known in the art and are publicly available from sources, such as the ATCC, and academic or commercial sources. Alternatively, sequences from AAV serotypes and strains which are published and/or available from a variety of databases may be synthesized using known techniques.

Also as used herein, “serotype” refers to an AAV which is identified by and distinguished from other AAVs based on capsid protein reactivity with defined antisera. There are at least twelve known serotypes of human AAV, including AAV1 through AAV12, however additional serotypes continue to be discovered, and use of newly discovered serotypes are contemplated.

Also as used herein, “pseudotyped” AAV refers to an AAV that contains capsid proteins from one serotype and a viral genome including 5′ and 3′ inverted terminal repeats (ITRs) of a different or heterologous serotype. A pseudotyped recombinant AAV (rAAV) would be expected to have cell surface binding properties of the capsid serotype and genetic properties consistent with the ITR serotype. A pseudotyped rAAV may comprise AAV capsid proteins, including VP1, VP2, and VP3 capsid proteins, and ITRs from any serotype AAV, including any primate AAV serotype from AAV1 through AAV12, as long as the capsid protein is of a serotype heterologous to the serotype(s) of the ITRs. In a pseudotyped rAAV, the 5′ and 3′ ITRs may be identical or heterologous. Pseudotyped rAAV are produced using standard techniques described in the art.

Also as used herein, “chimeric” rAAV vector encompasses an AAV vector comprising heterologous capsid proteins; that is, a rAAV vector may be chimeric with respect to its capsid proteins VP1, VP2, and VP3, such that VP1, VP2, and VP3 are not all of the same serotype AAV. A chimeric AAV as used herein encompasses AAV wherein the capsid proteins VP1, VP2, and VP3 differ in serotypes, including for example but not limited to capsid proteins from AAV1 and AAV2; are mixtures of other parvo virus capsid proteins or comprise other virus proteins or other proteins, such as for example, proteins that target delivery of the AAV to desired cells or tissues. A chimeric rAAV as used herein also encompasses an rAAV comprising chimeric 5′ and 3′ ITRs.

Also as used herein, a “pharmaceutically acceptable excipient or carrier” refers to any inert ingredient in a composition that is combined with an active agent in a formulation. A pharmaceutically acceptable excipient can include, but is not limited to, carbohydrates (such as glucose, sucrose, or dextrans), antioxidants (such as ascorbic acid or glutathione), chelating agents, low-molecular weight proteins, high-molecular weight polymers, gel-forming agents, or other stabilizers and additives. Other examples of a pharmaceutically acceptable carrier include wetting agents, emulsifying agents, dispersing agents, or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

Also as used herein, a “patient” refers to a subject, particularly a human (but could also encompass a non-human), who has presented a clinical manifestation of a particular symptom or symptoms suggesting the need for treatment, who is treated prophylactically for a condition, or who has been diagnosed with a condition to be treated.

Also as used herein, a “subject” encompasses the definition of the term “patient” and does not exclude individuals who are otherwise healthy.

Also as used herein, “treatment of” and “treating” include the administration of a drug with the intent to lessen the severity of or prevent a condition, e.g., heart disease.

Also as used herein, “prevention of” and “preventing” include the avoidance of the onset of a condition, e.g., heart disease.

Also as used herein, a “condition” or “conditions” refers to those medical conditions, such as heart disease, that can be treated, mitigated, or prevented by administration to a subject of an effective amount of a drug.

Also as used herein, an “effective amount” refers to the amount of a drug that is sufficient to produce a beneficial or desired effect at a level that is readily detectable by a method commonly used for detection of such an effect. In some embodiments, such an effect results in a change of at least 10% from the value of a basal level where the drug is not administered. In other embodiments, the change is at least 20%, 50%, 80%, or an even higher percentage from the basal level. As will be described below, the effective amount of a drug may vary from subject to subject, depending on age, general condition of the subject, the severity of the condition being treated, the particular drug administered, and the like. An appropriate “effective” amount in any individual case may be determined by one of ordinary skill in the art by reference to the pertinent texts and literature and/or by using routine experimentation.

Also as used herein, an “active agent” refers to any material that is intended to produce a therapeutic, prophylactic, or other intended effect, whether or not approved by a government agency for that purpose.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

### DETAILED DESCRIPTION

Hypertrophic cardiomyopathy (HMC) is the most common inherited cardiovascular disease with a prevalence of 1 in 500 adults, and is characterized by an increased wall thickness of the left ventricle. HMC is a highly complex and heterogenous disease in its clinical variations, ranging from asymptomatic status to heart failure.

HMC is accepted as a disease of the sarcomere, which is responsible for generating the molecular force of cardiomyocyte contraction by converting chemical energy of ATP hydrolysis. Seventy percent of genetic HCM cases carry mutations in 1 of 8 sarcomeric protein genes, mainly MYBPC3 and MYH7 variants, and MYH7 mutations are responsible for approximately 40% of genetic HCM cases. Importantly, childhood cases with severe HCM or a combination of HCM and dilated cardiomyopathy are not uncommon. While therapeutic strategies have been proposed for MYBPC3, no work has been done concerning MYH7 despite the fact that the presence of mutations in this gene is associated with a poor prognosis.

The present invention relates to an AAV-based approach to treat MYH7-related cardiomyopathies. The MYH7 gene is located on chromosome 14 and encodes a class II myosin expressed in slow, type 1 muscle fibers as well as in the heart muscle. Both cardiac and skeletal muscle disorders can arise from mutations in MYH7, but cardiac disease is more frequent with more than 320 mutations having been identified. MYH7 is a 23 kilobase (kb) long gene, composed of 40 exons forming one transcript of 6087 bases (NCBI Gene ID: 4625), which encodes the 1935 amino acid MYH7 protein (SEQ ID NO: 1). The protein is composed of two regions: a head and a tail. The globular head region, called the motor domain, binds to actin and ATP and is located in the N-terminal portion. The long tail region (also called the ROD domain or the light meromyosin domain-LMM) is located in the C-terminal portion and is essential for the protein dimerization and interaction with other proteins including titin, myosin-binding protein C3, myomesin-1, etc. Mutations accounting for the cardiac or skeletal muscle disorders cluster in different parts of the protein. Most cardiomyopathy related mutations being located in the globular head domain potentially affecting the binding sites for actin, while mutations linked to skeletal myopathy are usually located in the distal regions of the ROD domain.

At present, for all inherited diseases and heart failure, the only curative treatment is heart transplantation. Cardiac gene therapy with AAV-based vectors holds great promise for the treatment of MYH7-linked HCM. AAV vectors are non-pathogenic, unable to replicate on their own, persist in the host nucleus in an extra-chromosomal form, and can be delivered by intra-myocardial or intracoronary or systemic injections. AAV vectors, which have a limited packaging capacity of approximately 5 kb, have been successfully used for transgenes exceeding 5 kb by splitting the corresponding polynucleotide sequence into 2 components, whereby the 5′ component and the 3′ component overlap significantly, usually for approximately 1000 bases, to allow for cDNA concatemerization after delivery via two AAV vectors. AAV vectors have previously been used to treat HCM using a Mybpc3-targeted knock-in (KI) mouse model in vivo.

Certain embodiments of the present disclosure relate to different approaches involving a combination of two or more AAVs in connection with AAV-mediated MYH7 gene expression in cardiomyocytes (e.g., hiPSC-derived cardiomyocytes).

In one embodiment, a first vector (e.g., a 5′ cassette) comprises a cardiac muscle-specific promoter (such as TNNT2), and a first portion (e.g., approximately half) of a polynucleotide sequence encoding for MYH7. The first vector may also include a chimeric intron to enhance transcription of the first portion of the polynucleotide sequence. Each of the two vectors may be single stranded polynucleotide sequences.

In another embodiment, the first portion of the polynucleotide sequence has a subportion that overlaps with overlaps with a subportion of the second portion of the polynucleotide sequence. For example, the polynucleotide sequence of the first vector may include a continuous sequence starting at the 5′ end of the MYH7 sequence that includes up to and including intron 23 of the MYH7 polynucleotide sequence. The polynucleotide sequence of the second vector may start from the 5′ end of intron 23 and continue continuously to the 3′ end of the MYH7 polynucleotide sequence. This particular example results in a 183 base overlap of the sequences from the two cassettes, with each being single stranded and non-complementary. In other embodiments, the overlap may be based on a different intron, such as intron 20.

In another embodiment, the first portion in the first vector corresponds to exons 1 to 27 of the MYH7 polynucleotide sequence, and the second portion in the second vector corresponds to exons 19 to 40 of the MYH7 polynucleotide sequence, thus exhibiting an overlap of 1682 bases.

It is noted that these embodiments are exemplary, and other overlaps are also contemplated. For example, it is contemplated that other ranges of exons may be spanned by each portion of the MYH7 polynucleotide sequence split across the two vectors. For example, the first vector may contain exons 1 to 35, exons 1 to 34, exons 1 to 33, etc., and similarly the second vector may contain exons 15 to 40, 16 to 40, 17 to 40, etc. Each vector may contain any range of exons provided that the number of bases per portion of the polynucleotide sequence is of a size capable of being packaged into its viral particle (e.g., less than approximately 5 kb for AAV).

In some embodiments, the first overlap portion and the second overlap portion are each greater than 10 bases and less than 4,800 bases. In some embodiments, the overlap portions may be 10 bases, 4,800 bases, or any integer number therebetween (e.g., 100 bases, 200 bases, etc.). Suitable subranges within 10 to 4,800 bases are also contemplated (e.g., 100 to 4,800, 200 to 4,800, 100 to 4,500, etc.). Moreover, embodiments utilizing more than two vectors are contemplated (e.g., the MYH7 polynucleotide sequence may be split into three separate vectors).

An “intein” is a segment of a protein capable of excising itself and joining the remaining portions (referred to as “exteins”) with a peptide bond in a process termed protein splicing. Inteins are also referred to as “protein introns.” In some embodiments, the first vector comprises a polynucleotide sequence encoding for a first protein fragment and the second vector comprises a second polynucleotide sequence encoding for a second protein fragment. The first protein fragment comprises an N-terminal MYH7 fragment having an N-intein sequence at its C-terminus, and the second protein fragment comprises a C-terminal MYH7 fragment having a C-intein sequence at its N-terminus. After the polynucleotide sequences are expressed as their respective protein fragments, the N-intein and C-intein recognize each other and self-catalyze a reaction that ligates their respective flanking MYH7 fragments, resulting in a fully-formed and functional MYH7 protein.

In some embodiments, each cassette is packaged into a suitable AAV. For example, the cassettes may each be packaged into rAAV2/9, which is a particularly efficient serotype for cardiomyocyte transduction.

Although numerous embodiments herein are described with respect to MYH7 protein, it is to be understood that the expression of additional proteins (e.g., sarcomeric proteins) is contemplated. Exemplary proteins include in addition to MYH7, without limitations, one or more of PKP2, SERCA2, MYBPC3, MYL3, MYL2, ACTC1, TPM1, TNNT2, TNNI3, TTN, FHL1, ALPK3, dystrophin, FKRP, variants thereof, or combinations thereof. The protein or proteins used may also be functional variants of the proteins mentioned herein and may exhibit a significant amino acid sequence identity compared to the original protein. For instance, the amino acid identity may amount to at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%. In this context, the term “functional variant” means that the variant of the protein is capable of, partially or completely, fulfilling the function of the naturally occurring corresponding protein. Functional variants of a protein may include, for example, proteins that differ from their naturally occurring counterparts by one or more amino acid substitutions, deletions, or additions.

The amino acid substitutions can be conservative or non-conservative. It is preferred that the substitutions are conservative substitutions, i.e., a substitution of an amino acid residue by an amino acid of similar polarity, which acts as a functional equivalent. Preferably, the amino acid residue used as a substitute is selected from the same group of amino acids as the amino acid residue to be substituted. For example, a hydrophobic residue can be substituted with another hydrophobic residue, or a polar residue can be substituted with another polar residue having the same charge. Functionally homologous amino acids, which may be used for a conservative substitution comprise, for example, non-polar amino acids such as glycine, valine, alanine, isoleucine, leucine, methionine, proline, phenylalanine, and tryptophan. Examples of uncharged polar amino acids comprise serine, threonine, glutamine, asparagine, tyrosine and cysteine. Examples of charged polar (basic) amino acids comprise histidine, arginine, and lysine. Examples of charged polar (acidic) amino acids comprise aspartic acid and glutamic acid.

Also considered as variants are proteins that differ from their naturally occurring counterparts by one or more (e.g., 2, 3, 4, 5, 10, or 15) additional amino acids. These additional amino acids may be present within the amino acid sequence of the original protein (i.e., as an insertion), or they may be added to one or both termini of the protein. Basically, insertions can take place at any position if the addition of amino acids does not impair the capability of the polypeptide to fulfill the function of the naturally occurring protein in the treated subject. Moreover, variants of proteins also comprise proteins in which, compared to the original polypeptide, one or more amino acids are lacking. Such deletions may affect any amino acid position provided that it does not impair the ability to fulfill the normal function of the protein.

Finally, variants of cardiac sarcomeric proteins (e.g., MYH7) also refer to proteins that differ from the naturally occurring protein by structural modifications, such as modified amino acids. Modified amino acids are amino acids which have been modified either by natural processes, such as processing or post-translational modifications, or by chemical modification processes known in the art. Typical amino acid modifications comprise phosphorylation, glycosylation, acetylation, O-Linked N-acetylglucosamination, glutathionylation, acylation, branching, ADP ribosylation, crosslinking, disulfide bridge formation, formylation, hydroxylation, carboxylation, methylation, demethylation, amidation, cyclization, and/or covalent or non-covalent bonding to phosphotidylinositol, flavine derivatives, lipoteichonic acids, fatty acids, or lipids.

The therapeutic polynucleotide sequence encoding the target protein may be administered to the subject to be treated in the form of a gene therapy vector, i.e., a nucleic acid construct which comprises the coding sequence, including the translation and termination codons, next to other sequences required for providing expression of the exogenous nucleic acid such as promoters, kozak sequences, polyA signals and the like.

For example, the gene therapy vector may be part of a mammalian expression system. Useful mammalian expression systems and expression constructs are commercially available. Also, several mammalian expression systems are distributed by different manufacturers and can be employed in the present invention, such as plasmid- or viral vector based systems, e.g., LENTI-Smart™ (InvivoGen), GenScript™ Expression vectors, pAdVAntage™ (Promega), ViraPower™ Lentiviral, Adenoviral Expression Systems (Invitrogen), and adeno-associated viral expression systems (Cell Biolabs).

Gene therapy vectors for expressing an exogenous therapeutic polynucleotide sequence of the invention can be, for example, a viral or non-viral expression vector, which is suitable for introducing the exogenous therapeutic polynucleotide sequence into a cell for subsequent expression of the protein encoded by said nucleic acid. The expression vector can be an episomal vector, i.e., one that is capable of self-replicating autonomously within the host cell, or an integrating vector, i.e., one which stably incorporates into the genome of the cell. The expression in the host cell can be constitutive or regulated (e.g., inducible).

In a certain embodiment, the gene therapy vector is a viral expression vector. Viral vectors for use in the present invention may comprise a viral genome in which a portion of the native sequence has been deleted in order to introduce a heterogeneous polynucleotide without destroying the infectivity of the virus. Due to the specific interaction between virus components and host cell receptors, viral vectors are highly suitable for efficient transfer of genes into target cells. Suitable viral vectors for facilitating gene transfer into a mammalian cell can be derived from different types of viruses, for example, from an AAV, an adenovirus, a retrovirus, a herpes simplex virus, a bovine papilloma virus, a lentivirus, a vaccinia virus, a polyoma virus, a sendai virus, orthomyxovirus, paramyxovirus, papovavirus, picornavirus, pox virus, alphavirus, or any other viral shuttle suitable for gene therapy, variations thereof, and combinations thereof.

“Adenovirus expression vector” or “adenovirus” is meant to include those constructs containing adenovirus sequences sufficient (a) to support packaging of the therapeutic polynucleotide sequence construct, and/or (b) to ultimately express a tissue and/or cell-specific construct that has been cloned therein. In one embodiment of the invention, the expression vector comprises a genetically engineered form of adenovirus. Knowledge of the genetic organization of adenovirus, a 36 kilobase (kb), linear, double-stranded DNA virus, allows substitution of large pieces of adenoviral DNA with foreign sequences up to 7 kb.

Adenovirus growth and manipulation is known to those of skill in the art, and exhibits broad host range in vitro and in vivo. This group of viruses can be obtained in high titers, e.g., 10⁹ to 10¹¹ plaque-forming units per mL, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus, demonstrating their safety and/or therapeutic potential as in vivo gene transfer vectors.

Retroviruses (also referred to as “retroviral vector”) may be chosen as gene delivery vectors due to their ability to integrate their genes into the host genome, transferring a large amount of foreign genetic material, infecting a broad spectrum of species and cell types and for being packaged in special cell-lines.

The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome.

In order to construct a retroviral vector, a nucleic acid encoding a gene of interest is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression require the division of host cells.

The retrovirus can be derived from any of the subfamilies. For example, vectors from Murine Sarcoma Virus, Bovine Leukemia, Virus Rous Sarcoma Virus, Murine Leukemia Virus, Mink-Cell Focus-Inducing Virus, Reticuloendotheliosis Virus, or Avian Leukosis Virus can be used. The skilled person will be able to combine portions derived from different retroviruses, such as LTRs, tRNA binding sites, and packaging signals to provide a recombinant retrovirus. These retroviruses are then normally used for producing transduction competent retroviral vector particles. For this purpose, the vectors are introduced into suitable packaging cell lines. Retroviruses can also be constructed for site-specific integration into the DNA of the host cell by incorporating a chimeric integrase enzyme into the retroviral particle.

Because herpes simplex virus (HSV) is neurotropic, it has generated considerable interest in treating nervous system disorders. Moreover, the ability of HSV to establish latent infections in non-dividing neuronal cells without integrating into the host cell chromosome or otherwise altering the host cell's metabolism, along with the existence of a promoter that is active during latency makes HSV an attractive vector. And though much attention has focused on the neurotropic applications of HSV, this vector also can be exploited for other tissues given its wide host range.

Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient multiplicity of infection (MOI) and in a lessened need for repeat dosing. Avirulent variants of HSV have been developed and are readily available for use in gene therapy contexts.

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV-1, HIV-2) and the Simian Immunodeficiency Virus (SIV). Lentiviral vectors have been generated by multiply attenuating the HIV virulence genes, for example, the genes env, vif, vpr, vpu, and nef are deleted making the vector biologically safe.

Lentiviral vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest.

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most highly conserved among poxviruses. Estimated open reading frames in vaccinia virus number from 150 to 200. Although both strands are coding, extensive overlap of reading frames is not common.

At least 25 kb can be inserted into the vaccinia virus genome. Prototypical vaccinia vectors contain transgenes inserted into the viral thymidine kinase gene via homologous recombination. Vectors are selected on the basis of a tk-phenotype. Inclusion of the untranslated leader sequence of encephalomyocarditis virus results in a level of expression that is higher than that of conventional vectors, with the transgenes accumulating at 10% or more of the infected cell's protein in 24 hours.

The empty capsids of papovaviruses, such as the mouse polyoma virus, have received attention as possible vectors for gene transfer. The use of empty polyoma was first described when polyoma DNA and purified empty capsids were incubated in a cell-free system. The DNA of the new particle was protected from the action of pancreatic DNase. The reconstituted particles were used for transferring a transforming polyoma DNA fragment to rat FIII cells. The empty capsids and reconstituted particles consist of all three of the polyoma capsid antigens VP1, VP2 and VP3.

AAVs are parvoviruses belonging to the genus Dependovirus. They are small, nonenveloped, single-stranded DNA viruses which require a helper virus in order to replicate. Co-infection with a helper virus (e.g., adenovirus, herpes virus, or vaccinia virus) is necessary in order to form functionally complete AAV virions. In vitro, in the absence of co-infection with a helper virus, AAV establishes a latent state in which the viral genome exists in an episomal form, but infectious virions are not produced. Subsequent infection by a helper virus “rescues” the genome, allowing it to be replicated and packaged into viral capsids, thereby reconstituting the infectious virion. Recent data indicate that in vivo both wild type AAV and recombinant AAV predominantly exist as large episomal concatemers. In one embodiment, the gene therapy vector used herein is an AAV vector. The AAV vector may be purified, replication incompetent, pseudotyped rAAV particles.

AAV are not associated with any known human diseases, are generally not considered pathogenic, and do not appear to alter the physiological properties of the host cell upon integration. AAV can infect a wide range of host cells, including non-dividing cells, and can infect cells from different species. In contrast to some vectors, which are quickly cleared or inactivated by both cellular and humoral responses, AAV vectors have been shown to induce persistent transgene expression in various tissues in vivo. The persistence of recombinant AAV-mediated transgenes in non-diving cells in vivo may be attributed to the lack of native AAV viral genes and the vector's ITR-linked ability to form episomal concatemers.

AAV is an attractive vector system for use in the cell transduction of the present invention as it has a high frequency of persistence as an episomal concatemer and it can infect non-dividing cells, including cardiomyocytes, thus making it useful for delivery of genes into mammalian cells, for example, in tissue culture and in vivo.

Typically, rAAV is made by cotransfecting a plasmid containing the gene of interest flanked by the two AAV terminal repeats and/or an expression plasmid containing the wild-type AAV coding sequences without the terminal repeats, for example pIM45. The cells are also infected and/or transfected with adenovirus and/or plasmids carrying the adenovirus genes required for AAV helper function. Stocks of rAAV made in such fashion are contaminated with adenovirus, which must be physically separated from the rAAV particles (for example, by cesium chloride density centrifugation or column chromatography). Alternatively, adenovirus vectors containing the AAV coding regions and/or cell lines containing the AAV coding regions and/or some or all of the adenovirus helper genes could be used. Cell lines carrying the rAAV DNA as an integrated provirus can also be used.

Multiple serotypes of AAV exist in nature, with at least twelve serotypes (AAV1-AAV12). Despite the high degree of homology, the different serotypes have tropisms for different tissues. Upon transfection, AAV elicits only a minor immune reaction (if any) in the host. Therefore, AAV is highly suited for gene therapy approaches.

The present disclosure may be directed in some embodiments to a drug comprising an AAV vector that is one or more of AAV1, AAV2, AAV3, AAV4, AAV5, AAV6, AAV7, AAV8, AAV9, AAV10, AAV11, AAV12, ANC AAV, chimeric AAV derived thereof, variations thereof, and combinations thereof, which will be even better suitable for high efficiency transduction in the tissue of interest. In certain embodiments, the gene therapy vector is an AAV serotype 1 vector. In certain embodiments, the gene therapy vector is an AAV serotype 2 vector. In certain embodiments, the gene therapy vector is an AAV serotype 3 vector. In certain embodiments, the gene therapy vector is an AAV serotype 4 vector. In certain embodiments, the gene therapy vector is an AAV serotype 5 vector. In certain embodiments, the gene therapy vector is an AAV serotype 6 vector. In certain embodiments, the gene therapy vector is an AAV serotype 7 vector. In certain embodiments, the gene therapy vector is an AAV serotype 8 vector. In certain embodiments, the gene therapy vector is an AAV serotype 9 vector. In certain embodiments, the gene therapy vector is an AAV serotype 10 vector. In certain embodiments, the gene therapy vector is an AAV serotype 11 vector. In certain embodiments, the gene therapy vector is an AAV serotype 12 vector.

In some embodiments, the gene therapy vector may be an AAV serotype having one or more capsid proteins disclosed in U.S. Pat. Nos. 7,198,951 and 7,906,111, the disclosures of which are hereby incorporated by reference herein in their entireties.

In some embodiments, the gene therapy vector is an AAV serotype 9 vector. One or more capsid proteins of the AAV serotype 9 vector may be selected from amino acid sequences of at least one of SEQ ID NO: 2, SEQ ID NO: 3, or portions thereof (e.g., amino acids 138 to 736 or amino acids 203 to 736 of either of SEQ ID NO: 2 or SEQ ID NO: 3).

One or more of the capsid proteins may be encoded by, for example, the nucleic acid sequences of SEQ ID NO: 4, SEQ ID NO: 5, or portions thereof (such as nucleotides 411 to 2211 or nucleotides 609 to 2211 of SEQ ID NO: 5).

A suitable dose of AAV for humans may be in the range of about 1×10⁸ vg/kg to about 3×10¹⁴ vg/kg, about 1×10⁸ vg/kg, about 1×10⁹ vg/kg, about 1×10¹⁰ vg/kg, about 1×10¹¹ vg/kg, about 1×10¹² vg/kg, about 1×10¹³ vg/kg, or about 1×10¹⁴ vg/kg. The total amount of viral particles or DRP is, is about, is at least, is at least about, is not more than, or is not more than about, 5×10¹⁵ vg/kg, 4×10¹⁵ vg/kg, 3×10¹⁵ vg/kg, 2×10¹⁵ vg/kg, 1×10¹⁵ vg/kg, 9×10¹⁴ vg/kg, 8×10¹⁴ vg/kg, 7×10¹⁴ vg/kg, 6×10¹⁴ vg/kg, 5×10¹⁴ vg/kg, 4×10¹⁴ vg/kg, 3×10¹⁴ vg/kg, 2×10¹⁴ vg/kg, 1×10¹⁴ vg/kg, 9×10¹³ vg/kg, 8×10¹³ vg/kg, 7×10¹³ vg/kg, 6×10¹³ vg/kg, 5×10¹³ vg/kg, 4×10¹³ vg/kg, 3×10¹³ vg/kg, 2×10¹³ vg/kg, 1×10¹³ vg/kg, 9×10¹² vg/kg, 8×10¹² vg/kg, 7×10¹² vg/kg, 6×10¹² vg/kg, 5×10¹² vg/kg, 4×10¹² vg/kg, 3×10¹² vg/kg, 2×10¹² vg/kg, 1×10¹² vg/kg, 9×10¹¹ vg/kg, 8×10¹¹ vg/kg, 7×10¹¹ vg/kg, 6×10¹¹ vg/kg, 5×10¹¹ vg/kg, 4×10¹¹ vg/kg, 3×10¹¹ vg/kg, 2×10¹¹ vg/kg, 1×10¹¹ vg/kg, 9×10¹⁰ vg/kg, 8×10¹⁰ vg/kg, 7×10¹⁰ vg/kg, 6×10¹⁰ vg/kg, 5×10¹⁰ vg/kg, 4×10¹⁰ vg/kg, 3×10¹⁰ vg/kg, 2×10¹⁰ vg/kg, 1×10¹⁰ vg/kg, 9×10⁹ vg/kg, 8×10⁹ vg/kg, 7×10⁹ vg/kg, 6×10⁹ vg/kg, 5×10⁹ vg/kg, 4×10⁹ vg/kg, 3×10⁹ vg/kg, 2×10⁹ vg/kg, 1×10⁹ vg/kg, 9×10⁸ vg/kg, 8×10⁸ vg/kg, 7×10⁸ vg/kg, 6×10⁸ vg/kg, 5×10⁸ vg/kg, 4×10⁸ vg/kg, 3×10⁸ vg/kg, 2×10⁸ vg/kg, or 1×10⁸ vg/kg, or falls within a range defined by any two of these values. The above listed dosages being in vg/kg heart tissue units.

Apart from viral vectors, non-viral expression constructs may also be used for introducing a gene encoding a target protein or a functioning variant or fragment thereof into a cell of a patient. Non-viral expression vectors which permit the in vivo expression of protein in the target cell include, for example, a plasmid, a modified RNA, a cDNA, antisense oligomers, DNA-lipid complexes, nanoparticles, exosomes, any other non-viral shuttle suitable for gene therapy, variations thereof, and a combination thereof.

Apart from viral vectors and non-viral expression vectors, nuclease systems may also be used, in conjunction with a vector and/or an electroporation system, to enter into a cell of a patient and introduce therein a gene encoding a target protein or a functioning variant or fragment thereof. Exemplary nuclease systems may include, without limitations, a clustered regularly interspaced short palindromic repeats (CRISPR), a DNA cutting enzyme (e.g., Cas9), meganucleases, TALENs, zinc finger nucleases, any other nuclease system suitable for gene therapy, variations thereof, and a combination thereof. For instance, in one embodiment, one viral vector (e.g., AAV) may be used for a nuclease (e.g., CRISPR) and another viral vector (e.g., AAV) may be used for a DNA cutting enzyme (e.g., Cas9) to introduce both (the nuclease and the DNA cutting enzyme) into a target cell.

Other vector delivery systems which can be employed to deliver a therapeutic polynucleotide sequence encoding a therapeutic gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific. Receptor-mediated gene targeting vehicles may include two components: a cell receptor-specific ligand and a DNA-binding agent.

Suitable methods for the transfer of non-viral vectors into target cells are, for example, the lipofection method, the calcium-phosphate co-precipitation method, the DEAE-dextran method and direct DNA introduction methods using micro-glass tubes, ultrasound, electroporation, and the like. Prior to the introduction of the vector, the cardiac muscle cells may be treated with a permeabilization agent, such as phosphatidylcholine, streptolysins, sodium caprate, decanoylcarnitine, tartaric acid, lysolecithin, Triton X-100, and the like. Exosomes may also be used to transfer naked DNA or AAV-encapsidated DNA.

A gene therapy vector of the invention may comprise a promoter that is functionally linked to the nucleic acid sequence encoding to the target protein. The promoter sequence must be compact and ensure a strong expression. Preferably, the promoter provides for an expression of the target protein in the myocardium of the patient that has been treated with the gene therapy vector. In some embodiment, the gene therapy vector comprises a cardiac-specific promoter that is operably linked to the nucleic acid sequence encoding the target protein. As used herein, a “cardiac-specific promoter” refers to a promoter whose activity in cardiac cells is at least 2-fold higher than in any other non-cardiac cell type. Preferably, a cardiac-specific promoter suitable for being used in the vector of the invention has an activity in cardiac cells which is at least 5-fold, at least 10-fold, at least 15-fold, at least 20-fold, at least 25-fold, or at least 50-fold higher compared to its activity in a non-cardiac cell type.

The cardiac-specific promoter may be a selected human promoter, or a promoter comprising a functionally equivalent sequence having at least about 80%, at least about 90%, at least about 95%, at least about 96%, at least about 97%, at least about 98%, or at least about 99% sequence identity to the selected human promoter. An exemplary non-limiting promoter that may be used is a cardiac troponin T promoter (TNNT2). Other non-limiting examples of promoters include the alpha myosin heavy chain promoter, the myosin light chain 2v promoter, the alpha myosin heavy chain promoter, the alpha-cardiac actin promoter, the alpha-tropomyosin promoter, the cardiac troponin C promoter, the cardiac troponin I promoter, the cardiac myosin-binding protein C promoter, and the sarco/endoplasmic reticulum Ca²⁺-ATPase (SERCA) promoter (e.g., isoform 2 of this promoter (SERCA2)).

The vectors useful in the present invention may have varying transduction efficiencies. As a result, the viral or non-viral vector transduces more than, equal to, or at least about 10%, about 20%, about 30%, about 40%, about 50%, about 55%, about 60%, about 65%, about 70%, about 75%, about 80%, about 85%, about 90%, about 95%, about 99%, or 100% of the cells of the targeted vascular territory. More than one vector (viral or non-viral, or combinations thereof) can be used simultaneously or in sequence. This can be used to transfer more than one polynucleotide, and/or target more than one type of cell. Where multiple vectors or multiple agents are used, more than one transduction/transfection efficiency can result.

Pharmaceutical compositions that contain gene therapy vectors may be prepared either as liquid solutions or suspensions. The pharmaceutical composition of the invention can include commonly used pharmaceutically acceptable excipients, such as diluents and carriers. In particular, the composition comprises a pharmaceutically acceptable carrier, e.g., water, saline, Ringer's solution, or dextrose solution. In addition to the carrier, the pharmaceutical composition may also contain emulsifying agents, pH buffering agents, stabilizers, dyes and the like.

In certain embodiments, a pharmaceutical composition will comprise a therapeutically effective gene dose, which is a dose that is capable of preventing or treating cardiomyopathy in a subject, without being toxic to the subject. Prevention or treatment of cardiomyopathy may be assessed as a change in a phenotypic characteristic associated with cardiomyopathy with such change being effective to prevent or treat cardiomyopathy. Thus, a therapeutically effective gene dose is typically one that, when administered in a physiologically tolerable composition, is sufficient to improve or prevent the pathogenic heart phenotype in the treated subject.

In certain embodiments, gene therapy vectors may be transduced into a subject through several different methods, including intravenous delivery, intraarterial delivery, or intraperitoneal delivery. In some embodiments, a gene therapy vector may be administered directly to heart tissue, for example, by intracoronary administration. In some embodiments, tissue transduction of the myocardium may be achieved by catheter-mediated intramyocardial delivery, which may be used to transfer vector-free cDNA coupled to or uncoupled to transduction-enhancing carriers into myocardium.

In certain embodiments, the drug will comprise a therapeutically effective gene dose. A therapeutically effective gene dose is one that is capable of preventing or treating a particular heart condition in a patient, without being toxic to the patient.

Heart conditions that may be treated by the methods disclosed herein may include, without limitations, one or more of a genetically determined heart disease (e.g., genetically determined cardiomyopathy), arrhythmic heart disease, heart failure, ischemia, arrhythmia, myocardial infarction, congestive heart failure, transplant rejection, abnormal heart contractility, non-ischemic cardiomyopathy, mitral valve regurgitation, aortic stenosis or regurgitation, abnormal Ca′ metabolism, congenital heart disease, primary or secondary cardiac tumors, and combinations thereof.

### ILLUSTRATIVE PROPHETIC EXAMPLES

The following example is set forth to assist in understanding the disclosure and should not, of course, be construed as specifically limiting the embodiments described and claimed herein. Such variations of the embodiments, including the substitution of all equivalents now known or later developed, which would be within the purview of those skilled in the art, and changes in formulation or minor changes in experimental design, are to be considered to fall within the scope of the embodiments incorporated herein.

In general, in vitro transduction efficiency may be assessed by qPCR, and quantification of concatamerization splicing events may be analyzed using specific primers and probes overlapping exonic junctions.

### Example 1: Homologous Overlapping Sequences

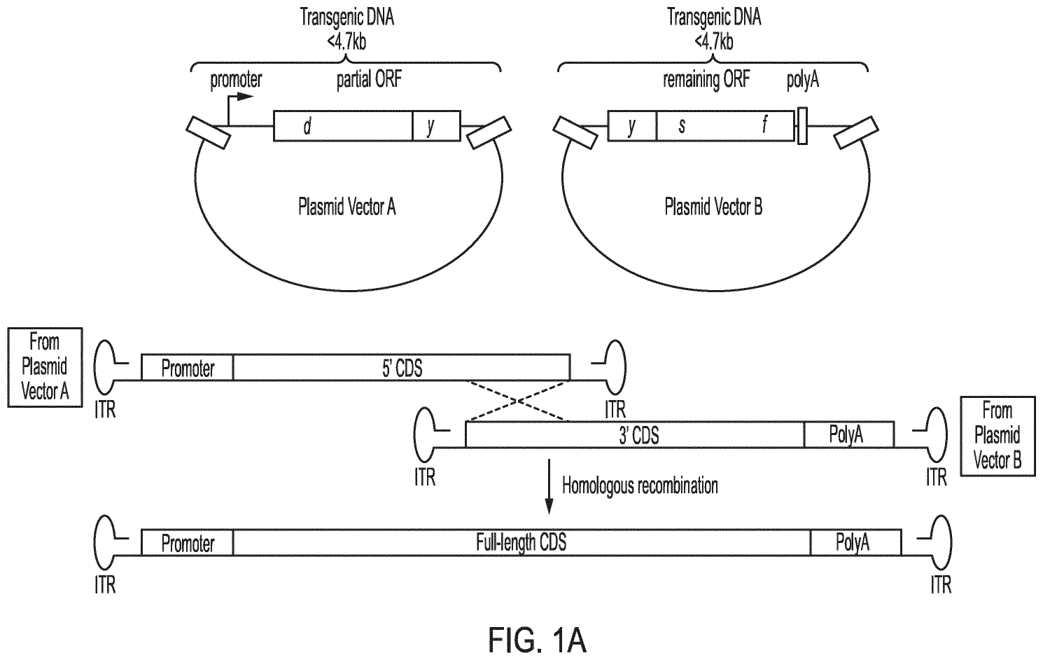

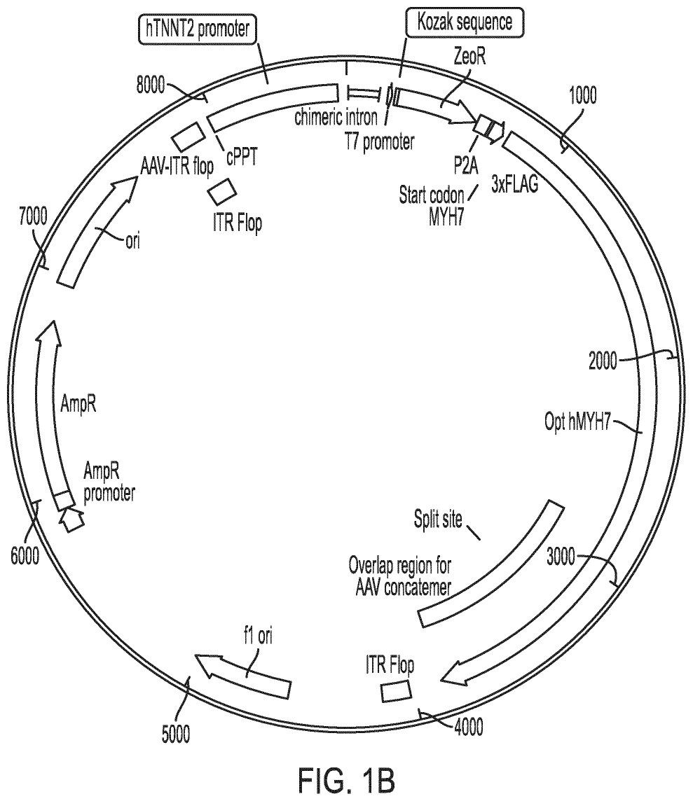

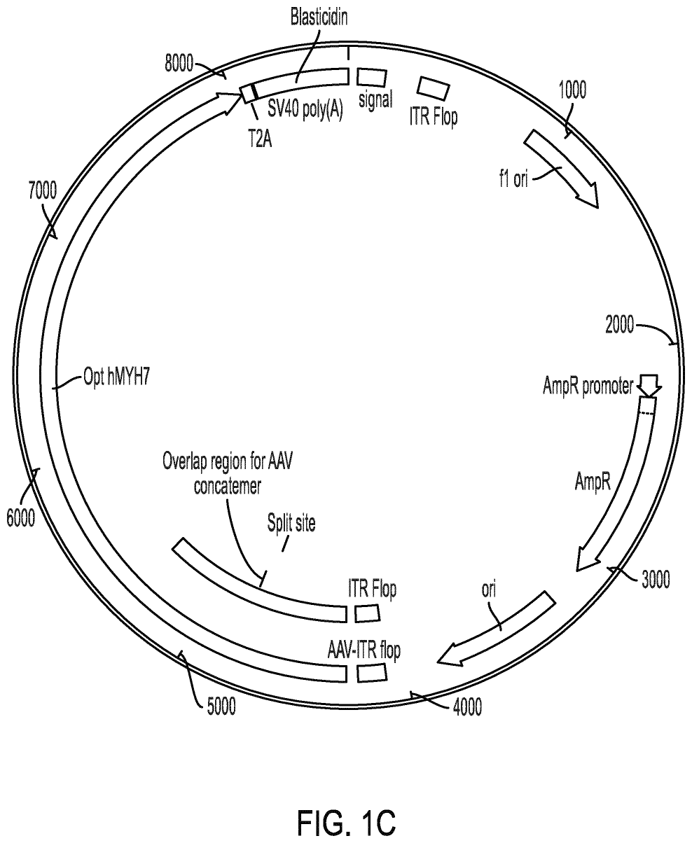

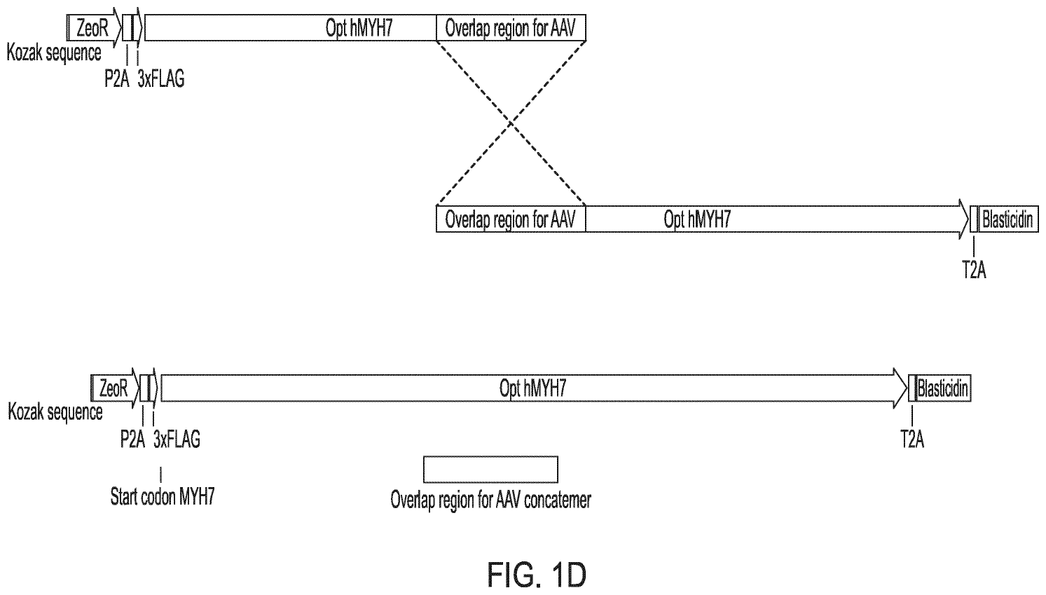

In this example, the transgene is split into two AAV vectors sharing homologous overlapping sequences, such that the reconstitution of MYH7 relies on homologous recombination. The overlap length can be adjusted, as discussed throughout this disclosure. In these example constructs, as illustrated in FIGS. 1A-1D, the size between the two ITR sequences is 4712 bases for the first AAV vector (FIG. 1B) and 4351 bases for the second AAV vector (FIG. 1C). The length of the overlap is 1055 bases (FIG. 1D).

SEQ ID NO: 6 corresponds to the first AAV vector of FIG. 1B having a Zeomycin resistance (ZeoR) gene removed, and SEQ ID NO: 7 corresponds to the second AAV vector of FIG. 1C.

### Example 2: Inteins

In this example, protein splicing occurs based on encoded intein sequences. The splicing event is an autocatalytic process where the intein excises itself from the primary/precursor protein and then catalyzes the joining of the broken ends forming two protein products: the mature protein and the intein itself.

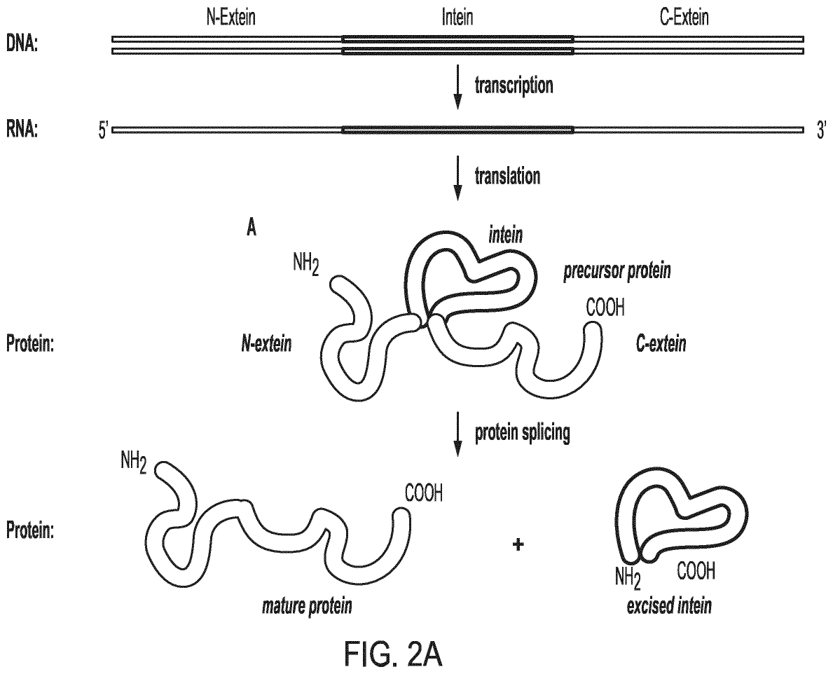

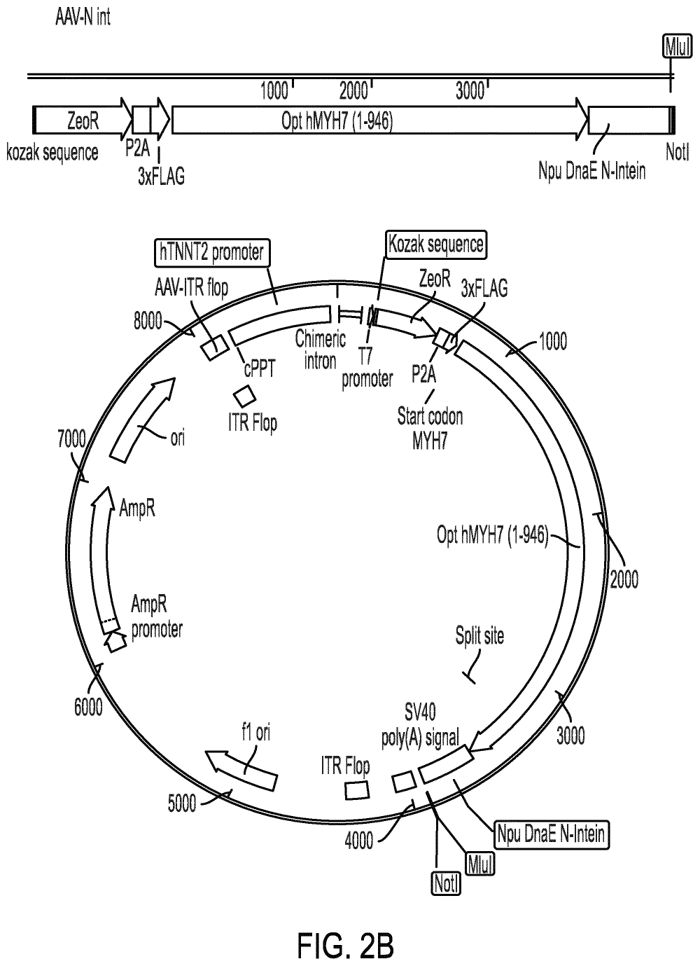

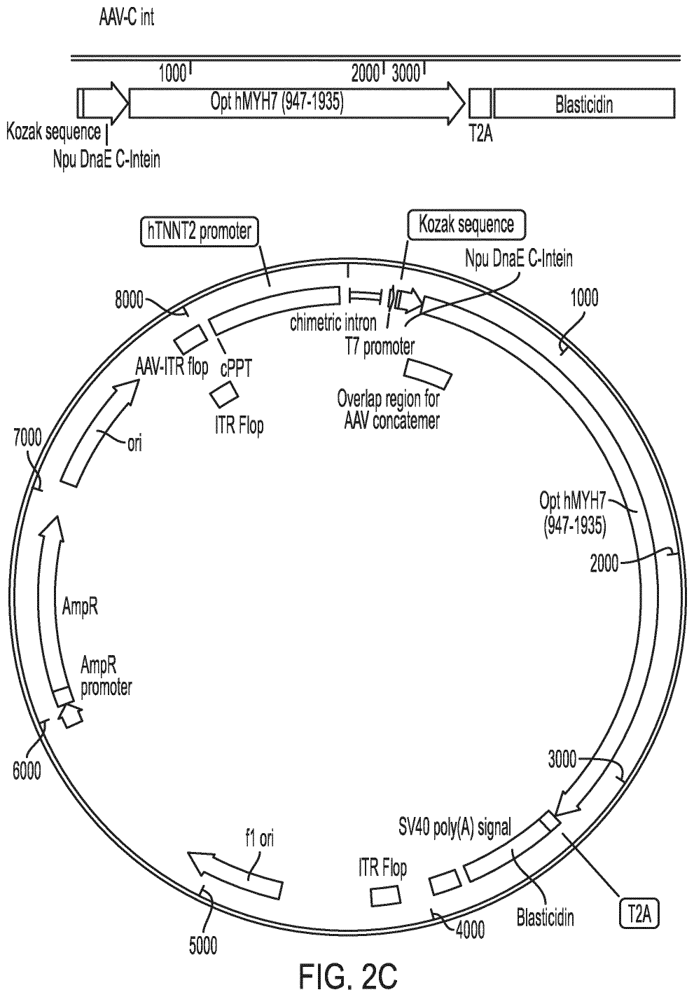

In these example constructs, as illustrated in FIGS. 2A-2C, the first AAV vector encodes the first N-terminal 946 amino acids (FIG. 2B, not to scale) and the second AAV vector encodes the C-terminal amino acids 947-1935 (FIG. 2C, not to scale), though it is contemplated that other combinations of sequence ranges are possible. The size between the two ITR sequences is 4923 bases for the first AAV vector (FIG. 2B, SEQ ID NO: 8) and 4819 bases for the second AAV vector (FIG. 2C, SEQ ID NO: 9). MYH7 expression is under the control of the TNNT2 promoter. Flag, ZeoR, and Blasticidin are used to select the transduced cells.

### Example 3: Hybrid Homologous Recombination and RNA Splicing

This example combines two approaches: homologous recombination and RNA splicing. A highly recombinogenic exogenous sequence is used to trigger the homologous recombination. This sequence is spliced out after transcription because it will be recognized as an intron in the pre-mRNA. This sequence was placed between exons 20 and 21, though other possible insertion locations may exist and are contemplated. This sequence is derived from the alkaline phosphatase gene (SEAP).

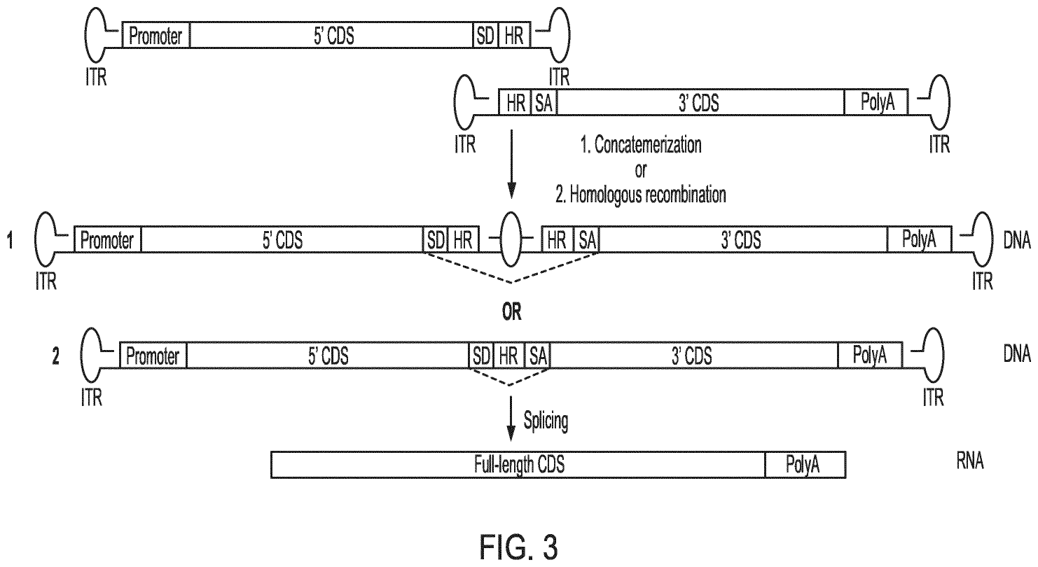

In these example constructs, as illustrated in FIG. 3, the first AAV vector (SEQ ID NO: 10) contains the TNNT2 promoter driving the transcription of the first 20 exons of MYH7. An optimized sequenced is generally used to improve protein translation in the other examples, but in this example sequences of exons 20 and 21 are not optimized. Exon 20 is followed by the first 40 bases of endogenous intron 20, followed by the first 272 bases of the SEAP gene. The selection gene, chimeric intron, and flag are also present to improve transcription/translation efficacy and imaging.

The second AAV vector (SEQ ID NO: 11) starts with the same 272 bases from the SEAP (for HR), followed by the last 40 bases of endogenous intron 20 and the whole non-optimized exon 21 of MYH7, followed by the sequence coding optimized exons 22-40 of MYH7.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is simply intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

SEQ ID NO: 1 below is an amino acid sequence encoding for MYH7:

MGDSEMAVFGAAAPYLRKSEKERLEAQTRPFDLKKDVFVPDDKQEFVKAK
IVSREGGKVTAETEYGKTVTVKEDQVMQQNPPKFDKIEDMAMLTFLHEPA
VLYNLKDRYGSWMIYTYSGLFCVTVNPYKWLPVYTPEVVAAYRGKKRSEA
PPHIFSISDNAYQYMLTDRENQSILITGESGAGKTVNTKRVIQYFAVIAA
IGDRSKKDQSPGKGTLEDQIIQANPALEAFGNAKTVRNDNSSRFGKFIRI
HFGATGKLASADIETYLLEKSRVIFQLKAERDYHIFYQILSNKKPELLDM
LLITNNPYDYAFISQGETTVASIDDAEELMATDNAFDVLGFTSEEKNSMY
KLTGAIMHFGNMKFKLKQREEQAEPDGTEEADKSAYLMGLNSADLLKGLC
HPRVKVGNEYVTKGQNVQQVIYATGALAKAVYERMFNWMVTRINATLETK
QPRQYFIGVLDIAGFEIFDFNSFEQLCINFTNEKLQQFFNHHMFVLEQEE
YKKEGIEWTFIDFGMDLQACIDLIEKPMGIMSILEEECMFPKATDMTFKA
KLFDNHLGKSANFQKPRNIKGKPEAHFSLIHYAGIVDYNIIGWLQKNKDP
LNETVVGLYQKSSLKLLSTLFANYAGADAPIEKGKGKAKKGSSFQTVSAL
HRENLNKLMTNLRSTHPHFVRCIIPNETKSPGVMDNPLVMHQLRCNGVLE
GIRICRKGFPNRILYGDFRQRYRILNPAAIPEGQFIDSRKGAEKLLSSLD
IDHNQYKFGHTKVFFKAGLLGLLEEMRDERLSRIITRIQAQSRGVLARME
YKKLLERRDSLLVIQWNIRAFMGVKNWPWMKLYFKIKPLLKSAEREKEMA
SMKEEFTRLKEALEKSEARRKELEEKMVSLLQEKNDLQLQVQAEQDNLAD
AEERCDQLIKNKIQLEAKVKEMNERLEDEEEMNAELTAKKRKLEDECSEL
KRDIDDLELTLAKVEKEKHATENKVKNLTEEMAGLDEIIAKLTKEKKALQ
EAHQQALDDLQAEEDKVNTLTKAKVKLEQQVDDLEGSLEQEKKVRMDLER
AKRKLEGDLKLTQESIMDLENDKQQLDERLKKKDFELNALNARIEDEQAL
GSQLQKKLKELQARIEELEEELEAERTARAKVEKLRSDLSRELEEISERL
EEAGGATSVQIEMNKKREAEFQKMRRDLEEATLQHEATAAALRKKHADSV
AELGEQIDNLQRVKQKLEKEKSEFKLELDDVTSNMEQIIKAKANLEKMCR
TLEDQMNEHRSKAEETQRSVNDLTSQRAKLQTENGELSRQLDEKEALISQ
LTRGKLTYTQQLEDLKRQLEEEVKAKNALAHALQSARHDCDLLREQYEEE
TEAKAELQRVLSKANSEVAQWRTKYETDAIQRTEELEEAKKKLAQRLQEA
EEAVEAVNAKCSSLEKTKHRLQNEIEDLMVDVERSNAAAAALDKKQRNFD
KILAEWKQKYEESQSELESSQKEARSLSTELFKLKNAYEESLEHLETFKR
ENKNLQEEISDLTEQLGSSGKTIHELEKVRKQLEAEKMELQSALEEAEAS
LEHEEGKILRAQLEFNQIKAEIERKLAEKDEEMEQAKRNHLRVVDSLQTS
LDAETRSRNEALRVKKKMEGDLNEMEIQLSHANRMAAEAQKQVKSLQSLL
KDTQIQLDDAVRANDDLKENIAIVERRNNLLQAELEELRAVVEQTERSRK
LAEQELIETSERVQLLHSQNTSLINQKKKMDADLSQLQTEVEEAVQECRN
AEEKAKKAITDAAMMAEELKKEQDTSAHLERMKKNMEQTIKDLQHRLDEA
EQIALKGGKKQLQKLEARVRELENELEAEQKRNAESVKGMRKSERRIKEL
TYQTEEDRKNLLRLQDLVDKLQLKVKAYKRQAEEAEEQANTNLSKFRKVQ
HELDEAEERADIAESQVNKLRAKSRDIGTKGLNEE

SEQ ID NO: 2 below is an amino acid sequence encoding for an AAV serotype 9 capsid protein.

MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPVEQSP
QEPDSSSGIGKSGQQPAKKRLNFGQTGDSESVPDPQPLGEPPEAPSGLGP
NTMASGGGAPMADNNEGADGVGNSSGNWHCDSTWLGDRVITTSTRTWALP
TYNNHLYKQISNGTSGGSTNDNTYFGYSTPWGYFDFNRFHCHFSPRDWQR
LINNNWGFRPKRLNFKLFNIQVKEVTTNEGTKTIANNLTSTVQVFTDSEY
QLPYVLGSAHQGCLPPFPADVFMVPQYGYLTLNNGSQALGRSSFYCLEYF
PSQMLRTGNNFQFSYTFEDVPFHSSYAHSQSLDRLMNPLIDQYLYYLVRT
QTTGTGGTQTLAFSQAGPSSMANQARNWVPGPCYRQQRVSTTTNQNNNSN
FAWTGAAKFKLNGRDSLMNPGVAMASHKDDEDRFFPSSGVLIFGKQGAGN
DGVDYSQVLITDEEEIKATNPVATEEYGAVAINNQAANTQAQTGLVHNQG
VIPGMVWQNRDVYLQGPIWAKIPHTDGNFHPSPLMGGFGLKHPPPQILIK
NTPVPADPPLTFNQAKLNSFITQYSTGQVSVEIEWELQKENSKRWNPEIQ
YTSNYYKSTNVDFAVNTEGVYSEPRPIGTRYLTRNL

SEQ ID NO: 3 below is a further amino acid sequence encoding for an AAV serotype 9 capsid protein:

MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIK
NTPVPADPPTAFNKDKLNSFITQYSTGQVSVEIEWELQKENSKRWNPEIQ
YTSNYYKSNNVEFAVNTEGVYSEPRPIGTRYLTRNL

SEQ ID NO: 4 below is a nucleic acid sequence encoding for an AAV serotype capsid protein:

ATGGCTGCCGATGGTTATCTTCCAGATTGGCTCGAGGACAACCTCTCTGA
GGGCATTCGCGAGTGGTGGGACCTGAAACCTGGAGCCCCGAAACCCAAAG
CCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGC
GGCGGACGCAGCGGCCCTCGAGCACGACAAGGCCTACGACCAGCAGCTCA
AAGCGGGTGACAATCCGTACCTGCGGTATAACCACGCCGACGCCGAGTTT
CAGGAGCGTCTGCAAGAAGATACGTCTTTTGGGGGCAACCTCGGGCGAGC
AGTCTTCCAGGCCAAGAAGCGGGTTCTCGAACCTCTCGGTCTGGTTGAGG
AAGGCGCTAAGACGGCTCCTGGAAAGAAGAGACCGGTAGAGCAGTCACCC
CAAGAACCAGACTCATCCTCGGGCATCGGCAAATCAGGCCAGCAGCCCGC
TAAAAAGAGACTCAATTTTGGTCAGACTGGCGACTCAGAGTCAGTCCCCG
ACCCACAACCTCTCGGAGAACCTCCAGAAGCCCCCTCAGGTCTGGGACCT
AATACAATGGCTTCAGGCGGTGGCGCTCCAATGGCAGACAATAACGAAGG
CGCCGACGGAGTGGGTAATTCCTCGGGAAATTGGCATTGCGATTCCACAT
GGCTGGGGGACAGAGTCATCACCACCAGCACCCGAACCTGGGCATTGCCC
ACCTACAACAACCACCTCTACAAGCAAATCTCCAATGGAACATCGGGAGG
AAGCACCAACGACAACACCTACTTTGGCTACAGCACCCCCTGGGGGTATT
TTGACTTCAACAGATTCCACTGCCACTTCTCACCACGTGACTGGCAGCGA
CTCATCAACAACAACTGGGGATTCCGGCCAAAGAGACTCAACTTCAAGCT
GTTCAACATCCAGGTCAAGGAGGTTACGACGAACGAAGGCACCAAGACCA
TCGCCAATAACCTTACCAGCACCGTCCAGGTCTTTACGGACTCGGAGTAC
CAGCTACCGTACGTCCTAGGCTCTGCCCACCAAGGATGCCTGCCACCGTT
TCCTGCAGACGTCTTCATGGTTCCTCAGTACGGCTACCTGACGCTCAACA
ATGGAAGTCAAGCGTTAGGACGTTCTTCTTTCTACTGTCTGGAATACTTC
CCTTCTCAGATGCTGAGAACCGGCAACAACTTTCAGTTCAGCTACACTTT
CGAGGACGTGCCTTTCCACAGCAGCTACGCACACAGCCAGAGTCTAGATC
GACTGATGAACCCCCTCATCGACCAGTACCTATACTACCTGGTCAGAACA
CAGACAACTGGAACTGGGGGAACTCAAACTTTGGCATTCAGCCAAGCAGG
CCCTAGCTCAATGGCCAATCAGGCTAGAAACTGGGTACCCGGGCCTTGCT
ACCGTCAGCAGCGCGTCTCCACAACCACCAACCAAAATAACAACAGCAAC
TTTGCGTGGACGGGAGCTGCTAAATTCAAGCTGAACGGGAGAGACTCGCT
AATGAATCCTGGCGTGGCTATGGCATCGCACAAAGACGACGAGGACCGCT
TCTTTCCATCAAGTGGCGTTCTCATATTTGGCAAGCAAGGAGCCGGGAAC
GATGGAGTCGACTACAGCCAGGTGCTGATTACAGATGAGGAAGAAATTAA
AGCCACCAACCCTGTAGCCACAGAGGAATACGGAGCAGTGGCCATCAACA
ACCAGGCCGCTAACACGCAGGCGCAAACTGGACTTGTGCATAACCAGGGA
GTTATTCCTGGTATGGTCTGGCAGAACCGGGACGTGTACCTGCAGGGCCC
TATTTGGGCTAAAATACCTCACACAGATGGCAACTTTCACCCGTCTCCTC
TGATGGGTGGATTTGGACTGAAACACCCACCTCCACAGATTCTAATTAAA
AATACACCAGTGCCGGCAGATCCTCCTCTTACCTTCAATCAAGCCAAGCT
GAACTCTTTCATCACGCAGTACAGCACGGGACAAGTCAGCGTGGAAATCG
AGTGGGAGCTGCAGAAAGAAAACAGCAAGCGCTGGAATCCAGAGATCCAG
TATACTTCAAACTACTACAAATCTACAAATGTGGACTTTGCTGTCAATAC
CGAAGGTGTTTACTCTGAGCCTCGCCCCATTGGTACTCGTTACCTCACCC
GTAATTTG

SEQ ID NO: 5 below is a further nucleic acid sequence encoding for an AAV serotype capsid protein:

ATGGCTGCCGATGGTTATCTTCCAGATTGGCTCGAGGACAACCTTAGTGA
AGGAATTCGCGAGTGGTGGGCTTTGAAACCTGGAGCCCCTCAACCCAAGG
CAAATCAACAACATCAAGACAACGCTCGAGGTCTTGTGCTTCCGGGTTAC
AAATACCTTGGACCCGGCAACGGACTCGACAAGGGGGAGCCGGTCAACGC
AGCAGACGCGGCGGCCCTCGAGCACGACAAGGCCTACGACCAGCAGCTCA
AGGCCGGAGACAACCCGTACCTCAAGTACAACCACGCCGACGCCGAGTTC
CAGGAGCGGCTCAAAGAAGATACGTCTTTTGGGGGCAACCTCGGGCGAGC
AGTCTTCCAGGCCAAAAAGAGGCTTCTTGAACCTCTTGGTCTGGTTGAGG
AAGCGGCTAAGACGGCTCCTGGAAAGAAGAGGCCTGTAGAGCAGTCTCCT
CAGGAACCGGACTCCTCCGCGGGTATTGGCAAATCGGGTGCACAGCCCGC
TAAAAAGAGACTCAATTTCGGTCAGACTGGCGACACAGAGTCAGTCCCAG
ACCCTCAACCAATCGGAGAACCTCCCGCAGCCCCCTCAGGTGTGGGATCT
CTTACAATGGCTTCAGGTGGTGGCGCACCAGTGGCAGACAATAACGAAGG
TGCCGATGGAGTGGGTAGTTCCTCGGGAAATTGGCATTGCGATTCCCAAT
GGCTGGGGGACAGAGTCATCACCACCAGCACCCGAACCTGGGCCCTGCCC
ACCTACAACAATCACCTCTACAAGCAAATCTCCAACAGCACATCTGGAGG
ATCTTCAAATGACAACGCCTACTTCGGCTACAGCACCCCCTGGGGGTATT
TTGACTTCAACAGATTCCACTGCCACTTCTCACCACGTGACTGGCAGCGA
CTCATCAACAACAACTGGGGATTCCGGCCTAAGCGACTCAACTTCAAGCT
CTTCAACATTCAGGTCAAAGAGGTTACGGACAACAATGGAGTCAAGACCA
TCGCCAATAACCTTACCAGCACGGTCCAGGTCTTCACGGACTCAGACTAT
CAGCTCCCGTACGTGCTCGGGTCGGCTCACGAGGGCTGCCTCCCGCCGTT
CCCAGCGGACGTTTTCATGATTCCTCAGTACGGGTATCTGACGCTTAATG
ATGGAAGCCAGGCCGTGGGTCGTTCGTCCTTTTACTGCCTGGAATATTTC
CCGTCGCAAATGCTAAGAACGGGTAACAACTTCCAGTTCAGCTACGAGTT
TGAGAACGTACCTTTCCATAGCAGCTACGCTCACAGCCAAAGCCTGGACC
GACTAATGAATCCACTCATCGACCAATACTTGTACTATCTCTCAAAGACT
ATTAACGGTTCTGGACAGAATCAACAAACGCTAAAATTCAGTGTGGCCGG
ACCCAGCAACATGGCTGTCCAGGGAAGAAACTACATACCTGGACCCAGCT
ACCGACAACAACGTGTCTCAACCACTGTGACTCAAAACAACAACAGCGAA
TTTGCTTGGCCTGGAGCTTCTTCTTGGGCTCTCAATGGACGTAATAGCTT
GATGAATCCTGGACCTGCTATGGCCAGCCACAAAGAAGGAGAGGACCGTT
TCTTTCCTTTGTCTGGATCTTTAATTTTTGGCAAACAAGGAACTGGAAGA
GACAACGTGGATGCGGACAAAGTCATGATAACCAACGAAGAAGAAATTAA
AACTACTAACCCGGTAGCAACGGAGTCCTATGGACAAGTGGCCACAAACC
ACCAGAGTGCCCAAGCACAGGCGCAGACCGGCTGGGTTCAAAACCAAGGA
ATACTTCCGGGTATGGTTTGGCAGGACAGAGATGTGTACCTGCAAGGACC
CATTTGGGCCAAAATTCCTCACACGGACGGCAACTTTCACCCTTCTCCGC
TGATGGGAGGGTTTGGAATGAAGCACCCGCCTCCTCAGATCCTCATCAAA
AACACACCTGTACCTGCGGATCCTCCAACGGCCTTCAACAAGGACAAGCT
GAACTCTTTCATCACCCAGTATTCTACTGGCCAAGTCAGCGTGGAGATCG
AGTGGGAGCTGCAGAAGGAAAACAGCAAGCGCTGGAACCCGGAGATCCAG
TACACTTCCAACTATTACAAGTCTAATAATGTTGAATTTGCTGTTAATAC
TGAAGGTGTATATAGTGAACCCCGCCCCATTGGCACCAGATACCTGACTC
GTAATCTGTAA

SEQ ID NO: 6 below is an AAV vector encoding a first portion of MYH7 that includes a homologous overlapping sequence:

GGCACTGGGCAGGTAAGTATCAAGGTTACAAGACAGGTTTAAGGAGACCAATAG
AAACTGGGCTTGTCGAGACAGAGAAGACTCTTGCGTTTCTGATAGGCACCTATTG
GTCTTACTGACATCCACTTTGCCTTTCTCTCCACAGGTGTCCACTCCCAGTTCAAT
TACAGCTCTTAAGGCTAGAGTACTTAATACGACTCACTATAGGCTAGCGGTACCG
GTCGCCACCATGGACTACAAAGACCATGACGGTGATTATAAAGATCATGACATC
GATTACAAGGATGACGATGACAAGCTTGGTACCGAGCTCGGATCCATGGGAGAT
TCTGAAATGGCCGTCTTCGGCGCTGCCGCACCATATCTCCGCAAAAGTGAAAAG
GAGCGCCTGGAGGCCCAAACCAGACCCTTCGATCTGAAAAAGGATGTGTTTGTC
CCGGATGACAAGCAAGAATTTGTAAAAGCAAAGATCGTCTCTCGGGAGGGCGGT
AAAGTGACTGCTGAAACAGAATACGGCAAAACCGTCACTGTCAAAGAAGATCAG
GTAATGCAACAAAATCCTCCGAAATTTGATAAAATCGAAGACATGGCCATGTTG
ACGTTTTTGCATGAGCCAGCTGTTTTGTATAATCTGAAGGATCGGTACGGATCCT
GGATGATTTACACATACTCAGGCTTGTTCTGCGTGACCGTTAATCCCTATAAGTG
GCTGCCTGTCTACACGCCGGAGGTTGTTGCAGCATATAGGGGGAAAAAGAGGTC
CGAAGCCCCTCCCCACATCTTCTCCATTTCTGACAACGCCTACCAGTATATGCTG
ACTGACAGGGAGAACCAAAGCATTCTTATTACCGGAGAGTCTGGGGCGGGCAAG
ACGGTAAATACAAAACGCGTGATACAGTACTTTGCAGTTATTGCTGCGATTGGCG
ACAGGAGTAAAAAGGATCAGAGCCCCGGCAAGGGTACTCTCGAAGATCAAATTA
TCCAGGCTAATCCCGCCCTCGAAGCGTTCGGGAATGCCAAAACGGTCCGCAATG
ATAACAGTAGCAGATTTGGGAAGTTTATCCGCATCCATTTCGGAGCGACTGGCAA
ACTCGCCTCAGCAGACATCGAGACATACCTGTTGGAAAAGTCACGAGTGATATTT
CAGCTCAAAGCCGAGCGGGACTATCATATCTTTTACCAGATACTTTCTAACAAAA
AACCAGAGCTTCTTGACATGCTTCTGATCACTAACAATCCGTATGACTATGCATT
CATCAGTCAGGGAGAGACCACGGTTGCGTCAATAGACGATGCTGAGGAGCTGAT
GGCCACAGACAATGCCTTCGATGTGCTCGGATTTACTAGCGAGGAGAAGAACAG
CATGTACAAGCTTACCGGTGCTATCATGCACTTCGGCAATATGAAATTTAAACTC
AAGCAGCGGGAAGAACAGGCGGAGCCGGACGGAACTGAAGAGGCTGATAAAAG
TGCCTACCTCATGGGGCTTAACTCTGCCGACTTGCTTAAAGGATTGTGTCATCCA
CGGGTAAAGGTAGGTAATGAATATGTAACTAAGGGTCAAAATGTGCAACAAGTC
ATATACGCGACTGGGGCTCTGGCAAAAGCCGTTTACGAGAGAATGTTTAACTGG
ATGGTCACACGAATTAATGCCACTCTGGAGACAAAACAACCCCGGCAGTACTTT
ATAGGTGTGCTGGATATCGCAGGTTTCGAAATTTTCGATTTCAACTCTTTCGAGC
AACTTTGTATCAATTTCACCAACGAGAAGCTCCAACAATTTTTTAACCATCATAT
GTTCGTTCTCGAGCAAGAGGAATACAAAAAAGAGGGCATCGAATGGACTTTCAT
CGATTTCGGTATGGATCTTCAGGCTTGTATAGATCTCATCGAGAAACCGATGGGG
ATTATGAGTATTCTGGAGGAGGAATGTATGTTCCCGAAAGCCACTGACATGACAT
TTAAGGCCAAGCTGTTCGATAATCACTTGGGGAAGTCCGCCAATTTCCAGAAACC
TAGGAACATAAAAGGTAAGCCGGAGGCGCACTTCTCTCTTATCCATTACGCGGG
AATCGTCGATTATAACATTATTGGCTGGCTTCAAAAGAACAAGGATCCGCTTAAC
GAAACAGTGGTCGGCCTTTATCAGAAAAGCTCACTGAAACTTCTTAGTACGCTCT
TTGCTAATTACGCTGGTGCTGACGCTCCAATCGAGAAAGGCAAGGGAAAAGCTA
AGAAAGGCAGTAGCTTTCAAACAGTCTCTGCCCTGCACAGAGAGAATCTCAACA
AGCTCATGACCAATCTGCGGAGCACACATCCACATTTTGTCAGGTGCATAATCCC
TAATGAGACGAAAAGTCCAGGCGTAATGGACAATCCGCTGGTTATGCACCAGCT
CAGATGTAATGGAGTATTGGAAGGTATACGCATATGTAGAAAAGGCTTCCCAAA
CAGGATACTGTATGGTGACTTCCGACAACGCTATCGAATACTGAACCCCGCAGCC
ATTCCCGAGGGACAATTCATAGACAGCCGCAAGGGAGCGGAAAAGCTTCTTTCC
TCCCTCGACATAGACCATAACCAGTACAAATTTGGGCACACCAAAGTATTTTTCA
AAGCGGGCCTTCTTGGACTGTTGGAAGAGATGCGCGATGAAAGGCTGTCAAGGA
TAATAACCAGGATTCAAGCGCAATCTCGGGGAGTGCTCGCGAGGATGGAGTACA
AGAAATTGTTGGAACGGAGAGATTCCTTGCTTGTGATTCAGTGGAATATACGAGC
TTTCATGGGAGTAAAAAACTGGCCTTGGATGAAACTCTACTTTAAGATAAAGCCA
CTGCTGAAGTCCGCAGAGAGGGAGAAGGAGATGGCATCAATGAAGGAGGAATT
TACGCGCTTGAAAGAGGCTCTCGAGAAGTCCGAAGCTAGACGGAAGGAATTGGA
AGAGAAGATGGTATCTCTTTTGCAGGAGAAGAATGATCTTCAGTTGCAAGTACA
AGCGGAACAAGACAATCTCGCCGACGCAGAGGAAAGGTGTGATCAGTTGATCAA
AAATAAAATCCAACTGGAAGCCAAGGTGAAAGAAATGAATGAGCGCTTGGAGG
ACGAAGAAGAGATGAATGCAGAGTTGACTGCGAAGAAGCGGAAGCTCGAAGAC
GAATGCAGCGAGCTCAAGCGCGACATTGACGACCTGGAATTGACCCTTGCCAAA
GTGGAAAAGGAGAAACATGCAACCGAGAATAAGGTAAAGAATCTGACAGAAGA
GATGGCGGGACTTGACGAGATTATCGCGAAGCTTACAAAGGAGAAGAAGGCCCT
TCAGGAAGCGCACCAACAGGCCCTTGACGATCTTCAGGCCGAGGAAGACAAAGT
GAACACATTGACTAAAGCTAAAGTGAAGCTTGAGCAACAGGTCGATGACGAATT
GCATTCATTTTATGTTTCAGGTTCAGGGGGAGATGTGGGAGGTTTTTTAAAGCAA
GTAAAACCTCTACAAATGTGGTAAAATCCGATAAGGGACTAGAGCATGGCTACG
TAGATAAGTAGCATGGCGGGTTAATCATTAACTACAAGGAACCCCTAGTGATGG
AGTTGGCCACTCCCTCTCTGCGCGCTCGCTCGCTCACTGAGGCCGGGCGACCAAA
GGTCGCCCGACGCCCGGGCTTTGCCCGGGCGGCCTCAGTGAGCGAGCGAGCGCG
CCAGCTGGCGTAATAGCGAAGAGGCCCGCACCGATCGCCCTTCCCAACAGTTGC
GCAGCCTGAATGGCGAATGGAATTCCAGACGATTGAGCGTCAAAATGTAGGTAT
TTCCATGAGCGTTTTTCCGTTGCAATGGCTGGCGGTAATATTGTTCTGGATATTAC
CAGCAAGGCCGATAGTTTGAGTTCTTCTACTCAGGCAAGTGATGTTATTACTAAT
CAAAGAAGTATTGCGACAACGGTTAATTTGCGTGATGGACAGACTCTTTTACTCG
GTGGCCTCACTGATTATAAAAACACTTCTCAGGATTCTGGCGTACCGTTCCTGTC
TAAAATCCCTTTAATCGGCCTCCTGTTTAGCTCCCGCTCTGATTCTAACGAGGAA
AGCACGTTATACGTGCTCGTCAAAGCAACCATAGTACGCGCCCTGTAGCGGCGC
ATTAAGCGCGGCGGGTGTGGTGGTTACGCGCAGCGTGACCGCTACACTTGCCAG
CGCCCTAGCGCCCGCTCCTTTCGCTTTCTTCCCTTCCTTTCTCGCCACGTTCGCCG
GCTTTCCCCGTCAAGCTCTAAATCGGGGGCTCCCTTTAGGGTTCCGATTTAGTGCT
TTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAAT
AGTGGACTCTTGTTCCAAACTGGAACAACACTCAACCCTATCTCGGTCTATTCTTT
TGATTTATAAGGGATTTTGCCGATTTCGGCCTATTGGTTAAAAAATGAGCTGATT
TAACAAAAATTTAACGCGAATTTTAACAAAATATTAACGTCTACAATTTAAATAT
TTGCTTATACAATCTTCCTGTTTTTGGGGCTTTTCTGATTATCAACCGGGGTACAT
ATGATTGACATGCTAGTTTTACGATTACCGTTCATCGATTCTCTTGTTTGCTCCAG
ACTCTCAGGCAATGACCTGATAGCCTTTGTAGAGACCTCTCAAAAATAGCTACCC
TCTCCGGCATGAATTTATCAGCTAGAACGGTTGAATATCATATTGATGGTGATTT
GACTGTCTCCGGCCTTTCTCACCCGTTTGAATCTTTACCTACACATTACTCAGGCA
TTGCATTTAAAATATATGAGGGTTCTAAAAATTTTTATCCTTGCGTTGAAATAAA
GGCTTCTCCCGCAAAAGTATTACAGGGTCATAATGTTTTTGGTACAACCGATTTA
GCTTTATGCTCTGAGGCTTTATTGCTTAATTTTGCTAATTCTTTGCCTTGCCTGTAT
GATTTATTGGATGTTGGAATCGCCTGATGCGGTATTTTCTCCTTACGCATCTGTGC
GGTATTTCACACCGCATATGGTGCACTCTCAGTACAATCTGCTCTGATGCCGCAT
AGTTAAGCCAGCCCCGACACCCGCCAACACCCGCTGACGCGCCCTGACGGGCTT
GTCTGCTCCCGGCATCCGCTTACAGACAAGCTGTGACCGTCTCCGGGAGCTGCAT
GTGTCAGAGGTTTTCACCGTCATCACCGAAACGCGCGAGACGAAAGGGCCTCGT
GATACGCCTATTTTTATAGGTTAATGTCATGATAATAATGGTTTCTTAGACGTCAG
GTGGCACTTTTCGGGGAAATGTGCGCGGAACCCCTATTTGTTTATTTTTCTAAATA
CATTCAAATATGTATCCGCTCATGAGACAATAACCCTGATAAATGCTTCAATAAT
ATTGAAAAAGGAAGAGTATGAGTATTCAACATTTCCGTGTCGCCCTTATTCCCTT
TTTTGCGGCATTTTGCCTTCCTGTTTTTGCTCACCCAGAAACGCTGGTGAAAGTAA
AAGATGCTGAAGATCAGTTGGGTGCACGAGTGGGTTACATCGAACTGGATCTCA
ACAGCGGTAAGATCCTTGAGAGTTTTCGCCCCGAAGAACGTTTTCCAATGATGAG
CACTTTTAAAGTTCTGCTATGTGGCGCGGTATTATCCCGTATTGACGCCGGGCAA
GAGCAACTCGGTCGCCGCATACACTATTCTCAGAATGACTTGGTTGAGTACTCAC
CAGTCACAGAAAAGCATCTTACGGATGGCATGACAGTAAGAGAATTATGCAGTG
CTGCCATAACCATGAGTGATAACACTGCGGCCAACTTACTTCTGACAACGATCGG
AGGACCGAAGGAGCTAACCGCTTTTTTGCACAACATGGGGGATCATGTAACTCG
CCTTGATCGTTGGGAACCGGAGCTGAATGAAGCCATACCAAACGACGAGCGTGA
CACCACGATGCCTGTAGCAATGGCAACAACGTTGCGCAAACTATTAACTGGCGA
ACTACTTACTCTAGCTTCCCGGCAACAATTAATAGACTGGATGGAGGCGGATAA
AGTTGCAGGACCACTTCTGCGCTCGGCCCTTCCGGCTGGCTGGTTTATTGCTGAT
AAATCTGGAGCCGGTGAGCGTGGGTCTCGCGGTATCATTGCAGCACTGGGGCCA
GATGGTAAGCCCTCCCGTATCGTAGTTATCTACACGACGGGGAGTCAGGCAACT
ATGGATGAACGAAATAGACAGATCGCTGAGATAGGTGCCTCACTGATTAAGCAT
TGGTAACTGTCAGACCAAGTTTACTCATATATACTTTAGATTGATTTAAAACTTCA
TTTTTAATTTAAAAGGATCTAGGTGAAGATCCTTTTTGATAATCTCATGACCAAA
ATCCCTTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAAACAAAA
AAACCACCGCTACCAGCGGTGGTTTGTTTGCCGGATCAAGAGCTACCAACTCTTT
TTCCGAAGGTAACTGGCTTCAGCAGAGCGCAGATACCAAATACTGTCCTTCTAGT
GTAGCCGTAGTTAGGCCACCACTTCAAGAACTCTGTAGCACCGCCTACATACCTC
GCTCTGCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGCTGAA
CGGGGGGTTCGTGCACACAGCCCAGCTTGGAGCGAACGACCTACACCGAACTGA
GATACCTACAGCGTGAGCTATGAGAAAGCGCCACGCTTCCCGAAGGGAGAAAGG
CGGACAGGTATCCGGTAAGCGGCAGGGTCGGAACAGGAGAGCGCACGAGGGAG
CTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAA
ACGCCAGCAACGCGGCCTTTTTACGGTTCCTGGCCTTTTGCTGGCCTTTTGCTCAC
ATGTTCTTTCCTGCGTTATCCCCTGATTCTGTGGATAACCGTATTACCGCCTTTGA
GTGAGCTGATACCGCTCGCCGCAGCCGAACGACCGAGCGCAGCGAGTCAGTGAG
CGAGGAAGCGGAAGAGCGCCCAATACGCAAACCGCCTCTCCCCGCGCGTTGGCC
GATTCATTAATGCAGCTGCGCGCTCGCTCGCTCACTGAGGCCGCCCGGGCAAAGC
CCGGGCGTCGGGCGACCTTTGGTCGCCCGGCCTCAGTGAGCGAGCGAGCGCGCA
GAGAGGGAGTGGCCAACTCCATCACTAGGGGTTCCTTGTAGTTAATGATTAACCC
GCCATGCTACTTATCTACGTAGCCATGCTCTAGATGTCATGGAGAAGACCCACCT
TGCAGATGTCCTCACTGGGGCTGGCAGAGCCGGCAACCTGCCTAAGGCTGCTCA
GTCCATTAGGAGCCAGTAGCCTGGAAGATGTCTTTACCCCCAGCATCAGTTCAAG
TGGAGCAGCACATAACTCTTGCCCTCTGCCTTCCAAGATTCTGGTGCTGAGACTT
ATGGAGTGTCTTGGAGGTTGCCTTCTGCCCCCCAACCCTGCTCCCAGCTGGCCCT
CCCAGGCCTGGGTTGCTGGCCTCTGCTTTATCAGGATTCTCAAGAGGGACAGCTG
GTTTATGTTGCATGACTGTTCCCTGCATATCTGCTCTGGTTTTAAATAGCTTATCT
GAGCAGCTGGAGGACCACATGGGCTTATATGGCGTGGGGTACATGTTCCTGTAG
CCTTGTCCCTGGCACCTGCCAAAATAGCAGCCAACACCCCCCACCCCCACCGCCA
TCCCCCTGCCCCACCCGTCCCCTGTCGCACATTCCTCCCTCCGCAGGGCTGGCTCA
CCAGGCCCCAGCCCACATGCCTGCTTAAAGCCCTCTCCATCCTCTGCCTCACCCA
GTCCCCGCTGAGACTGAGCAGACGCCTCCAGAGCTCGGATCCTGAGAACTTCAG
GGTGAGTCTATGGGAC

SEQ ID NO: 7 below is an AAV vector encoding a second portion of MYH7 that includes a homologous overlapping sequence:

GGCCGCTTCCCTTTAGTGAGGGTTAATGCTTCGAGCAGAC
ATGATAAGATACATTGATGAGTTTGGACAAACCACAACTA
GAATGCAGTGAAAAAAATGCTTTATTTGTGAAATTTGTGA
TGCTATTGCTTTATTTGTAACCATTATAAGCTGCAATAAA
CAAGTTAACAACAACAATTGCATTCATTTTATGTTTCAGG
TTCAGGGGGAGATGTGGGAGGTTTTTTAAAGCAAGTAAAA
CCTCTACAAATGTGGTAAAATCCGATAAGGGACTAGAGCA
TGGCTACGTAGATAAGTAGCATGGCGGGTTAATCATTAAC
TACAAGGAACCCCTAGTGATGGAGTTGGCCACTCCCTCTC
TGCGCGCTCGCTCGCTCACTGAGGCCGGGCGACCAAAGGT
CGCCCGACGCCCGGGCTTTGCCCGGGCGGCCTCAGTGAGC
GAGCGAGCGCGCCAGCTGGCGTAATAGCGAAGAGGCCCGC
ACCGATCGCCCTTCCCAACAGTTGCGCAGCCTGAATGGCG
AATGGAATTCCAGACGATTGAGCGTCAAAATGTAGGTATT
TCCATGAGCGTTTTTCCGTTGCAATGGCTGGCGGTAATAT
TGTTCTGGATATTACCAGCAAGGCCGATAGTTTGAGTTCT
TCTACTCAGGCAAGTGATGTTATTACTAATCAAAGAAGTA
TTGCGACAACGGTTAATTTGCGTGATGGACAGACTCTTTT
ACTCGGTGGCCTCACTGATTATAAAAACACTTCTCAGGAT
TCTGGCGTACCGTTCCTGTCTAAAATCCCTTTAATCGGCC
TCCTGTTTAGCTCCCGCTCTGATTCTAACGAGGAAAGCAC
GTTATACGTGCTCGTCAAAGCAACCATAGTACGCGCCCTG
TAGCGGCGCATTAAGCGCGGCGGGTGTGGTGGTTACGCGC
AGCGTGACCGCTACACTTGCCAGCGCCCTAGCGCCCGCTC
CTTTCGCTTTCTTCCCTTCCTTTCTCGCCACGTTCGCCGG
CTTTCCCCGTCAAGCTCTAAATCGGGGGCTCCCTTTAGGG
TTCCGATTTAGTGCTTTACGGCACCTCGACCCCAAAAAAC
TTGATTAGGGTGATGGTTCACGTAGTGGGCCATCGCCCTG
ATAGACGGTTTTTCGCCCTTTGACGTTGGAGTCCACGTTC
TTTAATAGTGGACTCTTGTTCCAAACTGGAACAACACTCA
ACCCTATCTCGGTCTATTCTTTTGATTTATAAGGGATTTT
GCCGATTTCGGCCTATTGGTTAAAAAATGAGCTGATTTAA
CAAAAATTTAACGCGAATTTTAACAAAATATTAACGTCTA
CAATTTAAATATTTGCTTATACAATCTTCCTGTTTTTGGG
GCTTTTCTGATTATCAACCGGGGTACATATGATTGACATG
CTAGTTTTACGATTACCGTTCATCGATTCTCTTGTTTGCT
CCAGACTCTCAGGCAATGACCTGATAGCCTTTGTAGAGAC
CTCTCAAAAATAGCTACCCTCTCCGGCATGAATTTATCAG
CTAGAACGGTTGAATATCATATTGATGGTGATTTGACTGT
CTCCGGCCTTTCTCACCCGTTTGAATCTTTACCTACACAT
TACTCAGGCATTGCATTTAAAATATATGAGGGTTCTAAAA
ATTTTTATCCTTGCGTTGAAATAAAGGCTTCTCCCGCAAA
AGTATTACAGGGTCATAATGTTTTTGGTACAACCGATTTA
GCTTTATGCTCTGAGGCTTTATTGCTTAATTTTGCTAATT
CTTTGCCTTGCCTGTATGATTTATTGGATGTTGGAATCGC
CTGATGCGGTATTTTCTCCTTACGCATCTGTGCGGTATTT
CACACCGCATATGGTGCACTCTCAGTACAATCTGCTCTGA
TGCCGCATAGTTAAGCCAGCCCCGACACCCGCCAACACCC
GCTGACGCGCCCTGACGGGCTTGTCTGCTCCCGGCATCCG
CTTACAGACAAGCTGTGACCGTCTCCGGGAGCTGCATGTG
TCAGAGGTTTTCACCGTCATCACCGAAACGCGCGAGACGA
AAGGGCCTCGTGATACGCCTATTTTTATAGGTTAATGTCA
TGATAATAATGGTTTCTTAGACGTCAGGTGGCACTTTTCG
GGGAAATGTGCGCGGAACCCCTATTTGTTTATTTTTCTAA
ATACATTCAAATATGTATCCGCTCATGAGACAATAACCCT
GATAAATGCTTCAATAATATTGAAAAAGGAAGAGTATGAG
TATTCAACATTTCCGTGTCGCCCTTATTCCCTTTTTTGCG
GCATTTTGCCTTCCTGTTTTTGCTCACCCAGAAACGCTGG
TGAAAGTAAAAGATGCTGAAGATCAGTTGGGTGCACGAGT
GGGTTACATCGAACTGGATCTCAACAGCGGTAAGATCCTT
GAGAGTTTTCGCCCCGAAGAACGTTTTCCAATGATGAGCA
CTTTTAAAGTTCTGCTATGTGGCGCGGTATTATCCCGTAT
TGACGCCGGGCAAGAGCAACTCGGTCGCCGCATACACTAT
TCTCAGAATGACTTGGTTGAGTACTCACCAGTCACAGAAA
AGCATCTTACGGATGGCATGACAGTAAGAGAATTATGCAG
TGCTGCCATAACCATGAGTGATAACACTGCGGCCAACTTA
CTTCTGACAACGATCGGAGGACCGAAGGAGCTAACCGCTT
TTTTGCACAACATGGGGGATCATGTAACTCGCCTTGATCG
TTGGGAACCGGAGCTGAATGAAGCCATACCAAACGACGAG
CGTGACACCACGATGCCTGTAGCAATGGCAACAACGTTGC
GCAAACTATTAACTGGCGAACTACTTACTCTAGCTTCCCG
GCAACAATTAATAGACTGGATGGAGGCGGATAAAGTTGCA
GGACCACTTCTGCGCTCGGCCCTTCCGGCTGGCTGGTTTA
TTGCTGATAAATCTGGAGCCGGTGAGCGTGGGTCTCGCGG
TATCATTGCAGCACTGGGGCCAGATGGTAAGCCCTCCCGT
ATCGTAGTTATCTACACGACGGGGAGTCAGGCAACTATGG
ATGAACGAAATAGACAGATCGCTGAGATAGGTGCCTCACT
GATTAAGCATTGGTAACTGTCAGACCAAGTTTACTCATAT
ATACTTTAGATTGATTTAAAACTTCATTTTTAATTTAAAA
GGATCTAGGTGAAGATCCTTTTTGATAATCTCATGACCAA
AATCCCTTAACGTGAGTTTTCGTTCCACTGAGCGTCAGAC
CCCGTAGAAAAGATCAAAGGATCTTCTTGAGATCCTTTTT
TTCTGCGCGTAATCTGCTGCTTGCAAACAAAAAAACCACC
GCTACCAGCGGTGGTTTGTTTGCCGGATCAAGAGCTACCA
ACTCTTTTTCCGAAGGTAACTGGCTTCAGCAGAGCGCAGA
TACCAAATACTGTCCTTCTAGTGTAGCCGTAGTTAGGCCA
CCACTTCAAGAACTCTGTAGCACCGCCTACATACCTCGCT
CTGCTAATCCTGTTACCAGTGGCTGCTGCCAGTGGCGATA
AGTCGTGTCTTACCGGGTTGGACTCAAGACGATAGTTACC
GGATAAGGCGCAGCGGTCGGGCTGAACGGGGGGTTCGTGC
ACACAGCCCAGCTTGGAGCGAACGACCTACACCGAACTGA
GATACCTACAGCGTGAGCTATGAGAAAGCGCCACGCTTCC
CGAAGGGAGAAAGGCGGACAGGTATCCGGTAAGCGGCAGG
GTCGGAACAGGAGAGCGCACGAGGGAGCTTCCAGGGGGAA
ACGCCTGGTATCTTTATAGTCCTGTCGGGTTTCGCCACCT
CTGACTTGAGCGTCGATTTTTGTGATGCTCGTCAGGGGGG
CGGAGCCTATGGAAAAACGCCAGCAACGCGGCCTTTTTAC
GGTTCCTGGCCTTTTGCTGGCCTTTTGCTCACATGTTCTT
TCCTGCGTTATCCCCTGATTCTGTGGATAACCGTATTACC
GCCTTTGAGTGAGCTGATACCGCTCGCCGCAGCCGAACGA
CCGAGCGCAGCGAGTCAGTGAGCGAGGAAGCGGAAGAGCG
CCCAATACGCAAACCGCCTCTCCCCGCGCGTTGGCCGATT
CATTAATGCAGCTGCGCGCTCGCTCGCTCACTGAGGCCGC
CCGGGCAAAGCCCGGGCGTCGGGCGACCTTTGGTCGCCCG
GCCTCAGTGAGCGAGCGAGCGCGCAGAGAGGGAGTGGCCA
ACTCCATCACTAGGGGTTCCTTGTAGTTAATGATTAACCC
GCCATGCTACTTATCTACGTAGCCATGCTCTAGAGCGTAA
TGGACAATCCGCTGGTTATGCACCAGCTCAGATGTAATGG
AGTATTGGAAGGTATACGCATATGTAGAAAAGGCTTCCCA
AACAGGATACTGTATGGTGACTTCCGACAACGCTATCGAA
TACTGAACCCCGCAGCCATTCCCGAGGGACAATTCATAGA
CAGCCGCAAGGGAGCGGAAAAGCTTCTTTCCTCCCTCGAC
ATAGACCATAACCAGTACAAATTTGGGCACACCAAAGTAT
TTTTCAAAGCGGGCCTTCTTGGACTGTTGGAAGAGATGCG
CGATGAAAGGCTGTCAAGGATAATAACCAGGATTCAAGCG
CAATCTCGGGGAGTGCTCGCGAGGATGGAGTACAAGAAAT
TGTTGGAACGGAGAGATTCCTTGCTTGTGATTCAGTGGAA
TATACGAGCTTTCATGGGAGTAAAAAACTGGCCTTGGATG
AAACTCTACTTTAAGATAAAGCCACTGCTGAAGTCCGCAG
AGAGGGAGAAGGAGATGGCATCAATGAAGGAGGAATTTAC
GCGCTTGAAAGAGGCTCTCGAGAAGTCCGAAGCTAGACGG
AAGGAATTGGAAGAGAAGATGGTATCTCTTTTGCAGGAGA
AGAATGATCTTCAGTTGCAAGTACAAGCGGAACAAGACAA
TCTCGCCGACGCAGAGGAAAGGTGTGATCAGTTGATCAAA
AATAAAATCCAACTGGAAGCCAAGGTGAAAGAAATGAATG
AGCGCTTGGAGGACGAAGAAGAGATGAATGCAGAGTTGAC
TGCGAAGAAGCGGAAGCTCGAAGACGAATGCAGCGAGCTC
AAGCGCGACATTGACGACCTGGAATTGACCCTTGCCAAAG
TGGAAAAGGAGAAACATGCAACCGAGAATAAGGTAAAGAA
TCTGACAGAAGAGATGGCGGGACTTGACGAGATTATCGCG
AAGCTTACAAAGGAGAAGAAGGCCCTTCAGGAAGCGCACC
AACAGGCCCTTGACGATCTTCAGGCCGAGGAAGACAAAGT
GAACACATTGACTAAAGCTAAAGTGAAGCTTGAGCAACAG
GTCGATGACCTGGAAGGCTCACTTGAGCAAGAGAAGAAAG
TGCGCATGGATCTCGAGCGCGCTAAACGCAAGCTCGAGGG
AGATTTGAAGCTGACTCAAGAGTCAATTATGGACCTTGAA
AACGACAAGCAACAGTTGGATGAGAGACTTAAGAAGAAGG
ACTTCGAACTCAACGCACTTAATGCCAGGATCGAAGATGA
ACAAGCTCTCGGGTCTCAGCTCCAAAAGAAGCTTAAGGAA
CTCCAGGCACGCATAGAAGAACTCGAAGAAGAGTTGGAAG
CCGAGAGAACGGCGAGAGCTAAAGTGGAAAAACTCCGAAG
CGATTTGTCCAGAGAGCTGGAAGAGATTTCCGAGCGGCTT
GAGGAGGCGGGAGGCGCGACGAGCGTGCAGATCGAAATGA
ATAAGAAGAGAGAAGCTGAATTTCAAAAAATGCGCCGAGA
CCTTGAAGAAGCAACACTCCAGCACGAAGCCACAGCAGCT
GCGCTTCGGAAGAAGCATGCTGATTCTGTCGCAGAGCTGG
GGGAGCAGATCGACAATCTCCAGCGAGTCAAACAAAAACT
GGAAAAGGAGAAATCTGAGTTTAAATTGGAGCTTGACGAC
GTCACGTCTAACATGGAGCAGATAATAAAGGCAAAAGCAA
ACCTTGAGAAAATGTGCCGCACTCTTGAGGACCAGATGAA
CGAGCATCGCAGTAAGGCGGAAGAGACTCAGAGATCTGTT
AACGACTTGACCAGTCAACGAGCTAAACTTCAGACAGAAA
ATGGAGAACTCTCCCGACAACTGGACGAAAAGGAGGCCCT
GATCAGCCAGTTGACTAGGGGTAAACTCACCTACACACAG
CAGCTTGAGGACTTGAAGCGCCAACTCGAAGAGGAGGTGA
AAGCTAAAAACGCACTGGCACATGCGCTGCAATCCGCAAG
ACATGACTGCGACCTCCTGCGGGAACAGTACGAAGAAGAA
ACTGAGGCGAAAGCTGAGCTCCAACGGGTACTTTCTAAGG
CAAATTCTGAAGTTGCACAGTGGCGAACTAAGTACGAAAC
CGATGCGATCCAACGCACGGAGGAGTTGGAGGAAGCTAAA
AAGAAGCTCGCACAGCGACTGCAGGAAGCCGAAGAAGCCG
TTGAGGCAGTTAATGCAAAATGCAGTAGTCTCGAGAAGAC
TAAACATAGACTCCAGAACGAAATAGAGGATCTGATGGTG
GATGTTGAACGCTCCAATGCTGCTGCGGCAGCCCTCGACA
AAAAACAGCGGAATTTCGATAAGATTCTTGCGGAATGGAA
GCAGAAATACGAAGAGTCTCAGAGTGAACTGGAGAGCTCC
CAAAAGGAGGCCCGAAGTCTGTCTACCGAATTGTTTAAAC
TCAAGAACGCGTACGAAGAATCTCTGGAACACTTGGAAAC
CTTTAAAAGAGAGAACAAAAACCTGCAAGAGGAAATAAGT
GACCTTACCGAGCAACTGGGGAGCTCCGGGAAAACCATCC
ACGAATTGGAAAAAGTCAGGAAACAGTTGGAGGCAGAGAA
AATGGAGCTCCAATCCGCCCTGGAGGAAGCGGAAGCATCT
CTGGAACACGAGGAGGGCAAGATTCTGAGGGCCCAACTGG
AATTTAACCAGATCAAGGCAGAGATAGAACGAAAACTCGC
GGAGAAGGACGAAGAGATGGAGCAGGCGAAACGGAATCAT
CTGCGGGTTGTAGACTCCCTGCAAACCTCCCTCGATGCCG
AAACCAGAAGCCGGAATGAGGCCCTTCGGGTGAAAAAAAA
AATGGAGGGCGACTTGAACGAAATGGAAATTCAACTTTCT
CACGCCAACCGCATGGCGGCCGAGGCGCAGAAACAGGTAA
AATCTCTCCAGTCTCTCCTCAAAGATACACAAATCCAACT
CGACGATGCTGTAAGGGCAAACGATGATTTGAAAGAGAAT
ATAGCAATCGTCGAGCGCCGCAATAATCTGTTGCAAGCAG
AGCTTGAAGAACTGCGCGCGGTCGTAGAACAGACCGAACG
CAGTAGAAAGTTGGCTGAGCAGGAACTTATTGAGACTTCC
GAGCGCGTTCAGCTTCTGCATTCCCAGAACACCTCTCTGA
TTAATCAGAAGAAAAAGATGGACGCAGATCTGTCTCAGTT
GCAGACGGAAGTGGAAGAAGCTGTTCAAGAGTGCCGGAAC
GCGGAGGAAAAAGCTAAAAAGGCGATAACTGATGCAGCGA
TGATGGCTGAGGAACTGAAGAAGGAGCAAGACACCTCCGC
CCATTTGGAGCGAATGAAGAAGAATATGGAACAAACTATT
AAGGATCTCCAGCACCGGCTTGATGAGGCTGAACAGATCG
CCTTGAAAGGGGGGAAAAAGCAATTGCAGAAATTGGAAGC
CCGAGTAAGGGAACTGGAGAATGAGTTGGAAGCTGAGCAA
AAGCGGAACGCTGAGTCTGTGAAGGGAATGAGAAAGAGTG
AACGGAGGATAAAAGAACTCACATATCAAACGGAAGAGGA
CCGGAAAAACTTGCTCCGCCTCCAAGACCTCGTTGACAAG
CTTCAATTGAAAGTCAAGGCTTATAAAAGACAAGCTGAAG
AAGCTGAAGAACAAGCGAACACCAATTTGTCCAAATTTCG
GAAAGTACAGCATGAGCTCGACGAGGCTGAGGAGCGGGCT
GACATAGCTGAGTCCCAGGTCAATAAACTGCGAGCGAAAA
GCAGAGACATTGGCACCAAAGGTTTGAATGAAGAGACCGG
TGAGGGCAGAGGAAGTCTTCTAACATGCGGTGACGTGGAG
GAGAATCCCGGCCCTTCCGGTATGAAAACCTTCAACATCT
CTCAGCAGGATCTGGAGCTGGTGGAGGTCGCCACTGAGAA
GATCACCATGCTCTATGAGGACAACAAGCACCATGTCGGG
GCGGCCATCAGGACCAAGACTGGGGAGATCATCTCTGCTG
TCCACATTGAAGCCTACATTGGCAGGGTCACTGTCTGTGC
TGAAGCCATTGCCATTGGGTCTGCTGTGAGCAACGGGCAG
AAGGACTTTGACACCATTGTGGCTGTCAGGCACCCCTACT
CTGATGAGGTGGACAGATCCATCAGGGTGGTCAGCCCCTG
TGGCATGTGTAGAGAGCTGATCTCTGACTATGCTCCTGAC
TGCTTTGTGCTCATTGAGATGAATGGCAAGCTGGTCAAAA
CCACCATTGAGGAACTCATCCCCCTCAAGTACACCAGGAA
CTAATAAGC

SEQ ID NO: 8 below is an AAV vector encoding a first portion of MYH7 and an N-intein sequence:

GGCACTGGGCAGGTAAGTATCAAGGTTACAAGACAGGTTT
AAGGAGACCAATAGAAACTGGGCTTGTCGAGACAGAGAAG
ACTCTTGCGTTTCTGATAGGCACCTATTGGTCTTACTGAC
ATCCACTTTGCCTTTCTCTCCACAGGTGTCCACTCCCAGT
TCAATTACAGCTCTTAAGGCTAGAGTACTTAATACGACTC
ACTATAGGCTAGCGGTACCGGTCGCCACCATGATGGCCAA
ACTCACTTCTGCAGTCCCAGTCCTCACAGCAAGGGATGTT
GCAGGGGCTGTAGAGTTCTGGACTGACAGATTAGGATTCT
CCAGAGACTTTGTTGAAGATGATTTTGCTGGTGTTGTCAG
AGATGATGTCACCCTCTTCATCTCAGCAGTTCAGGACCAA
GTTGTCCCTGACAACACCCTTGCTTGGGTCTGGGTCAGAG
GCCTAGATGAGCTTTATGCAGAATGGTCAGAAGTAGTCAG
CACAAATTTCAGGGATGCCTCTGGCCCAGCCATGACAGAA
ATTGGTGAACAACCTTGGGGAAGGGAATTTGCCCTCAGAG
ACCCTGCTGGAAATTGTGTCCATTTTGTAGCTGAGGAACA
GGACGGATCTGGAGCAACAAACTTCTCACTACTCAAACAA
GCAGGTGACGTGGAGGAGAATCCCGGGCCTGACTACAAAG
ACCATGACGGTGATTATAAAGATCATGACATCGATTACAA
GGATGACGATGACAAGCTTGAGCTCGGATCCATGGGAGAT
TCTGAAATGGCCGTCTTCGGCGCTGCCGCACCATATCTCC
GCAAAAGTGAAAAGGAGCGCCTGGAGGCCCAAACCAGACC
CTTCGATCTGAAAAAGGATGTGTTTGTCCCGGATGACAAG
CAAGAATTTGTAAAAGCAAAGATCGTCTCTCGGGAGGGCG
GTAAAGTGACTGCTGAAACAGAATACGGCAAAACCGTCAC
TGTCAAAGAAGATCAGGTAATGCAACAAAATCCTCCGAAA
TTTGATAAAATCGAAGACATGGCCATGTTGACGTTTTTGC
ATGAGCCAGCTGTTTTGTATAATCTGAAGGATCGGTACGG
ATCCTGGATGATTTACACATACTCAGGCTTGTTCTGCGTG
ACCGTTAATCCCTATAAGTGGCTGCCTGTCTACACGCCGG
AGGTTGTTGCAGCATATAGGGGGAAAAAGAGGTCCGAAGC
CCCTCCCCACATCTTCTCCATTTCTGACAACGCCTACCAG
TATATGCTGACTGACAGGGAGAACCAAAGCATTCTTATTA
CCGGAGAGTCTGGGGCGGGCAAGACGGTAAATACAAAACG
CGTGATACAGTACTTTGCAGTTATTGCTGCGATTGGCGAC
AGGAGTAAAAAGGATCAGAGCCCCGGCAAGGGTACTCTCG
AAGATCAAATTATCCAGGCTAATCCCGCCCTCGAAGCGTT
CGGGAATGCCAAAACGGTCCGCAATGATAACAGTAGCAGA
TTTGGGAAGTTTATCCGCATCCATTTCGGAGCGACTGGCA
AACTCGCCTCAGCAGACATCGAGACATACCTGTTGGAAAA
GTCACGAGTGATATTTCAGCTCAAAGCCGAGCGGGACTAT
CATATCTTTTACCAGATACTTTCTAACAAAAAACCAGAGC
TTCTTGACATGCTTCTGATCACTAACAATCCGTATGACTA
TGCATTCATCAGTCAGGGAGAGACCACGGTTGCGTCAATA
GACGATGCTGAGGAGCTGATGGCCACAGACAATGCCTTCG
ATGTGCTCGGATTTACTAGCGAGGAGAAGAACAGCATGTA
CAAGCTTACCGGTGCTATCATGCACTTCGGCAATATGAAA
TTTAAACTCAAGCAGCGGGAAGAACAGGCGGAGCCGGACG
GAACTGAAGAGGCTGATAAAAGTGCCTACCTCATGGGGCT
TAACTCTGCCGACTTGCTTAAAGGATTGTGTCATCCACGG
GTAAAGGTAGGTAATGAATATGTAACTAAGGGTCAAAATG
TGCAACAAGTCATATACGCGACTGGGGCTCTGGCAAAAGC
CGTTTACGAGAGAATGTTTAACTGGATGGTCACACGAATT
AATGCCACTCTGGAGACAAAACAACCCCGGCAGTACTTTA
TAGGTGTGCTGGATATCGCAGGTTTCGAAATTTTCGATTT
CAACTCTTTCGAGCAACTTTGTATCAATTTCACCAACGAG
AAGCTCCAACAATTTTTTAACCATCATATGTTCGTTCTCG
AGCAAGAGGAATACAAAAAAGAGGGCATCGAATGGACTTT
CATCGATTTCGGTATGGATCTTCAGGCTTGTATAGATCTC
ATCGAGAAACCGATGGGGATTATGAGTATTCTGGAGGAGG
AATGTATGTTCCCGAAAGCCACTGACATGACATTTAAGGC
CAAGCTGTTCGATAATCACTTGGGGAAGTCCGCCAATTTC
CAGAAACCTAGGAACATAAAAGGTAAGCCGGAGGCGCACT
TCTCTCTTATCCATTACGCGGGAATCGTCGATTATAACAT
TATTGGCTGGCTTCAAAAGAACAAGGATCCGCTTAACGAA
ACAGTGGTCGGCCTTTATCAGAAAAGCTCACTGAAACTTC
TTAGTACGCTCTTTGCTAATTACGCTGGTGCTGACGCTCC
AATCGAGAAAGGCAAGGGAAAAGCTAAGAAAGGCAGTAGC
TTTCAAACAGTCTCTGCCCTGCACAGAGAGAATCTCAACA
AGCTCATGACCAATCTGCGGAGCACACATCCACATTTTGT
CAGGTGCATAATCCCTAATGAGACGAAAAGTCCAGGCGTA
ATGGACAATCCGCTGGTTATGCACCAGCTCAGATGTAATG
GAGTATTGGAAGGTATACGCATATGTAGAAAAGGCTTCCC
AAACAGGATACTGTATGGTGACTTCCGACAACGCTATCGA
ATACTGAACCCCGCAGCCATTCCCGAGGGACAATTCATAG
ACAGCCGCAAGGGAGCGGAAAAGCTTCTTTCCTCCCTCGA
CATAGACCATAACCAGTACAAATTTGGGCACACCAAAGTA
TTTTTCAAAGCGGGCCTTCTTGGACTGTTGGAAGAGATGC
GCGATGAAAGGCTGTCAAGGATAATAACCAGGATTCAAGC
GCAATCTCGGGGAGTGCTCGCGAGGATGGAGTACAAGAAA
TTGTTGGAACGGAGAGATTCCTTGCTTGTGATTCAGTGGA
ATATACGAGCTTTCATGGGAGTAAAAAACTGGCCTTGGAT
GAAACTCTACTTTAAGATAAAGCCACTGCTGAAGTCCGCA
GAGAGGGAGAAGGAGATGGCATCAATGAAGGAGGAATTTA
CGCGCTTGAAAGAGGCTCTCGAGAAGTCCGAAGCTAGACG
GAAGGAATTGGAAGAGAAGATGGTATCTCTTTTGCAGGAG
AAGAATGATCTTCAGTTGCAAGTACAAGCGGAACAAGACA
ATCTCGCCGACGCAGAGGAAAGGTGTGATCAGTTGATCAA
AAATAAAATCCAACTGGAAGCCAAGGTGAAAGAAATGAAT
GAGCGCTTGGAGGACGAAGAAGAGATGAATGCAGAGTTGA
CTGCGAAGAAGCGGAAGCTCGAAGACGAATGCCTGTCCTA
CGAGACCGAAATCCTGACCGTGGAGTATGGGCTGCTGCCC
ATCGGCAAGATTGTGGAGAAGCGGATTGAATGCACCGTGT
ATAGCGTGGACAACAACGGCAACATCTACACCCAGCCCGT
GGCTCAGTGGCACGACAGGGGCGAGCAGGAGGTGTTTGAG
TATTGTCTGGAGGACGGCAGCCTGATTAGAGCCACCAAAG
ACCACAAGTTCATGACCGTGGACGGGCAGATGCTGCCCAT
TGACGAGATTTTTGAGCGGGAACTTGACCTGATGCGGGTG
GACAACCTGCCCAACTGAACGCGTGCGGCCGCTTCCCTTT
AGTGAGGGTTAATGCTTCGAGCAGACATGATAAGATACAT
TGATGAGTTTGGACAAACCACAACTAGAATGCAGTGAAAA
AAATGCTTTATTTGTGAAATTTGTGATGCTATTGCTTTAT
TTGTAACCATTATAAGCTGCAATAAACAAGTTAACAACAA
CAATTGCATTCATTTTATGTTTCAGGTTCAGGGGGAGATG
TGGGAGGTTTTTTAAAGCAAGTAAAACCTCTACAAATGTG
GTAAAATCCGATAAGGGACTAGAGCATGGCTACGTAGATA
AGTAGCATGGCGGGTTAATCATTAACTACAAGGAACCCCT
AGTGATGGAGTTGGCCACTCCCTCTCTGCGCGCTCGCTCG
CTCACTGAGGCCGGGCGACCAAAGGTCGCCCGACGCCCGG
GCTTTGCCCGGGCGGCCTCAGTGAGCGAGCGAGCGCGCCA
GCTGGCGTAATAGCGAAGAGGCCCGCACCGATCGCCCTTC
CCAACAGTTGCGCAGCCTGAATGGCGAATGGAATTCCAGA
CGATTGAGCGTCAAAATGTAGGTATTTCCATGAGCGTTTT
TCCGTTGCAATGGCTGGCGGTAATATTGTTCTGGATATTA
CCAGCAAGGCCGATAGTTTGAGTTCTTCTACTCAGGCAAG
TGATGTTATTACTAATCAAAGAAGTATTGCGACAACGGTT
AATTTGCGTGATGGACAGACTCTTTTACTCGGTGGCCTCA
CTGATTATAAAAACACTTCTCAGGATTCTGGCGTACCGTT
CCTGTCTAAAATCCCTTTAATCGGCCTCCTGTTTAGCTCC
CGCTCTGATTCTAACGAGGAAAGCACGTTATACGTGCTCG
TCAAAGCAACCATAGTACGCGCCCTGTAGCGGCGCATTAA
GCGCGGCGGGTGTGGTGGTTACGCGCAGCGTGACCGCTAC
ACTTGCCAGCGCCCTAGCGCCCGCTCCTTTCGCTTTCTTC
CCTTCCTTTCTCGCCACGTTCGCCGGCTTTCCCCGTCAAG
CTCTAAATCGGGGGCTCCCTTTAGGGTTCCGATTTAGTGC
TTTACGGCACCTCGACCCCAAAAAACTTGATTAGGGTGAT
GGTTCACGTAGTGGGCCATCGCCCTGATAGACGGTTTTTC
GCCCTTTGACGTTGGAGTCCACGTTCTTTAATAGTGGACT
CTTGTTCCAAACTGGAACAACACTCAACCCTATCTCGGTC
TATTCTTTTGATTTATAAGGGATTTTGCCGATTTCGGCCT
ATTGGTTAAAAAATGAGCTGATTTAACAAAAATTTAACGC
GAATTTTAACAAAATATTAACGTCTACAATTTAAATATTT
GCTTATACAATCTTCCTGTTTTTGGGGCTTTTCTGATTAT
CAACCGGGGTACATATGATTGACATGCTAGTTTTACGATT
ACCGTTCATCGATTCTCTTGTTTGCTCCAGACTCTCAGGC
AATGACCTGATAGCCTTTGTAGAGACCTCTCAAAAATAGC
TACCCTCTCCGGCATGAATTTATCAGCTAGAACGGTTGAA
TATCATATTGATGGTGATTTGACTGTCTCCGGCCTTTCTC
ACCCGTTTGAATCTTTACCTACACATTACTCAGGCATTGC
ATTTAAAATATATGAGGGTTCTAAAAATTTTTATCCTTGC
GTTGAAATAAAGGCTTCTCCCGCAAAAGTATTACAGGGTC
ATAATGTTTTTGGTACAACCGATTTAGCTTTATGCTCTGA
GGCTTTATTGCTTAATTTTGCTAATTCTTTGCCTTGCCTG
TATGATTTATTGGATGTTGGAATCGCCTGATGCGGTATTT
TCTCCTTACGCATCTGTGCGGTATTTCACACCGCATATGG
TGCACTCTCAGTACAATCTGCTCTGATGCCGCATAGTTAA
GCCAGCCCCGACACCCGCCAACACCCGCTGACGCGCCCTG
ACGGGCTTGTCTGCTCCCGGCATCCGCTTACAGACAAGCT
GTGACCGTCTCCGGGAGCTGCATGTGTCAGAGGTTTTCAC
CGTCATCACCGAAACGCGCGAGACGAAAGGGCCTCGTGAT
ACGCCTATTTTTATAGGTTAATGTCATGATAATAATGGTT
TCTTAGACGTCAGGTGGCACTTTTCGGGGAAATGTGCGCG
GAACCCCTATTTGTTTATTTTTCTAAATACATTCAAATAT
GTATCCGCTCATGAGACAATAACCCTGATAAATGCTTCAA
TAATATTGAAAAAGGAAGAGTATGAGTATTCAACATTTCC
GTGTCGCCCTTATTCCCTTTTTTGCGGCATTTTGCCTTCC
TGTTTTTGCTCACCCAGAAACGCTGGTGAAAGTAAAAGAT
GCTGAAGATCAGTTGGGTGCACGAGTGGGTTACATCGAAC
TGGATCTCAACAGCGGTAAGATCCTTGAGAGTTTTCGCCC
CGAAGAACGTTTTCCAATGATGAGCACTTTTAAAGTTCTG
CTATGTGGCGCGGTATTATCCCGTATTGACGCCGGGCAAG
AGCAACTCGGTCGCCGCATACACTATTCTCAGAATGACTT
GGTTGAGTACTCACCAGTCACAGAAAAGCATCTTACGGAT
GGCATGACAGTAAGAGAATTATGCAGTGCTGCCATAACCA
TGAGTGATAACACTGCGGCCAACTTACTTCTGACAACGAT
CGGAGGACCGAAGGAGCTAACCGCTTTTTTGCACAACATG
GGGGATCATGTAACTCGCCTTGATCGTTGGGAACCGGAGC
TGAATGAAGCCATACCAAACGACGAGCGTGACACCACGAT
GCCTGTAGCAATGGCAACAACGTTGCGCAAACTATTAACT
GGCGAACTACTTACTCTAGCTTCCCGGCAACAATTAATAG
ACTGGATGGAGGCGGATAAAGTTGCAGGACCACTTCTGCG
CTCGGCCCTTCCGGCTGGCTGGTTTATTGCTGATAAATCT
GGAGCCGGTGAGCGTGGGTCTCGCGGTATCATTGCAGCAC
TGGGGCCAGATGGTAAGCCCTCCCGTATCGTAGTTATCTA
CACGACGGGGAGTCAGGCAACTATGGATGAACGAAATAGA
CAGATCGCTGAGATAGGTGCCTCACTGATTAAGCATTGGT
AACTGTCAGACCAAGTTTACTCATATATACTTTAGATTGA
TTTAAAACTTCATTTTTAATTTAAAAGGATCTAGGTGAAG
ATCCTTTTTGATAATCTCATGACCAAAATCCCTTAACGTG
AGTTTTCGTTCCACTGAGCGTCAGACCCCGTAGAAAAGAT
CAAAGGATCTTCTTGAGATCCTTTTTTTCTGCGCGTAATC
TGCTGCTTGCAAACAAAAAAACCACCGCTACCAGCGGTGG
TTTGTTTGCCGGATCAAGAGCTACCAACTCTTTTTCCGAA
GGTAACTGGCTTCAGCAGAGCGCAGATACCAAATACTGTC
CTTCTAGTGTAGCCGTAGTTAGGCCACCACTTCAAGAACT
CTGTAGCACCGCCTACATACCTCGCTCTGCTAATCCTGTT
ACCAGTGGCTGCTGCCAGTGGCGATAAGTCGTGTCTTACC
GGGTTGGACTCAAGACGATAGTTACCGGATAAGGCGCAGC
GGTCGGGCTGAACGGGGGGTTCGTGCACACAGCCCAGCTT
GGAGCGAACGACCTACACCGAACTGAGATACCTACAGCGT
GAGCTATGAGAAAGCGCCACGCTTCCCGAAGGGAGAAAGG
CGGACAGGTATCCGGTAAGCGGCAGGGTCGGAACAGGAGA
GCGCACGAGGGAGCTTCCAGGGGGAAACGCCTGGTATCTT
TATAGTCCTGTCGGGTTTCGCCACCTCTGACTTGAGCGTC
GATTTTTGTGATGCTCGTCAGGGGGGCGGAGCCTATGGAA
AAACGCCAGCAACGCGGCCTTTTTACGGTTCCTGGCCTTT
TGCTGGCCTTTTGCTCACATGTTCTTTCCTGCGTTATCCC
CTGATTCTGTGGATAACCGTATTACCGCCTTTGAGTGAGC
TGATACCGCTCGCCGCAGCCGAACGACCGAGCGCAGCGAG
TCAGTGAGCGAGGAAGCGGAAGAGCGCCCAATACGCAAAC
CGCCTCTCCCCGCGCGTTGGCCGATTCATTAATGCAGCTG
CGCGCTCGCTCGCTCACTGAGGCCGCCCGGGCAAAGCCCG
GGCGTCGGGCGACCTTTGGTCGCCCGGCCTCAGTGAGCGA
GCGAGCGCGCAGAGAGGGAGTGGCCAACTCCATCACTAGG
GGTTCCTTGTAGTTAATGATTAACCCGCCATGCTACTTAT
CTACGTAGCCATGCTCTAGATGTCATGGAGAAGACCCACC
TTGCAGATGTCCTCACTGGGGCTGGCAGAGCCGGCAACCT
GCCTAAGGCTGCTCAGTCCATTAGGAGCCAGTAGCCTGGA
AGATGTCTTTACCCCCAGCATCAGTTCAAGTGGAGCAGCA
CATAACTCTTGCCCTCTGCCTTCCAAGATTCTGGTGCTGA
GACTTATGGAGTGTCTTGGAGGTTGCCTTCTGCCCCCCAA
CCCTGCTCCCAGCTGGCCCTCCCAGGCCTGGGTTGCTGGC
CTCTGCTTTATCAGGATTCTCAAGAGGGACAGCTGGTTTA
TGTTGCATGACTGTTCCCTGCATATCTGCTCTGGTTTTAA
ATAGCTTATCTGAGCAGCTGGAGGACCACATGGGCTTATA
TGGCGTGGGGTACATGTTCCTGTAGCCTTGTCCCTGGCAC
CTGCCAAAATAGCAGCCAACACCCCCCACCCCCACCGCCA
TCCCCCTGCCCCACCCGTCCCCTGTCGCACATTCCTCCCT
CCGCAGGGCTGGCTCACCAGGCCCCAGCCCACATGCCTGC
TTAAAGCCCTCTCCATCCTCTGCCTCACCCAGTCCCCGCT
GAGACTGAGCAGACGCCTCCAGAGCTCGGATCCTGAGAAC
TTCAGGGTGAGTCTATGGGAC

SEQ ID NO: 9 below is an AAV vector encoding a second portion of MYH7 and a C-intein sequence:

GGCACTGGGCAGGTAAGTATCAAGGTTACAAGACAGGTTT
AAGGAGACCAATAGAAACTGGGCTTGTCGAGACAGAGAAG
ACTCTTGCGTTTCTGATAGGCACCTATTGGTCTTACTGAC
ATCCACTTTGCCTTTCTCTCCACAGGTGTCCACTCCCAGT
TCAATTACAGCTCTTAAGGCTAGAGTACTTAATACGACTC
ACTATAGGCTAGCGGTACCGGTCGCCACCATGATGATAAA
GATTGCCACCAGAAAGTATCTGGGCAAGCAGAACGTGTAT
GACATCGGCGTGGAGAGAGACCACAACTTCGCCCTGAAGA
ACGGCTTCATCGCCAGCAACTGCAGCGAGCTCAAGCGCGA
CATTGACGACCTGGAATTGACCCTTGCCAAAGTGGAAAAG
GAGAAACATGCAACCGAGAATAAGGTAAAGAATCTGACAG
AAGAGATGGCGGGACTTGACGAGATTATCGCGAAGCTTAC
AAAGGAGAAGAAGGCCCTTCAGGAAGCGCACCAACAGGCC
CTTGACGATCTTCAGGCCGAGGAAGACAAAGTGAACACAT
TGACTAAAGCTAAAGTGAAGCTTGAGCAACAGGTCGATGA
CCTGGAAGGCTCACTTGAGCAAGAGAAGAAAGTGCGCATG
GATCTCGAGCGCGCTAAACGCAAGCTCGAGGGAGATTTGA
AGCTGACTCAAGAGTCAATTATGGACCTTGAAAACGACAA
GCAACAGTTGGATGAGAGACTTAAGAAGAAGGACTTCGAA
CTCAACGCACTTAATGCCAGGATCGAAGATGAACAAGCTC
TCGGGTCTCAGCTCCAAAAGAAGCTTAAGGAACTCCAGGC
ACGCATAGAAGAACTCGAAGAAGAGTTGGAAGCCGAGAGA
ACGGCGAGAGCTAAAGTGGAAAAACTCCGAAGCGATTTGT
CCAGAGAGCTGGAAGAGATTTCCGAGCGGCTTGAGGAGGC
GGGAGGCGCGACGAGCGTGCAGATCGAAATGAATAAGAAG
AGAGAAGCTGAATTTCAAAAAATGCGCCGAGACCTTGAAG
AAGCAACACTCCAGCACGAAGCCACAGCAGCTGCGCTTCG
GAAGAAGCATGCTGATTCTGTCGCAGAGCTGGGGGAGCAG
ATCGACAATCTCCAGCGAGTCAAACAAAAACTGGAAAAGG
AGAAATCTGAGTTTAAATTGGAGCTTGACGACGTCACGTC
TAACATGGAGCAGATAATAAAGGCAAAAGCAAACCTTGAG
AAAATGTGCCGCACTCTTGAGGACCAGATGAACGAGCATC
GCAGTAAGGCGGAAGAGACTCAGAGATCTGTTAACGACTT
GACCAGTCAACGAGCTAAACTTCAGACAGAAAATGGAGAA
CTCTCCCGACAACTGGACGAAAAGGAGGCCCTGATCAGCC
AGTTGACTAGGGGTAAACTCACCTACACACAGCAGCTTGA
GGACTTGAAGCGCCAACTCGAAGAGGAGGTGAAAGCTAAA
AACGCACTGGCACATGCGCTGCAATCCGCAAGACATGACT
GCGACCTCCTGCGGGAACAGTACGAAGAAGAAACTGAGGC
GAAAGCTGAGCTCCAACGGGTACTTTCTAAGGCAAATTCT
GAAGTTGCACAGTGGCGAACTAAGTACGAAACCGATGCGA
TCCAACGCACGGAGGAGTTGGAGGAAGCTAAAAAGAAGCT
CGCACAGCGACTGCAGGAAGCCGAAGAAGCCGTTGAGGCA
GTTAATGCAAAATGCAGTAGTCTCGAGAAGACTAAACATA
GACTCCAGAACGAAATAGAGGATCTGATGGTGGATGTTGA
ACGCTCCAATGCTGCTGCGGCAGCCCTCGACAAAAAACAG
CGGAATTTCGATAAGATTCTTGCGGAATGGAAGCAGAAAT
ACGAAGAGTCTCAGAGTGAACTGGAGAGCTCCCAAAAGGA
GGCCCGAAGTCTGTCTACCGAATTGTTTAAACTCAAGAAC
GCGTACGAAGAATCTCTGGAACACTTGGAAACCTTTAAAA
GAGAGAACAAAAACCTGCAAGAGGAAATAAGTGACCTTAC
CGAGCAACTGGGGAGCTCCGGGAAAACCATCCACGAATTG
GAAAAAGTCAGGAAACAGTTGGAGGCAGAGAAAATGGAGC
TCCAATCCGCCCTGGAGGAAGCGGAAGCATCTCTGGAACA
CGAGGAGGGCAAGATTCTGAGGGCCCAACTGGAATTTAAC
CAGATCAAGGCAGAGATAGAACGAAAACTCGCGGAGAAGG
ACGAAGAGATGGAGCAGGCGAAACGGAATCATCTGCGGGT
TGTAGACTCCCTGCAAACCTCCCTCGATGCCGAAACCAGA
AGCCGGAATGAGGCCCTTCGGGTGAAAAAAAAAATGGAGG
GCGACTTGAACGAAATGGAAATTCAACTTTCTCACGCCAA
CCGCATGGCGGCCGAGGCGCAGAAACAGGTAAAATCTCTC
CAGTCTCTCCTCAAAGATACACAAATCCAACTCGACGATG
CTGTAAGGGCAAACGATGATTTGAAAGAGAATATAGCAAT
CGTCGAGCGCCGCAATAATCTGTTGCAAGCAGAGCTTGAA
GAACTGCGCGCGGTCGTAGAACAGACCGAACGCAGTAGAA
AGTTGGCTGAGCAGGAACTTATTGAGACTTCCGAGCGCGT
TCAGCTTCTGCATTCCCAGAACACCTCTCTGATTAATCAG
AAGAAAAAGATGGACGCAGATCTGTCTCAGTTGCAGACGG
AAGTGGAAGAAGCTGTTCAAGAGTGCCGGAACGCGGAGGA
AAAAGCTAAAAAGGCGATAACTGATGCAGCGATGATGGCT
GAGGAACTGAAGAAGGAGCAAGACACCTCCGCCCATTTGG
AGCGAATGAAGAAGAATATGGAACAAACTATTAAGGATCT
CCAGCACCGGCTTGATGAGGCTGAACAGATCGCCTTGAAA
GGGGGGAAAAAGCAATTGCAGAAATTGGAAGCCCGAGTAA
GGGAACTGGAGAATGAGTTGGAAGCTGAGCAAAAGCGGAA
CGCTGAGTCTGTGAAGGGAATGAGAAAGAGTGAACGGAGG
ATAAAAGAACTCACATATCAAACGGAAGAGGACCGGAAAA
ACTTGCTCCGCCTCCAAGACCTCGTTGACAAGCTTCAATT
GAAAGTCAAGGCTTATAAAAGACAAGCTGAAGAAGCTGAA
GAACAAGCGAACACCAATTTGTCCAAATTTCGGAAAGTAC
AGCATGAGCTCGACGAGGCTGAGGAGCGGGCTGACATAGC
TGAGTCCCAGGTCAATAAACTGCGAGCGAAAAGCAGAGAC
ATTGGCACCAAAGGTTTGAATGAAGAGACCGGTGAGGGCA
GAGGAAGTCTTCTAACATGCGGTGACGTGGAGGAGAATCC
CGGCCCTTCCGGTATGAAAACCTTCAACATCTCTCAGCAG
GATCTGGAGCTGGTGGAGGTCGCCACTGAGAAGATCACCA
TGCTCTATGAGGACAACAAGCACCATGTCGGGGCGGCCAT
CAGGACCAAGACTGGGGAGATCATCTCTGCTGTCCACATT
GAAGCCTACATTGGCAGGGTCACTGTCTGTGCTGAAGCCA
TTGCCATTGGGTCTGCTGTGAGCAACGGGCAGAAGGACTT
TGACACCATTGTGGCTGTCAGGCACCCCTACTCTGATGAG
GTGGACAGATCCATCAGGGTGGTCAGCCCCTGTGGCATGT
GTAGAGAGCTGATCTCTGACTATGCTCCTGACTGCTTTGT
GCTCATTGAGATGAATGGCAAGCTGGTCAAAACCACCATT
GAGGAACTCATCCCCCTCAAGTACACCAGGAACTAATAAG
CGGCCGCTTCCCTTTAGTGAGGGTTAATGCTTCGAGCAGA
CATGATAAGATACATTGATGAGTTTGGACAAACCACAACT
AGAATGCAGTGAAAAAAATGCTTTATTTGTGAAATTTGTG
ATGCTATTGCTTTATTTGTAACCATTATAAGCTGCAATAA
ACAAGTTAACAACAACAATTGCATTCATTTTATGTTTCAG
GTTCAGGGGGAGATGTGGGAGGTTTTTTAAAGCAAGTAAA
ACCTCTACAAATGTGGTAAAATCCGATAAGGGACTAGAGC
ATGGCTACGTAGATAAGTAGCATGGCGGGTTAATCATTAA
CTACAAGGAACCCCTAGTGATGGAGTTGGCCACTCCCTCT
CTGCGCGCTCGCTCGCTCACTGAGGCCGGGCGACCAAAGG
TCGCCCGACGCCCGGGCTTTGCCCGGGCGGCCTCAGTGAG
CGAGCGAGCGCGCCAGCTGGCGTAATAGCGAAGAGGCCCG
CACCGATCGCCCTTCCCAACAGTTGCGCAGCCTGAATGGC
GAATGGAATTCCAGACGATTGAGCGTCAAAATGTAGGTAT
TTCCATGAGCGTTTTTCCGTTGCAATGGCTGGCGGTAATA
TTGTTCTGGATATTACCAGCAAGGCCGATAGTTTGAGTTC
TTCTACTCAGGCAAGTGATGTTATTACTAATCAAAGAAGT
ATTGCGACAACGGTTAATTTGCGTGATGGACAGACTCTTT
TACTCGGTGGCCTCACTGATTATAAAAACACTTCTCAGGA
TTCTGGCGTACCGTTCCTGTCTAAAATCCCTTTAATCGGC
CTCCTGTTTAGCTCCCGCTCTGATTCTAACGAGGAAAGCA
CGTTATACGTGCTCGTCAAAGCAACCATAGTACGCGCCCT
GTAGCGGCGCATTAAGCGCGGCGGGTGTGGTGGTTACGCG
CAGCGTGACCGCTACACTTGCCAGCGCCCTAGCGCCCGCT
CCTTTCGCTTTCTTCCCTTCCTTTCTCGCCACGTTCGCCG
GCTTTCCCCGTCAAGCTCTAAATCGGGGGCTCCCTTTAGG
GTTCCGATTTAGTGCTTTACGGCACCTCGACCCCAAAAAA
CTTGATTAGGGTGATGGTTCACGTAGTGGGCCATCGCCCT
GATAGACGGTTTTTCGCCCTTTGACGTTGGAGTCCACGTT
CTTTAATAGTGGACTCTTGTTCCAAACTGGAACAACACTC
AACCCTATCTCGGTCTATTCTTTTGATTTATAAGGGATTT
TGCCGATTTCGGCCTATTGGTTAAAAAATGAGCTGATTTA
ACAAAAATTTAACGCGAATTTTAACAAAATATTAACGTCT
ACAATTTAAATATTTGCTTATACAATCTTCCTGTTTTTGG
GGCTTTTCTGATTATCAACCGGGGTACATATGATTGACAT
GCTAGTTTTACGATTACCGTTCATCGATTCTCTTGTTTGC
TCCAGACTCTCAGGCAATGACCTGATAGCCTTTGTAGAGA
CCTCTCAAAAATAGCTACCCTCTCCGGCATGAATTTATCA
GCTAGAACGGTTGAATATCATATTGATGGTGATTTGACTG
TCTCCGGCCTTTCTCACCCGTTTGAATCTTTACCTACACA
TTACTCAGGCATTGCATTTAAAATATATGAGGGTTCTAAA
AATTTTTATCCTTGCGTTGAAATAAAGGCTTCTCCCGCAA
AAGTATTACAGGGTCATAATGTTTTTGGTACAACCGATTT
AGCTTTATGCTCTGAGGCTTTATTGCTTAATTTTGCTAAT
TCTTTGCCTTGCCTGTATGATTTATTGGATGTTGGAATCG
CCTGATGCGGTATTTTCTCCTTACGCATCTGTGCGGTATT
TCACACCGCATATGGTGCACTCTCAGTACAATCTGCTCTG
ATGCCGCATAGTTAAGCCAGCCCCGACACCCGCCAACACC
CGCTGACGCGCCCTGACGGGCTTGTCTGCTCCCGGCATCC
GCTTACAGACAAGCTGTGACCGTCTCCGGGAGCTGCATGT
GTCAGAGGTTTTCACCGTCATCACCGAAACGCGCGAGACG
AAAGGGCCTCGTGATACGCCTATTTTTATAGGTTAATGTC
ATGATAATAATGGTTTCTTAGACGTCAGGTGGCACTTTTC
GGGGAAATGTGCGCGGAACCCCTATTTGTTTATTTTTCTA
AATACATTCAAATATGTATCCGCTCATGAGACAATAACCC
TGATAAATGCTTCAATAATATTGAAAAAGGAAGAGTATGA
GTATTCAACATTTCCGTGTCGCCCTTATTCCCTTTTTTGC
GGCATTTTGCCTTCCTGTTTTTGCTCACCCAGAAACGCTG
GTGAAAGTAAAAGATGCTGAAGATCAGTTGGGTGCACGAG
TGGGTTACATCGAACTGGATCTCAACAGCGGTAAGATCCT
TGAGAGTTTTCGCCCCGAAGAACGTTTTCCAATGATGAGC
ACTTTTAAAGTTCTGCTATGTGGCGCGGTATTATCCCGTA
TTGACGCCGGGCAAGAGCAACTCGGTCGCCGCATACACTA
TTCTCAGAATGACTTGGTTGAGTACTCACCAGTCACAGAA
AAGCATCTTACGGATGGCATGACAGTAAGAGAATTATGCA
GTGCTGCCATAACCATGAGTGATAACACTGCGGCCAACTT
ACTTCTGACAACGATCGGAGGACCGAAGGAGCTAACCGCT
TTTTTGCACAACATGGGGGATCATGTAACTCGCCTTGATC
GTTGGGAACCGGAGCTGAATGAAGCCATACCAAACGACGA
GCGTGACACCACGATGCCTGTAGCAATGGCAACAACGTTG
CGCAAACTATTAACTGGCGAACTACTTACTCTAGCTTCCC
GGCAACAATTAATAGACTGGATGGAGGCGGATAAAGTTGC
AGGACCACTTCTGCGCTCGGCCCTTCCGGCTGGCTGGTTT
ATTGCTGATAAATCTGGAGCCGGTGAGCGTGGGTCTCGCG
GTATCATTGCAGCACTGGGGCCAGATGGTAAGCCCTCCCG
TATCGTAGTTATCTACACGACGGGGAGTCAGGCAACTATG
GATGAACGAAATAGACAGATCGCTGAGATAGGTGCCTCAC
TGATTAAGCATTGGTAACTGTCAGACCAAGTTTACTCATA
TATACTTTAGATTGATTTAAAACTTCATTTTTAATTTAAA
AGGATCTAGGTGAAGATCCTTTTTGATAATCTCATGACCA
AAATCCCTTAACGTGAGTTTTCGTTCCACTGAGCGTCAGA
CCCCGTAGAAAAGATCAAAGGATCTTCTTGAGATCCTTTT
TTTCTGCGCGTAATCTGCTGCTTGCAAACAAAAAAACCAC
CGCTACCAGCGGTGGTTTGTTTGCCGGATCAAGAGCTACC
AACTCTTTTTCCGAAGGTAACTGGCTTCAGCAGAGCGCAG
ATACCAAATACTGTCCTTCTAGTGTAGCCGTAGTTAGGCC
ACCACTTCAAGAACTCTGTAGCACCGCCTACATACCTCGC
TCTGCTAATCCTGTTACCAGTGGCTGCTGCCAGTGGCGAT
AAGTCGTGTCTTACCGGGTTGGACTCAAGACGATAGTTAC
CGGATAAGGCGCAGCGGTCGGGCTGAACGGGGGGTTCGTG
CACACAGCCCAGCTTGGAGCGAACGACCTACACCGAACTG
AGATACCTACAGCGTGAGCTATGAGAAAGCGCCACGCTTC
CCGAAGGGAGAAAGGCGGACAGGTATCCGGTAAGCGGCAG
GGTCGGAACAGGAGAGCGCACGAGGGAGCTTCCAGGGGGA
AACGCCTGGTATCTTTATAGTCCTGTCGGGTTTCGCCACC
TCTGACTTGAGCGTCGATTTTTGTGATGCTCGTCAGGGGG
GCGGAGCCTATGGAAAAACGCCAGCAACGCGGCCTTTTTA
CGGTTCCTGGCCTTTTGCTGGCCTTTTGCTCACATGTTCT
TTCCTGCGTTATCCCCTGATTCTGTGGATAACCGTATTAC
CGCCTTTGAGTGAGCTGATACCGCTCGCCGCAGCCGAACG
ACCGAGCGCAGCGAGTCAGTGAGCGAGGAAGCGGAAGAGC
GCCCAATACGCAAACCGCCTCTCCCCGCGCGTTGGCCGAT
TCATTAATGCAGCTGCGCGCTCGCTCGCTCACTGAGGCCG
CCCGGGCAAAGCCCGGGCGTCGGGCGACCTTTGGTCGCCC
GGCCTCAGTGAGCGAGCGAGCGCGCAGAGAGGGAGTGGCC
AACTCCATCACTAGGGGTTCCTTGTAGTTAATGATTAACC
CGCCATGCTACTTATCTACGTAGCCATGCTCTAGATGTCA
TGGAGAAGACCCACCTTGCAGATGTCCTCACTGGGGCTGG
CAGAGCCGGCAACCTGCCTAAGGCTGCTCAGTCCATTAGG
AGCCAGTAGCCTGGAAGATGTCTTTACCCCCAGCATCAGT
TCAAGTGGAGCAGCACATAACTCTTGCCCTCTGCCTTCCA
AGATTCTGGTGCTGAGACTTATGGAGTGTCTTGGAGGTTG
CCTTCTGCCCCCCAACCCTGCTCCCAGCTGGCCCTCCCAG
GCCTGGGTTGCTGGCCTCTGCTTTATCAGGATTCTCAAGA
GGGACAGCTGGTTTATGTTGCATGACTGTTCCCTGCATAT
CTGCTCTGGTTTTAAATAGCTTATCTGAGCAGCTGGAGGA
CCACATGGGCTTATATGGCGTGGGGTACATGTTCCTGTAG
CCTTGTCCCTGGCACCTGCCAAAATAGCAGCCAACACCCC
CCACCCCCACCGCCATCCCCCTGCCCCACCCGTCCCCTGT
CGCACATTCCTCCCTCCGCAGGGCTGGCTCACCAGGCCCC
AGCCCACATGCCTGCTTAAAGCCCTCTCCATCCTCTGCCT
CACCCAGTCCCCGCTGAGACTGAGCAGACGCCTCCAGAGC
TCGGATCCTGAGAACTTCAGGGTGAGTCTATGGGAC

SEQ ID NO: 10 below is an AAV vector encoding a first portion of MHY7 and including a recombinogenic exogenous sequence:

GGCACTGGGCAGGTAAGTATCAAGGTTACAAGACAGGTTT
AAGGAGACCAATAGAAACTGGGCTTGTCGAGACAGAGAAG
ACTCTTGCGTTTCTGATAGGCACCTATTGGTCTTACTGAC
ATCCACTTTGCCTTTCTCTCCACAGGTGTCCACTCCCAGT
TCAATTACAGCTCTTAAGGCTAGAGTACTTAATACGACTC
ACTATAGGCTAGCGGTACCGGTCGCCACCATGGACTACAA
AGACCATGACGGTGATTATAAAGATCATGACATCGATTAC
AAGGATGACGATGACAAGCTTGAGCTCGGATCCATGGGAG
ATTCTGAAATGGCCGTCTTCGGCGCTGCCGCACCATATCT
CCGCAAAAGTGAAAAGGAGCGCCTGGAGGCCCAAACCAGA
CCCTTCGATCTGAAAAAGGATGTGTTTGTCCCGGATGACA
AGCAAGAATTTGTAAAAGCAAAGATCGTCTCTCGGGAGGG
CGGTAAAGTGACTGCTGAAACAGAATACGGCAAAACCGTC
ACTGTCAAAGAAGATCAGGTAATGCAACAAAATCCTCCGA
AATTTGATAAAATCGAAGACATGGCCATGTTGACGTTTTT
GCATGAGCCAGCTGTTTTGTATAATCTGAAGGATCGGTAC
GGATCCTGGATGATTTACACATACTCAGGCTTGTTCTGCG
TGACCGTTAATCCCTATAAGTGGCTGCCTGTCTACACGCC
GGAGGTTGTTGCAGCATATAGGGGGAAAAAGAGGTCCGAA
GCCCCTCCCCACATCTTCTCCATTTCTGACAACGCCTACC
AGTATATGCTGACTGACAGGGAGAACCAAAGCATTCTTAT
TACCGGAGAGTCTGGGGCGGGCAAGACGGTAAATACAAAA
CGCGTGATACAGTACTTTGCAGTTATTGCTGCGATTGGCG
ACAGGAGTAAAAAGGATCAGAGCCCCGGCAAGGGTACTCT
CGAAGATCAAATTATCCAGGCTAATCCCGCCCTCGAAGCG
TTCGGGAATGCCAAAACGGTCCGCAATGATAACAGTAGCA
GATTTGGGAAGTTTATCCGCATCCATTTCGGAGCGACTGG
CAAACTCGCCTCAGCAGACATCGAGACATACCTGTTGGAA
AAGTCACGAGTGATATTTCAGCTCAAAGCCGAGCGGGACT
ATCATATCTTTTACCAGATACTTTCTAACAAAAAACCAGA
GCTTCTTGACATGCTTCTGATCACTAACAATCCGTATGAC
TATGCATTCATCAGTCAGGGAGAGACCACGGTTGCGTCAA
TAGACGATGCTGAGGAGCTGATGGCCACAGACAATGCCTT
CGATGTGCTCGGATTTACTAGCGAGGAGAAGAACAGCATG
TACAAGCTTACCGGTGCTATCATGCACTTCGGCAATATGA
AATTTAAACTCAAGCAGCGGGAAGAACAGGCGGAGCCGGA
CGGAACTGAAGAGGCTGATAAAAGTGCCTACCTCATGGGG
CTTAACTCTGCCGACTTGCTTAAAGGATTGTGTCATCCAC
GGGTAAAGGTAGGTAATGAATATGTAACTAAGGGTCAAAA
TGTGCAACAAGTCATATACGCGACTGGGGCTCTGGCAAAA
GCCGTTTACGAGAGAATGTTTAACTGGATGGTCACACGAA
TTAATGCCACTCTGGAGACAAAACAACCCCGGCAGTACTT
TATAGGTGTGCTGGATATCGCAGGTTTCGAAATTTTCGAT
TTCAACTCTTTCGAGCAACTTTGTATCAATTTCACCAACG
AGAAGCTCCAACAATTTTTTAACCATCATATGTTCGTTCT
CGAGCAAGAGGAATACAAAAAAGAGGGCATCGAATGGACT
TTCATCGATTTCGGTATGGATCTTCAGGCTTGTATAGATC
TCATCGAGAAACCGATGGGGATTATGAGTATTCTGGAGGA
GGAATGTATGTTCCCGAAAGCCACTGACATGACATTTAAG
GCCAAGCTGTTCGATAATCACTTGGGGAAGTCCGCCAATT
TCCAGAAACCTAGGAACATAAAAGGTAAGCCGGAGGCGCA
CTTCTCTCTTATCCATTACGCGGGAATCGTCGATTATAAC
ATTATTGGCTGGCTTCAAAAGAACAAGGATCCGCTTAACG
AAACAGTGGTCGGCCTTTATCAGAAAAGCTCACTGAAACT
TCTTAGTACGCTCTTTGCTAATTACGCTGGTGCTGACGCT
CCAATCGAGAAAGGCAAGGGAAAAGCTAAGAAAGGCAGTA
GCTTTCAAACAGTCTCTGCCCTGCACAGAGAGAATCTCAA
CAAGCTCATGACCAATCTGCGGAGCACACATCCACATTTT
GTCAGGTGCATAATCCCTAATGAGACGAAAAGTCCAGGCG
TAATGGACAATCCGCTGGTTATGCACCAGCTCAGATGTAA
TGGAGTATTGGAAGGTATACGCATATGTAGAAAAGGCTTC
CCAAACAGGATACTGTATGGTGACTTCCGACAACGGTATC
GCATCCTGAACCCAGCGGCCATCCCTGAGGGACAGTTCAT
TGATAGCAGGAAGGGGGCAGAGAAGCTGCTCAGCTCCCTG
GACATTGATCACAACCAGTACAAGTTTGGCCACACCAAGG
TGAGTAAAGGAGACTAATTAATTAAAGGAAGACATCTCTG
TGATCCTAGGTGGAGGCCGAAAGTACATGTTTCGCATGGG
AACCCCAGACCCTGAGTACCCAGATGACTACAGCCAAGGT
GGGACCAGGCTGGACGGGAAGAATCTGGTGCAGGAATGGC
TGGCGAAGCGCCAGGGTGCCCGGTATGTGTGGAACCGCAC
TGAGCTCATGCAGGCTTCCCTGGACCCGTCTGTGACCCAT
CTCATGGGTCTCTTTGAGCCTGGAGACATGAAATACGAGA
TCCACCGAGACTCCACACTGGACCCCTCCCTGAATTGCAT
TCATTTTATGTTTCAGGTTCAGGGGGAGATGTGGGAGGTT
TTTTAAAGCAAGTAAAACCTCTACAAATGTGGTAAAATCC
GATAAGGGACTAGAGCATGGCTACGTAGATAAGTAGCATG
GCGGGTTAATCATTAACTACAAGGAACCCCTAGTGATGGA
GTTGGCCACTCCCTCTCTGCGCGCTCGCTCGCTCACTGAG
GCCGGGCGACCAAAGGTCGCCCGACGCCCGGGCTTTGCCC
GGGCGGCCTCAGTGAGCGAGCGAGCGCGCCAGCTGGCGTA
ATAGCGAAGAGGCCCGCACCGATCGCCCTTCCCAACAGTT
GCGCAGCCTGAATGGCGAATGGAATTCCAGACGATTGAGC
GTCAAAATGTAGGTATTTCCATGAGCGTTTTTCCGTTGCA
ATGGCTGGCGGTAATATTGTTCTGGATATTACCAGCAAGG
CCGATAGTTTGAGTTCTTCTACTCAGGCAAGTGATGTTAT
TACTAATCAAAGAAGTATTGCGACAACGGTTAATTTGCGT
GATGGACAGACTCTTTTACTCGGTGGCCTCACTGATTATA
AAAACACTTCTCAGGATTCTGGCGTACCGTTCCTGTCTAA
AATCCCTTTAATCGGCCTCCTGTTTAGCTCCCGCTCTGAT
TCTAACGAGGAAAGCACGTTATACGTGCTCGTCAAAGCAA
CCATAGTACGCGCCCTGTAGCGGCGCATTAAGCGCGGCGG
GTGTGGTGGTTACGCGCAGCGTGACCGCTACACTTGCCAG
CGCCCTAGCGCCCGCTCCTTTCGCTTTCTTCCCTTCCTTT
CTCGCCACGTTCGCCGGCTTTCCCCGTCAAGCTCTAAATC
GGGGGCTCCCTTTAGGGTTCCGATTTAGTGCTTTACGGCA
CCTCGACCCCAAAAAACTTGATTAGGGTGATGGTTCACGT
AGTGGGCCATCGCCCTGATAGACGGTTTTTCGCCCTTTGA
CGTTGGAGTCCACGTTCTTTAATAGTGGACTCTTGTTCCA
AACTGGAACAACACTCAACCCTATCTCGGTCTATTCTTTT
GATTTATAAGGGATTTTGCCGATTTCGGCCTATTGGTTAA
AAAATGAGCTGATTTAACAAAAATTTAACGCGAATTTTAA
CAAAATATTAACGTCTACAATTTAAATATTTGCTTATACA
ATCTTCCTGTTTTTGGGGCTTTTCTGATTATCAACCGGGG
TACATATGATTGACATGCTAGTTTTACGATTACCGTTCAT
CGATTCTCTTGTTTGCTCCAGACTCTCAGGCAATGACCTG
ATAGCCTTTGTAGAGACCTCTCAAAAATAGCTACCCTCTC
CGGCATGAATTTATCAGCTAGAACGGTTGAATATCATATT
GATGGTGATTTGACTGTCTCCGGCCTTTCTCACCCGTTTG
AATCTTTACCTACACATTACTCAGGCATTGCATTTAAAAT
ATATGAGGGTTCTAAAAATTTTTATCCTTGCGTTGAAATA
AAGGCTTCTCCCGCAAAAGTATTACAGGGTCATAATGTTT
TTGGTACAACCGATTTAGCTTTATGCTCTGAGGCTTTATT
GCTTAATTTTGCTAATTCTTTGCCTTGCCTGTATGATTTA
TTGGATGTTGGAATCGCCTGATGCGGTATTTTCTCCTTAC
GCATCTGTGCGGTATTTCACACCGCATATGGTGCACTCTC
AGTACAATCTGCTCTGATGCCGCATAGTTAAGCCAGCCCC
GACACCCGCCAACACCCGCTGACGCGCCCTGACGGGCTTG
TCTGCTCCCGGCATCCGCTTACAGACAAGCTGTGACCGTC
TCCGGGAGCTGCATGTGTCAGAGGTTTTCACCGTCATCAC
CGAAACGCGCGAGACGAAAGGGCCTCGTGATACGCCTATT
TTTATAGGTTAATGTCATGATAATAATGGTTTCTTAGACG
TCAGGTGGCACTTTTCGGGGAAATGTGCGCGGAACCCCTA
TTTGTTTATTTTTCTAAATACATTCAAATATGTATCCGCT
CATGAGACAATAACCCTGATAAATGCTTCAATAATATTGA
AAAAGGAAGAGTATGAGTATTCAACATTTCCGTGTCGCCC
TTATTCCCTTTTTTGCGGCATTTTGCCTTCCTGTTTTTGC
TCACCCAGAAACGCTGGTGAAAGTAAAAGATGCTGAAGAT
CAGTTGGGTGCACGAGTGGGTTACATCGAACTGGATCTCA
ACAGCGGTAAGATCCTTGAGAGTTTTCGCCCCGAAGAACG
TTTTCCAATGATGAGCACTTTTAAAGTTCTGCTATGTGGC
GCGGTATTATCCCGTATTGACGCCGGGCAAGAGCAACTCG
GTCGCCGCATACACTATTCTCAGAATGACTTGGTTGAGTA
CTCACCAGTCACAGAAAAGCATCTTACGGATGGCATGACA
GTAAGAGAATTATGCAGTGCTGCCATAACCATGAGTGATA
ACACTGCGGCCAACTTACTTCTGACAACGATCGGAGGACC
GAAGGAGCTAACCGCTTTTTTGCACAACATGGGGGATCAT
GTAACTCGCCTTGATCGTTGGGAACCGGAGCTGAATGAAG
CCATACCAAACGACGAGCGTGACACCACGATGCCTGTAGC
AATGGCAACAACGTTGCGCAAACTATTAACTGGCGAACTA
CTTACTCTAGCTTCCCGGCAACAATTAATAGACTGGATGG
AGGCGGATAAAGTTGCAGGACCACTTCTGCGCTCGGCCCT
TCCGGCTGGCTGGTTTATTGCTGATAAATCTGGAGCCGGT
GAGCGTGGGTCTCGCGGTATCATTGCAGCACTGGGGCCAG
ATGGTAAGCCCTCCCGTATCGTAGTTATCTACACGACGGG
GAGTCAGGCAACTATGGATGAACGAAATAGACAGATCGCT
GAGATAGGTGCCTCACTGATTAAGCATTGGTAACTGTCAG
ACCAAGTTTACTCATATATACTTTAGATTGATTTAAAACT
TCATTTTTAATTTAAAAGGATCTAGGTGAAGATCCTTTTT
GATAATCTCATGACCAAAATCCCTTAACGTGAGTTTTCGT
TCCACTGAGCGTCAGACCCCGTAGAAAAGATCAAAGGATC
TTCTTGAGATCCTTTTTTTCTGCGCGTAATCTGCTGCTTG
CAAACAAAAAAACCACCGCTACCAGCGGTGGTTTGTTTGC
CGGATCAAGAGCTACCAACTCTTTTTCCGAAGGTAACTGG
CTTCAGCAGAGCGCAGATACCAAATACTGTCCTTCTAGTG
TAGCCGTAGTTAGGCCACCACTTCAAGAACTCTGTAGCAC
CGCCTACATACCTCGCTCTGCTAATCCTGTTACCAGTGGC
TGCTGCCAGTGGCGATAAGTCGTGTCTTACCGGGTTGGAC
TCAAGACGATAGTTACCGGATAAGGCGCAGCGGTCGGGCT
GAACGGGGGGTTCGTGCACACAGCCCAGCTTGGAGCGAAC
GACCTACACCGAACTGAGATACCTACAGCGTGAGCTATGA
GAAAGCGCCACGCTTCCCGAAGGGAGAAAGGCGGACAGGT
ATCCGGTAAGCGGCAGGGTCGGAACAGGAGAGCGCACGAG
GGAGCTTCCAGGGGGAAACGCCTGGTATCTTTATAGTCCT
GTCGGGTTTCGCCACCTCTGACTTGAGCGTCGATTTTTGT
GATGCTCGTCAGGGGGGCGGAGCCTATGGAAAAACGCCAG
CAACGCGGCCTTTTTACGGTTCCTGGCCTTTTGCTGGCCT
TTTGCTCACATGTTCTTTCCTGCGTTATCCCCTGATTCTG
TGGATAACCGTATTACCGCCTTTGAGTGAGCTGATACCGC
TCGCCGCAGCCGAACGACCGAGCGCAGCGAGTCAGTGAGC
GAGGAAGCGGAAGAGCGCCCAATACGCAAACCGCCTCTCC
CCGCGCGTTGGCCGATTCATTAATGCAGCTGCGCGCTCGC
TCGCTCACTGAGGCCGCCCGGGCAAAGCCCGGGCGTCGGG
CGACCTTTGGTCGCCCGGCCTCAGTGAGCGAGCGAGCGCG
CAGAGAGGGAGTGGCCAACTCCATCACTAGGGGTTCCTTG
TAGTTAATGATTAACCCGCCATGCTACTTATCTACGTAGC
CATGCTCTAGATGTCATGGAGAAGACCCACCTTGCAGATG
TCCTCACTGGGGCTGGCAGAGCCGGCAACCTGCCTAAGGC
TGCTCAGTCCATTAGGAGCCAGTAGCCTGGAAGATGTCTT
TACCCCCAGCATCAGTTCAAGTGGAGCAGCACATAACTCT
TGCCCTCTGCCTTCCAAGATTCTGGTGCTGAGACTTATGG
AGTGTCTTGGAGGTTGCCTTCTGCCCCCCAACCCTGCTCC
CAGCTGGCCCTCCCAGGCCTGGGTTGCTGGCCTCTGCTTT
ATCAGGATTCTCAAGAGGGACAGCTGGTTTATGTTGCATG
ACTGTTCCCTGCATATCTGCTCTGGTTTTAAATAGCTTAT
CTGAGCAGCTGGAGGACCACATGGGCTTATATGGCGTGGG
GTACATGTTCCTGTAGCCTTGTCCCTGGCACCTGCCAAAA
TAGCAGCCAACACCCCCCACCCCCACCGCCATCCCCCTGC
CCCACCCGTCCCCTGTCGCACATTCCTCCCTCCGCAGGGC
TGGCTCACCAGGCCCCAGCCCACATGCCTGCTTAAAGCCC
TCTCCATCCTCTGCCTCACCCAGTCCCCGCTGAGACTGAG
CAGACGCCTCCAGAGCTCGGATCCTGAGAACTTCAGGGTG
AGTCTATGGGAC

SEQ ID NO: 11 below is an AAV vector encoding a second portion of MHY7 and including a recombinogenic exogenous sequence:

GGCCGCTTCCCTTTAGTGAGGGTTAATGCTTCGAGCAGAC
ATGATAAGATACATTGATGAGTTTGGACAAACCACAACTA
GAATGCAGTGAAAAAAATGCTTTATTTGTGAAATTTGTGA
TGCTATTGCTTTATTTGTAACCATTATAAGCTGCAATAAA
CAAGTTAACAACAACAATTGCATTCATTTTATGTTTCAGG
TTCAGGGGGAGATGTGGGAGGTTTTTTAAAGCAAGTAAAA
CCTCTACAAATGTGGTAAAATCCGATAAGGGACTAGAGCA
TGGCTACGTAGATAAGTAGCATGGCGGGTTAATCATTAAC
TACAAGGAACCCCTAGTGATGGAGTTGGCCACTCCCTCTC
TGCGCGCTCGCTCGCTCACTGAGGCCGGGCGACCAAAGGT
CGCCCGACGCCCGGGCTTTGCCCGGGCGGCCTCAGTGAGC
GAGCGAGCGCGCCAGCTGGCGTAATAGCGAAGAGGCCCGC
ACCGATCGCCCTTCCCAACAGTTGCGCAGCCTGAATGGCG
AATGGAATTCCAGACGATTGAGCGTCAAAATGTAGGTATT
TCCATGAGCGTTTTTCCGTTGCAATGGCTGGCGGTAATAT
TGTTCTGGATATTACCAGCAAGGCCGATAGTTTGAGTTCT
TCTACTCAGGCAAGTGATGTTATTACTAATCAAAGAAGTA
TTGCGACAACGGTTAATTTGCGTGATGGACAGACTCTTTT
ACTCGGTGGCCTCACTGATTATAAAAACACTTCTCAGGAT
TCTGGCGTACCGTTCCTGTCTAAAATCCCTTTAATCGGCC
TCCTGTTTAGCTCCCGCTCTGATTCTAACGAGGAAAGCAC
GTTATACGTGCTCGTCAAAGCAACCATAGTACGCGCCCTG
TAGCGGCGCATTAAGCGCGGCGGGTGTGGTGGTTACGCGC
AGCGTGACCGCTACACTTGCCAGCGCCCTAGCGCCCGCTC
CTTTCGCTTTCTTCCCTTCCTTTCTCGCCACGTTCGCCGG
CTTTCCCCGTCAAGCTCTAAATCGGGGGCTCCCTTTAGGG
TTCCGATTTAGTGCTTTACGGCACCTCGACCCCAAAAAAC
TTGATTAGGGTGATGGTTCACGTAGTGGGCCATCGCCCTG
ATAGACGGTTTTTCGCCCTTTGACGTTGGAGTCCACGTTC
TTTAATAGTGGACTCTTGTTCCAAACTGGAACAACACTCA
ACCCTATCTCGGTCTATTCTTTTGATTTATAAGGGATTTT
GCCGATTTCGGCCTATTGGTTAAAAAATGAGCTGATTTAA
CAAAAATTTAACGCGAATTTTAACAAAATATTAACGTCTA
CAATTTAAATATTTGCTTATACAATCTTCCTGTTTTTGGG
GCTTTTCTGATTATCAACCGGGGTACATATGATTGACATG
CTAGTTTTACGATTACCGTTCATCGATTCTCTTGTTTGCT
CCAGACTCTCAGGCAATGACCTGATAGCCTTTGTAGAGAC
CTCTCAAAAATAGCTACCCTCTCCGGCATGAATTTATCAG
CTAGAACGGTTGAATATCATATTGATGGTGATTTGACTGT
CTCCGGCCTTTCTCACCCGTTTGAATCTTTACCTACACAT
TACTCAGGCATTGCATTTAAAATATATGAGGGTTCTAAAA
ATTTTTATCCTTGCGTTGAAATAAAGGCTTCTCCCGCAAA
AGTATTACAGGGTCATAATGTTTTTGGTACAACCGATTTA
GCTTTATGCTCTGAGGCTTTATTGCTTAATTTTGCTAATT
CTTTGCCTTGCCTGTATGATTTATTGGATGTTGGAATCGC
CTGATGCGGTATTTTCTCCTTACGCATCTGTGCGGTATTT
CACACCGCATATGGTGCACTCTCAGTACAATCTGCTCTGA
TGCCGCATAGTTAAGCCAGCCCCGACACCCGCCAACACCC
GCTGACGCGCCCTGACGGGCTTGTCTGCTCCCGGCATCCG
CTTACAGACAAGCTGTGACCGTCTCCGGGAGCTGCATGTG
TCAGAGGTTTTCACCGTCATCACCGAAACGCGCGAGACGA
AAGGGCCTCGTGATACGCCTATTTTTATAGGTTAATGTCA
TGATAATAATGGTTTCTTAGACGTCAGGTGGCACTTTTCG
GGGAAATGTGCGCGGAACCCCTATTTGTTTATTTTTCTAA
ATACATTCAAATATGTATCCGCTCATGAGACAATAACCCT
GATAAATGCTTCAATAATATTGAAAAAGGAAGAGTATGAG
TATTCAACATTTCCGTGTCGCCCTTATTCCCTTTTTTGCG
GCATTTTGCCTTCCTGTTTTTGCTCACCCAGAAACGCTGG
TGAAAGTAAAAGATGCTGAAGATCAGTTGGGTGCACGAGT
GGGTTACATCGAACTGGATCTCAACAGCGGTAAGATCCTT
GAGAGTTTTCGCCCCGAAGAACGTTTTCCAATGATGAGCA
CTTTTAAAGTTCTGCTATGTGGCGCGGTATTATCCCGTAT
TGACGCCGGGCAAGAGCAACTCGGTCGCCGCATACACTAT
TCTCAGAATGACTTGGTTGAGTACTCACCAGTCACAGAAA
AGCATCTTACGGATGGCATGACAGTAAGAGAATTATGCAG
TGCTGCCATAACCATGAGTGATAACACTGCGGCCAACTTA
CTTCTGACAACGATCGGAGGACCGAAGGAGCTAACCGCTT
TTTTGCACAACATGGGGGATCATGTAACTCGCCTTGATCG
TTGGGAACCGGAGCTGAATGAAGCCATACCAAACGACGAG
CGTGACACCACGATGCCTGTAGCAATGGCAACAACGTTGC
GCAAACTATTAACTGGCGAACTACTTACTCTAGCTTCCCG
GCAACAATTAATAGACTGGATGGAGGCGGATAAAGTTGCA
GGACCACTTCTGCGCTCGGCCCTTCCGGCTGGCTGGTTTA
TTGCTGATAAATCTGGAGCCGGTGAGCGTGGGTCTCGCGG
TATCATTGCAGCACTGGGGCCAGATGGTAAGCCCTCCCGT
ATCGTAGTTATCTACACGACGGGGAGTCAGGCAACTATGG
ATGAACGAAATAGACAGATCGCTGAGATAGGTGCCTCACT
GATTAAGCATTGGTAACTGTCAGACCAAGTTTACTCATAT
ATACTTTAGATTGATTTAAAACTTCATTTTTAATTTAAAA
GGATCTAGGTGAAGATCCTTTTTGATAATCTCATGACCAA
AATCCCTTAACGTGAGTTTTCGTTCCACTGAGCGTCAGAC
CCCGTAGAAAAGATCAAAGGATCTTCTTGAGATCCTTTTT
TTCTGCGCGTAATCTGCTGCTTGCAAACAAAAAAACCACC
GCTACCAGCGGTGGTTTGTTTGCCGGATCAAGAGCTACCA
ACTCTTTTTCCGAAGGTAACTGGCTTCAGCAGAGCGCAGA
TACCAAATACTGTCCTTCTAGTGTAGCCGTAGTTAGGCCA
CCACTTCAAGAACTCTGTAGCACCGCCTACATACCTCGCT
CTGCTAATCCTGTTACCAGTGGCTGCTGCCAGTGGCGATA
AGTCGTGTCTTACCGGGTTGGACTCAAGACGATAGTTACC
GGATAAGGCGCAGCGGTCGGGCTGAACGGGGGGTTCGTGC
ACACAGCCCAGCTTGGAGCGAACGACCTACACCGAACTGA
GATACCTACAGCGTGAGCTATGAGAAAGCGCCACGCTTCC
CGAAGGGAGAAAGGCGGACAGGTATCCGGTAAGCGGCAGG
GTCGGAACAGGAGAGCGCACGAGGGAGCTTCCAGGGGGAA
ACGCCTGGTATCTTTATAGTCCTGTCGGGTTTCGCCACCT
CTGACTTGAGCGTCGATTTTTGTGATGCTCGTCAGGGGGG
CGGAGCCTATGGAAAAACGCCAGCAACGCGGCCTTTTTAC
GGTTCCTGGCCTTTTGCTGGCCTTTTGCTCACATGTTCTT
TCCTGCGTTATCCCCTGATTCTGTGGATAACCGTATTACC
GCCTTTGAGTGAGCTGATACCGCTCGCCGCAGCCGAACGA
CCGAGCGCAGCGAGTCAGTGAGCGAGGAAGCGGAAGAGCG
CCCAATACGCAAACCGCCTCTCCCCGCGCGTTGGCCGATT
CATTAATGCAGCTGCGCGCTCGCTCGCTCACTGAGGCCGC
CCGGGCAAAGCCCGGGCGTCGGGCGACCTTTGGTCGCCCG
GCCTCAGTGAGCGAGCGAGCGCGCAGAGAGGGAGTGGCCA
ACTCCATCACTAGGGGTTCCTTGTAGTTAATGATTAACCC
GCCATGCTACTTATCTACGTAGCCATGCTCTAGAGTGATC
CTAGGTGGAGGCCGAAAGTACATGTTTCGCATGGGAACCC
CAGACCCTGAGTACCCAGATGACTACAGCCAAGGTGGGAC
CAGGCTGGACGGGAAGAATCTGGTGCAGGAATGGCTGGCG
AAGCGCCAGGGTGCCCGGTATGTGTGGAACCGCACTGAGC
TCATGCAGGCTTCCCTGGACCCGTCTGTGACCCATCTCAT
GGGTCTCTTTGAGCCTGGAGACATGAAATACGAGATCCAC
CGAGACTCCACACTGGACCCCTCCCTACCCCTCCCTAGTC
ATGGCCAACACACACCTTGCCTGCAGGTGTTCTTCAAGGC
CGGGCTGCTGGGGCTGCTGGAGGAAATGAGGGACGAGAGG
CTGAGCCGCATCATCACGCGTATCCAGGCCCAGTCCCGAG
GTGTGCTCGCCAGAATGGAGTACAAAAAGCTGCTGGAACG
TAGAGATTCCTTGCTTGTGATTCAGTGGAATATACGAGCT
TTCATGGGAGTAAAAAACTGGCCTTGGATGAAACTCTACT
TTAAGATAAAGCCACTGCTGAAGTCCGCAGAGAGGGAGAA
GGAGATGGCATCAATGAAGGAGGAATTTACGCGCTTGAAA
GAGGCTCTCGAGAAGTCCGAAGCTAGACGGAAGGAATTGG
AAGAGAAGATGGTATCTCTTTTGCAGGAGAAGAATGATCT
TCAGTTGCAAGTACAAGCGGAACAAGACAATCTCGCCGAC
GCAGAGGAAAGGTGTGATCAGTTGATCAAAAATAAAATCC
AACTGGAAGCCAAGGTGAAAGAAATGAATGAGCGCTTGGA
GGACGAAGAAGAGATGAATGCAGAGTTGACTGCGAAGAAG
CGGAAGCTCGAAGACGAATGCAGCGAGCTCAAGCGCGACA
TTGACGACCTGGAATTGACCCTTGCCAAAGTGGAAAAGGA
GAAACATGCAACCGAGAATAAGGTAAAGAATCTGACAGAA
GAGATGGCGGGACTTGACGAGATTATCGCGAAGCTTACAA
AGGAGAAGAAGGCCCTTCAGGAAGCGCACCAACAGGCCCT
TGACGATCTTCAGGCCGAGGAAGACAAAGTGAACACATTG
ACTAAAGCTAAAGTGAAGCTTGAGCAACAGGTCGATGACC
TGGAAGGCTCACTTGAGCAAGAGAAGAAAGTGCGCATGGA
TCTCGAGCGCGCTAAACGCAAGCTCGAGGGAGATTTGAAG
CTGACTCAAGAGTCAATTATGGACCTTGAAAACGACAAGC
AACAGTTGGATGAGAGACTTAAGAAGAAGGACTTCGAACT
CAACGCACTTAATGCCAGGATCGAAGATGAACAAGCTCTC
GGGTCTCAGCTCCAAAAGAAGCTTAAGGAACTCCAGGCAC
GCATAGAAGAACTCGAAGAAGAGTTGGAAGCCGAGAGAAC
GGCGAGAGCTAAAGTGGAAAAACTCCGAAGCGATTTGTCC
AGAGAGCTGGAAGAGATTTCCGAGCGGCTTGAGGAGGCGG
GAGGCGCGACGAGCGTGCAGATCGAAATGAATAAGAAGAG
AGAAGCTGAATTTCAAAAAATGCGCCGAGACCTTGAAGAA
GCAACACTCCAGCACGAAGCCACAGCAGCTGCGCTTCGGA
AGAAGCATGCTGATTCTGTCGCAGAGCTGGGGGAGCAGAT
CGACAATCTCCAGCGAGTCAAACAAAAACTGGAAAAGGAG
AAATCTGAGTTTAAATTGGAGCTTGACGACGTCACGTCTA
ACATGGAGCAGATAATAAAGGCAAAAGCAAACCTTGAGAA
AATGTGCCGCACTCTTGAGGACCAGATGAACGAGCATCGC
AGTAAGGCGGAAGAGACTCAGAGATCTGTTAACGACTTGA
CCAGTCAACGAGCTAAACTTCAGACAGAAAATGGAGAACT
CTCCCGACAACTGGACGAAAAGGAGGCCCTGATCAGCCAG
TTGACTAGGGGTAAACTCACCTACACACAGCAGCTTGAGG
ACTTGAAGCGCCAACTCGAAGAGGAGGTGAAAGCTAAAAA
CGCACTGGCACATGCGCTGCAATCCGCAAGACATGACTGC
GACCTCCTGCGGGAACAGTACGAAGAAGAAACTGAGGCGA
AAGCTGAGCTCCAACGGGTACTTTCTAAGGCAAATTCTGA
AGTTGCACAGTGGCGAACTAAGTACGAAACCGATGCGATC
CAACGCACGGAGGAGTTGGAGGAAGCTAAAAAGAAGCTCG
CACAGCGACTGCAGGAAGCCGAAGAAGCCGTTGAGGCAGT
TAATGCAAAATGCAGTAGTCTCGAGAAGACTAAACATAGA
CTCCAGAACGAAATAGAGGATCTGATGGTGGATGTTGAAC
GCTCCAATGCTGCTGCGGCAGCCCTCGACAAAAAACAGCG
GAATTTCGATAAGATTCTTGCGGAATGGAAGCAGAAATAC
GAAGAGTCTCAGAGTGAACTGGAGAGCTCCCAAAAGGAGG
CCCGAAGTCTGTCTACCGAATTGTTTAAACTCAAGAACGC
GTACGAAGAATCTCTGGAACACTTGGAAACCTTTAAAAGA
GAGAACAAAAACCTGCAAGAGGAAATAAGTGACCTTACCG
AGCAACTGGGGAGCTCCGGGAAAACCATCCACGAATTGGA
AAAAGTCAGGAAACAGTTGGAGGCAGAGAAAATGGAGCTC
CAATCCGCCCTGGAGGAAGCGGAAGCATCTCTGGAACACG
AGGAGGGCAAGATTCTGAGGGCCCAACTGGAATTTAACCA
GATCAAGGCAGAGATAGAACGAAAACTCGCGGAGAAGGAC
GAAGAGATGGAGCAGGCGAAACGGAATCATCTGCGGGTTG
TAGACTCCCTGCAAACCTCCCTCGATGCCGAAACCAGAAG
CCGGAATGAGGCCCTTCGGGTGAAAAAAAAAATGGAGGGC
GACTTGAACGAAATGGAAATTCAACTTTCTCACGCCAACC
GCATGGCGGCCGAGGCGCAGAAACAGGTAAAATCTCTCCA
GTCTCTCCTCAAAGATACACAAATCCAACTCGACGATGCT
GTAAGGGCAAACGATGATTTGAAAGAGAATATAGCAATCG
TCGAGCGCCGCAATAATCTGTTGCAAGCAGAGCTTGAAGA
ACTGCGCGCGGTCGTAGAACAGACCGAACGCAGTAGAAAG
TTGGCTGAGCAGGAACTTATTGAGACTTCCGAGCGCGTTC
AGCTTCTGCATTCCCAGAACACCTCTCTGATTAATCAGAA
GAAAAAGATGGACGCAGATCTGTCTCAGTTGCAGACGGAA
GTGGAAGAAGCTGTTCAAGAGTGCCGGAACGCGGAGGAAA
AAGCTAAAAAGGCGATAACTGATGCAGCGATGATGGCTGA
GGAACTGAAGAAGGAGCAAGACACCTCCGCCCATTTGGAG
CGAATGAAGAAGAATATGGAACAAACTATTAAGGATCTCC
AGCACCGGCTTGATGAGGCTGAACAGATCGCCTTGAAAGG
GGGGAAAAAGCAATTGCAGAAATTGGAAGCCCGAGTAAGG
GAACTGGAGAATGAGTTGGAAGCTGAGCAAAAGCGGAACG
CTGAGTCTGTGAAGGGAATGAGAAAGAGTGAACGGAGGAT
AAAAGAACTCACATATCAAACGGAAGAGGACCGGAAAAAC
TTGCTCCGCCTCCAAGACCTCGTTGACAAGCTTCAATTGA
AAGTCAAGGCTTATAAAAGACAAGCTGAAGAAGCTGAAGA
ACAAGCGAACACCAATTTGTCCAAATTTCGGAAAGTACAG
CATGAGCTCGACGAGGCTGAGGAGCGGGCTGACATAGCTG
AGTCCCAGGTCAATAAACTGCGAGCGAAAAGCAGAGACAT
TGGCACCAAAGGTTTGAATGAAGAGGAGGGCAGAGGAAGT
CTTCTAACATGCGGTGACGTGGAGGAGAATCCCGGCCCTA
TGAAAACCTTCAACATCTCTCAGCAGGATCTGGAGCTGGT
GGAGGTCGCCACTGAGAAGATCACCATGCTCTATGAGGAC
AACAAGCACCATGTCGGGGCGGCCATCAGGACCAAGACTG
GGGAGATCATCTCTGCTGTCCACATTGAAGCCTACATTGG
CAGGGTCACTGTCTGTGCTGAAGCCATTGCCATTGGGTCT
GCTGTGAGCAACGGGCAGAAGGACTTTGACACCATTGTGG
CTGTCAGGCACCCCTACTCTGATGAGGTGGACAGATCCAT
CAGGGTGGTCAGCCCCTGTGGCATGTGTAGAGAGCTGATC
TCTGACTATGCTCCTGACTGCTTTGTGCTCATTGAGATGA
ATGGCAAGCTGGTCAAAACCACCATTGAGGAACTCATCCC
CCTCAAGTACACCAGGAACTAATAAGC

1. A method of treating or preventing cardiomyopathy in a human subject, the method comprising:
delivering a gene therapy drug to cardiac tissue of the human subject, the gene therapy drug comprising:
a first vector comprising a first portion of a polynucleotide sequence encoding for a therapeutic protein; and
a second vector comprising a second portion of the polynucleotide sequence encoding for the therapeutic protein.

2. The method of claim 1, wherein the first portion and the second portion of the polynucleotide sequence collectively define the entire polynucleotide sequence from its 5′ end to its 3′ end, wherein the first portion comprises a first continuous sequence starting from the 5′ end and ending upstream from the 3′ end, and wherein the second portion comprises a second continuous sequence starting downstream from the 5′ end and ending at the 3′ end.

3. The method of claim 2, wherein the first continuous sequence comprises a first overlap portion, wherein the second continuous sequence comprises a second overlap portion, wherein the first overlap portion overlaps with the second overlap portion, and wherein the first overlap portion and the second overlap portion are single-stranded and non-complementary to each other.

4. The method of claim 1, wherein the therapeutic protein comprises a functional MYH7 protein or functional variant thereof, and wherein the polynucleotide sequence encodes for the functional MYH7 protein or functional variant thereof.

5. The method of claim 4, wherein the first portion of the polynucleotide sequence comprises less than about half of the polynucleotide sequence starting from the 5′ end, and wherein the second portion of the polynucleotide sequence comprises a remainder of the polynucleotide sequence.

6. The method of claim 4, wherein the first portion of the polynucleotide sequence comprises more than about half of the polynucleotide sequence starting from the 5′ end, and wherein the second portion of the polynucleotide sequence comprises a remainder of the polynucleotide sequence.

7. The method of claim 4, wherein the first portion and the second portion of the polynucleotide sequence collectively define the polynucleotide sequence, wherein the first portion comprises a first continuous sequence starting from the 5′ end and ending upstream from the 3′ end, wherein the second portion comprises a second continuous sequence starting downstream from the 5′ end and ending at 3′ end, and wherein both the first continuous sequence and the second continuous sequence are single-stranded and non-complementary to each other.

8. The method of claim 7, wherein the first continuous sequence comprises a first overlap portion, wherein the second continuous sequence comprises a second overlap portion, and wherein the first overlap portion overlaps with the second overlap portion.

9. The method of claim 8, wherein the first overlap portion and the second overlap portion are each greater than 10 bases and less than 4,800 bases.

10. The method of claim 8, wherein the first overlap portion and the second overlap portion encode for intron 20 of the polynucleotide sequence.

11. The method of claim 8, wherein the first continuous sequence comprises exons 1 to 27 of the polynucleotide sequence, wherein the second continuous sequence comprises exons 19 to 40 of the polynucleotide sequence, and wherein the first overlap portion and the second overlap portion each comprises exons 19 to 27 of the polynucleotide sequence.

12-16. (canceled)

17. A gene therapy drug for treating or preventing cardiomyopathy in a human subject, the gene therapy drug comprising:
a first vector comprising a first portion of a polynucleotide sequence encoding for a therapeutic protein; and
a second vector comprising a second portion of the polynucleotide sequence encoding for the therapeutic protein.

18. The gene therapy drug of claim 17, wherein the first portion and the second portion of the polynucleotide sequence collectively define the entire polynucleotide sequence from its 5′ end to its 3′ end, wherein the first portion comprises a first continuous sequence starting from the 5′ end and ending upstream from the 3′ end, and wherein the second portion comprises a second continuous sequence starting downstream from the 5′ end and ending at the 3′ end.

19. The gene therapy drug of claim 18, wherein the first continuous sequence comprises a first overlap portion, wherein the second continuous sequence comprises a second overlap portion, wherein the first overlap portion overlaps with the second overlap portion, and wherein the first overlap portion and the second overlap portion are single-stranded and non-complementary to each other.

20. The gene therapy drug of claim 17, wherein the therapeutic protein comprises a functional MYH7 protein or functional variant thereof, and wherein the polynucleotide sequence is a polynucleotide sequence encoding for the functional MYH7 protein or functional variant thereof.

21. The gene therapy drug of claim 20, wherein the first portion of the polynucleotide sequence comprises less than about half of the polynucleotide sequence starting from the 5′ end, and wherein the second portion of the polynucleotide sequence comprises a remainder of the polynucleotide sequence.

22. The gene therapy drug of claim 20, wherein the first portion of the polynucleotide sequence comprises more than about half of the polynucleotide sequence starting from the 5′ end, and wherein the second portion of the polynucleotide sequence comprises a remainder of the polynucleotide sequence.

23. The gene therapy drug of claim 20, wherein the first portion and the second portion of the polynucleotide sequence collectively define the polynucleotide sequence, wherein the first portion comprises a first continuous sequence starting from the 5′ end and ending upstream from the 3′ end, wherein the second portion comprises a second continuous sequence starting downstream from the 5′ end and ending at 3′ end, and wherein both the first continuous sequence and the second continuous sequence are single-stranded and non-complementary to each other.

24. The gene therapy drug of claim 23, wherein the first continuous sequence comprises a first overlap portion, wherein the second continuous sequence comprises a second overlap portion, and wherein the first overlap portion overlaps with the second overlap portion.

25-33. (canceled)

34. A method of treating or preventing hypertrophic cardiomyopathy in a human subject, the method comprising:
delivering a gene therapy drug to cardiac tissue of the human subject, the gene therapy drug comprising:
a first rAAV2/9 vector comprising a continuous first portion of less than all of a polynucleotide sequence encoding for a functional MYH7 protein or functional variant thereof starting from the 5′ end and ending upstream from the 3′ end; and
a second rAAV2/9 vector comprising a continuous second portion of less than all of the polynucleotide sequence starting downstream from the 5′ end and ending at the 3′ end.

35-37. (canceled)
